(19) United States
(12) Patent Application Publication
(10) Pub. No.: US 2025/0351856 A1
(43) Pub. Date: Nov. 20, 2025

(54) FUNGAL INGREDIENTS AND DERIVED PRODUCTS

(71) Applicant: MUSHLABS GMBH, Hamburg (DE)

(72) Inventors: Thibault GODARD, Hamburg (DE); Wassim W. AYASS, Hamburg (DE); Max VÖLKER, Hamburg (DE); Marian NASSAR, Hamburg (DE); Tugce PARREIDT, Hamburg (DE); Fabian KÖRNER, Hamburg (DE); Christina STEIN, Hamburg (DE); Daniel Alejandro DELGADO MONTUFAR, Hamburg (DE)

(21) Appl. No.: 19/175,950

(22) Filed: Apr. 10, 2025

Related U.S. Application Data
(63) Continuation of application No. PCT/EP2023/079208, filed on Oct. 19, 2023.

(30) Foreign Application Priority Data
Oct. 19, 2022 (EP) 22202629.6
Oct. 19, 2022 (EP) 22202630.4

(51) Int. Cl. A23L 31/00; A23J 3/22; C12N 1/14

(52) U.S. Cl. CPC A23L 31/00; A23J 3/227; C12N 1/14

(57) ABSTRACT

The present invention relates to mycelium ingredients derived from submerged fermentation of at least one fungal strain in three different mediums: a defined medium leading to ingredient A, a synthetic medium leading to ingredient B, or a complex natural medium comprising a sidestream extract originated from an agrifood sidestream leading to ingredient C. Such unique mycelium compositions are characterized chemically, biologically, physically, morphologically, nutritionally, and organoleptically. The three different fibrous mycelium mass of edible fungi obtained from at least one fungal strain are further used to produce characterized food products, including meat analogues, fish analogues, dairy analogues, beverages, or other food products. These three new mycelium ingredients can be used in the manufacturing of foods, foodstuffs, beverages, pharmaceutical, cosmetics, nutraceutical, biomaterials, and feed and industrial applications.

### RELATED APPLICATIONS

This application is a continuation of International Patent Application No. PCT/EP2023/079208, filed Oct. 19, 2023, which claims priority to European Patent Application Nos. 22202630.4 and 22202629.6, both filed Oct. 19, 2022, the entire disclosures of which are hereby incorporated herein by reference.

### FIELD OF INVENTION

The present invention relates to mycelium ingredients derived from submerged fermentation of at least one fungal strain in three different mediums: a defined medium leading to ingredient A, a synthetic medium leading to ingredient B, or a complex natural medium comprising a sidestream extract selected from an agrifood sidestream leading to ingredient C. Such unique mycelium compositions are characterized chemically, biologically, physically, morphologically, nutritionally, and organoleptically. The three different fibrous mycelium mass of edible fungi obtained from at least one fungal strain are further used to produce characterized food products, including meat analogues, fish analogues, dairy analogues, beverages, or other food products. These three new mycelium ingredients can be used in the manufacturing of foods, foodstuffs, beverages, pharmaceutical, cosmetics, nutraceutical, biomaterials, and feed and industrial applications.

### BACKGROUND OF THE INVENTION

In the last 10 years, several food scandals have drawn unprecedented attention to our current food production systems and their lack of clarity and robustness when it comes to food safety. In 2011, long international supply chains were thought to be the main driver for the enteroaggregative E. coli outbreak in sprouts that led to several deaths across Europe, as well as the recall of related products worldwide. Later, in 2013, significant amounts of horse meat were found in products advertised as beef across Europe with potential health implications related to contamination with phenylbutazone, a common analgesic for horses. In the same year, the Muslim and Jewish communities were impacted when pig meat was found in beef products. In 2017, eggs contaminated with Finopril, a common insecticide, were found in several European and Asian countries. In all these cases, determining the origin of contamination and then coordinating the removal of spoiled foods were hindered by the complexity of current global supply and distribution channels, thus putting consumers at risk over a prolonged period.

On a different level, the COVID-19 pandemic has also clearly highlighted that global food systems do not offer the required level of resilience with respect to food security. The first year of the pandemic led to an increase in global food prices by around 20% and the World Food Program (WFP) estimates that the number of people suffering from acute food insecurity increased from 135 to 272 million (worldbank.org/en/topic/agriculture/brief/food-security-and-covid-19 and csis.org/analysis/covid-19-and-global-food-security-one-year-later, both assessed on Oct. 17, 2022). By analogy, the food system is vulnerable against worldwide catastrophes.

At the same time, one third of all the food produced worldwide is either lost before it reaches the consumer or wasted afterwards. This represents around 1.3 bn tons of food, much of which could potentially be reclaimed with optimal production and distribution logistics. Rising consumer awareness has led to the emergence of new purchasing trends where local, natural, healthy, and sustainable products are favored over ultra-processed unbalanced foods. Modern consumers expect to be able to track the origin of what they buy and understand the ingredients on the package, as well as the impact of the product on their health and that of our planet.

Environmental awareness has increased in recent years as the unsustainability and, in some cases, cruelty in industrial production methods and practices for meat and fish have been brought to light by several published studies referenced here (doi.org/10.3390/foods9091227 and doi.org/10.3390/foods9091151). While plant-based alternatives can significantly cut down CO₂ emissions and improve animal welfare compared to traditional meat production, they do not fully address the challenge of local production, as these products are derived mostly from three monocrops (soy, pea and rice) that are only grown in a handful of countries and need to be exported worldwide. Moreover, their cultivation requires large land areas that are unfortunately often acquired through deforestation and their efficient production still relies heavily on chemical agents, such as pesticides and fertilizers, that contribute to soil and water pollution and have a lasting impact on biodiversity. In addition, only concentrates and isolates from the crops are used in the production of meat alternatives and significant amounts of waste are therefore generated in the process. Finally, these plant proteins have a strong bitter taste and no intrinsic texture. Therefore, their use in foods requires further processing steps and a long list of ingredients. Hence, plant-based alternatives may not address all consumer concerns with respect to traceability and sustainability of foods.

In parallel to the development of plant-based meat alternatives, some traditional foods, such as mushrooms, have also received increased attention for their potential as natural meat replacers in terms of nutrition, texture and/or taste. Mushrooms are particularly interesting because they have a natural umami taste, with certain variations between species that enable the production of products with a taste profile close to meat or other savory foods without adding a long list of ingredients. Moreover, their fruiting body or cap also has a texture that resembles meat and can be further improved with minimal processing for specific applications. In terms of nutrition, mushrooms contain up to 40% complete protein but also prebiotic fibers that are often lacking in western diets. Mushrooms also contain large amounts of key minerals, such as iron, zinc, calcium, potassium or magnesium, and vitamins from the B group; they can be considered as one of the rare foods to provide a complete and balanced nutritional profile.

The culturing of mushrooms is a lengthy process, although highly environmentally friendly. In nature, mushrooms are equipped with a wide range of unique enzymes that enable them to scavenge waste materials, such as fallen leaves or wood residues, present on the soil of forests. They can degrade complex plant compounds that are otherwise generally not accessible nutrients for other groups of organisms. This feature has made mushrooms ideal candidates to upcycle wastes from the agrifood industry that are often highly unstable and generally used as feed, burnt, or simply discarded despite their residual nutrient content (e.g., wheat straw or plant husks, etc.). To date, only around 144,000 fungal species have been described and it is estimated that there are over 10 million species on the planet, including a wide range of unknown edible mushroom species. Many of these undiscovered species are likely to offer new avenues of culinary experience and upcycling opportunities for waste materials. Despite their attractive attributes as food products, mushrooms are relatively slow-growers, and a production cycle generally takes at least 6 weeks. Moreover, the traditional production methods are very basic and use techniques that are difficult to scale, such as growth on forest trees and/or in bags containing lignocellulosic material. In recent years, more modern techniques involving the use of incubation chambers, in which temperature and humidity are tightly controlled, or hydroponics have enabled significant improvement in process standardization and production yields, but they require large investments and do not fully address the scalability problem.

The use of fermentation to produce mushroom mycelium in this context presents advantages in terms of sustainability, food safety and traceability. Fermenters are sterile vessels operating under controlled conditions; hence, the risk of spoilage is reduced to a minimum. They can be scaled vertically and therefore allow a smaller plant footprint as well as the potential to produce food locally using by-products from farms or food processors, removing the need for long supply or distribution chains. Furthermore, production of mushroom mycelium in fermenters is also more sustainable than producing traditional plant-based alternatives. Water consumption, land area requirement, energy consumption, CO₂ emissions are estimated to be lower than for traditional meat alternatives from soy.

Therefore, to circumvent the above-mentioned problems and limitations, three novel mushroom mycelium ingredients or fungal biomass ingredients were developed in a liquid medium, via a liquid-state or submerged fermentation, wherein the taste, composition (carbohydrates, fats, protein, nutrients, vitamins, fiber content, amino acids, etc.), texture and structure is controlled during the fermentation process depending on the fermentation conditions used (e.g., medium used, species, process configuration and conditions) leading to unique biological, physical and chemical properties for each mycelium ingredient. In addition, the biomass keeps typical umami flavor, which can also be controlled at the fermentation level, when cooked so that food products, such as the meat analogues and dairy analogues or other food products developed in this invention, made with the mycelium flesh or ingredients developed and disclosed in this invention, require minimal processing and a very short list of ingredients that can be easily communicated to the customer.

### SUMMARY OF THE INVENTION

The present invention solves the above-mentioned challenges by introducing three new raw materials or ingredients to meat, fish and dairy analogues production, or other food products, namely mycelia of edible mushrooms. These three new ingredients can also be used in the manufacturing of foods, foodstuffs, beverages, pharmaceutical, cosmetics, nutraceutical, biomaterials, and feed and industrial applications. Mycelium or filamentous fungi has been broadly applied in meat-replacement products because of their filamentous structure (GB2137226A) and as fat-mimicking substances in dairy drinks or yoghurts (WO2002090527A1).

CN103184246A discloses a preparation method of ergothioneine utilizing a liquid culture of wild Pleurotus sapidus, Pleurotus pulmonarius or Lepisa sordida to produce ergothioneine with a low yield of 51 mg/L with a cultivation time of 10 days. For example, CN110283856A discloses a method for producing ergothioneine by fermenting the fungal strain Pleurotus ostreatus of 3210 with a yield of 300 mg/L, however the process needs at least 25 days between having the mycelium grow on PD for 15 days followed by a fermentation time for 10 days. It was observed in the patent literature that co-fermenting more than one fungal strain can yield to a higher content of ergothioneine (CN112195215 or CN114214387). And lastly CN109939027A discloses a method for producing ergothioneine by fermenting Hericium erinaceus with glucose and peptone, with a yield of 331 mg/L, however, the production cost of the substrates is high, and the process takes a long time (around 25 days).

CN212786880 reports that Pleurotus pulmonarius fruiting body is low in fiber content supporting the review published in 2021 (Fungal Biotec 1(2):65-87 (2021)) summarizing the fiber content of the fruiting body of Pleurotus spp. that ranges from 2.97 wt. % to at most 31 wt. %, in particular the fruiting body of Pleurotus pulmonarius contains 4-9 wt. % fiber on dry basis. CN105054261 also discloses the finding that when Pleurotus pulmonarius is mixed with other strains, a degradation of crude fibers takes place, especially on oyster mushrooms (Pleurotus spp.), thus reducing the fiber content by 2.3-20.25 wt. %. Another study in 2020 (Int J Med Mushrooms. 2020; 22(7):651-657.doi: 10.1615/IntJMedMushrooms.2020035449) also reveals that mycelia of oyster mushrooms (Pleurotus spp.) contains 22 wt. % of insoluble fibers. Document US 2020/270559 discloses certain methods of production of edible filamentous fungal biomat formulations and shows nutritional data from two Fusarium filamentous fungi having a total fiber content up to 25 wt. % and a fat content between 7 wt. % and 12 wt. %.

On another point, the RNA level of the currently available mycelium ingredients are usually actively reduced via a process involving a final treating step comprising of a heating step at a certain temperature and/or at an adjusted pH for a certain time to actively reduce the RNA content to less than 4%, preferably less than 2% on dry matter basis, thus reducing unwanted related health risks and bitter tastes and meeting the regulatory requirements, as disclosed in the developed methods in these publications U.S. Pat. Nos. 4,041,189, 4,501,765, WO201802579.

Jeng-Leun Mau (2015) summarized the equivalence umami concentration (EUC) values of fruit bodies and showed that it varied widely and ranged from a highest value of 4465% (Volvariella volvacea fl at cap) to a lowest value of 0.12% (Auricularia polytricha). EUC values are grouped into four levels: >1000% (>10 g MSG/g dry matter), 100-1000% (1-10 g MSG/g), 10-100% (0.1-1 g MSG/g), and 10% (<0.1 g MSG/g), wherein MSG is the equivalent concentration of monosodium glutamate (International Journal of Medicinal Mushrooms, Vol. 7, pp. 119-125 (2005)). Among the EUC values of Pleurotus species, P. citrinopileatus was the highest (511% at the second level), and the others were in the descending order of P. eryngii small fruit body (97.9%), P. cystidiosus (85.2%), and P. ostreatus (48.0%). Based on the forms of fruit bodies in cultivation bottles or plastic bags (logs), the EUC values of P. eryngii were 68.7, 97.9 and 32.1% for large and small fruit bodies and base, respectively. In a separate study, P. pulmonarius fruiting bodies were grown on three forestry wastes (pine, poplar, and honeysuckle rattan), showing EUC values of P. pulmonarius fruiting bodies between 72.31% and 116.73% (Food Chemistry 397 (2022) 133714). It is to be understood that EUC is preferably expressed in g MSG/100 g dry matter. For example, an EUC concentration of 1000% (or 1000 wt. %) is equivalent to 1000 g MSG/100 g dry matter.

In 1958, Eddy et al. reported several unsuccessful trials based on patented inventions such as U.S. Pat. No. 2,693,664 to enhance the flavour of mycelium derived from submerged fermentation (J. Sci. Food Agric. 9 1958).

WO2022/107388 discloses a method providing an umami enhancing composition of mushrooms of the genus Flammulina via enzymatic treatment reaching to a result of a maximum reported equivalent umami concentration (EUC) value of 9.3 g/100 g.

CN114027089 discloses a method of improving flavor of edible mushrooms wherein the equivalent umami concentration values of the Flammulina velutipes obtained by adding edible fungus root fermentation liquid increased the EUC from 4.48 g/100 g to 10.8 g of MSG/100 g.

CN109156702 A discloses a soaking method of Hericium erinaceus to decrease the original EUC from 1131 g MSG/100 g to the following values by these following various treatments, namely steam treatment (959.82 g MSG/100 g), water bath treatment (755.39 g MSG/100 g) and ultrasonic treatment (189.84 g MSG/100 g).Similar studies were reported in Li-bin Sun et al (Trends in Food Science & Technology 96 (2020) 176-187), were mushroom fruiting bodies, not mycelium, were treated in various physical methods leading to different EUC concentrations of the fruiting bodies.

KR101535985 relates to a method including a step of initiating the Maillard reaction between the powder of one or more mushroom varieties selected from a group including the bearded tooth mushrooms, shiitake mushrooms (Lentinula edodes), oyster mushrooms (Pleurotus ostreatus), and enoki mushrooms (Flammulina velutipes) and other seasoning ingredients to reach a value of EUC of 176 mg MSG/100 g. It is noted that performed measurements concern fruiting bodies of the fungi at issue, and not their mycelium.

Herein provided are three novel mycelium ingredients, A, B, and C, which are unique chemically, biologically, physically, nutritionally, and organoleptically (i.e., characterized by an improved-strengthened, or reduced taste, as applicable).

In one embodiment of the invention, the provided mycelia are grown in three different mediums via submerged fermentation of at least one fungal strain in a: defined medium leading to ingredient A, synthetic medium leading to ingredient B, or a natural medium comprising a sidestream extract selected from an agrifood sidestream leading to ingredient C.

In a particular embodiment of this invention, the provided mycelia have a different carbon to nitrogen ratio, similar chitin content, but a different portfolio of sugar content (sugars, polysaccharides, oligosachharides).

In a further specific embodiment of this invention, the provided mycelia have a low inherent RNA level of at most 4 wt. %, preferably at most 2 wt. %, that avoids the need to have an extra treatment to actively reduce the RNA levels.

In a further specific embodiment of this invention, the provided mycelia have an ergothioneine content up to 800 mg/kg. Said ergothioneine content can be achieved in a shorter time compared to time reported the prior art.

In a further specific embodiment of this invention, the provided mycelia have specific/unique pore volumes, pore size distributions and/or a characterized texture.

In a further specific embodiment of this invention, in terms of their adjustable insoluble fiber content of at least 20 wt. %, at least 30 wt. % at least 40 wt. %, at least 50 wt. % or at least 60 wt. % offering an even higher health value of its high prebiotic insoluble fiber content critical for gut health.

In a further specific embodiment of this invention, in terms of their amino acid content, wherein the amount of the branched-chain amino acids (BCAA) is at least about 19 wt. %, at least about 20 wt. % of the total amount of amino acids present. (i.e., of the total protein).

In a further specific embodiment of this invention, in terms of their adjustable protein content ranging between 10 wt. % and 65 wt. %.

In a further specific embodiment of this invention the umami amino acids in the mycelium ingredients are at least about 20 wt. % of the total amount of amino acids present.

In a further specific embodiment, the mycelia coming from at least one fungal strain, are mixed with at least one protein rich ingredient, at least one lipid rich ingredient, and at least one compositional ingredient to produce meat or dairy analogues.

In a further specific embodiment, the mycelia coming from at least one fungal strain have a very high equivalence umami concentration (EUC) ranging up to at least 34%, more preferably at least 300%, even more preferably at least 2800% which is about 24 to 40-fold higher compared to fruiting bodies of mushrooms of the same species and higher than reported EUC values of mycelia as discussed in the invention.

Upon application of the mycelial ingredient of the present invention (any of A, B and C), flavour can also be significantly improved as the mycelia can bring a natural umami flavour typical to dairy, fish or meat analogues without the usage of additional flavours, especially in the case when mycelium originates from a fungus that forms fruiting bodies, e.g. from Pleurotaceae, such as fungus selected from Pleurotus pulmonarius, Pleurotus ostreatus, Pleurotus citrinopileatus, Pleurotus florida, and Pleurotus salmoneostramineus, and specifically if derived from Pleurotus pulmonarius; or from a fungus selected from Morchella esculenta, Morchella angusticeps, Morchella deliciosa, and Morchella rufobrunnea, preferably Morchella rufobrunnea. This is due to a significant amount of glutamate present in such species along with other amino acids (e.g., aspartate) and/or umami 5′-nucleotides, which are in large part responsible for conveying the umami sensation.

In a further embodiment, the present invention relates to a method for producing a fungal biomass by submerged fermentation of at least one fungal strain, wherein the at least one fungal strain is an edible fungus.

In again a further embodiment, the present invention relates to a method for producing a fungal biomass by submerged fermentation of at least one fungal strain, wherein the submerged fermentation is operated as a batch, a fed-batch or a continuous process.

In again a further embodiment, the present invention relates to a method for producing a fungal biomass by submerged fermentation of at least one fungal strain, wherein at least two fungal strains are co-fermented.

In a further embodiment, the present invention relates to a fungal biomass produced according to the method for producing a fungal biomass by submerged fermentation of at least one fungal strain of the present invention, wherein the fungal strain is selected from Pleurotaceae, in particular wherein the fungal strain is P. pulmonarius, P. ostreatus, P. citrinopileatus or P. salmoneostramineus.

In a particular embodiment, the present invention relates to a fungal biomass produced according to the method for producing a fungal biomass by submerged fermentation of at least one fungal strain of the present invention, wherein the fungal strain is selected from Morchellaceae, in particular wherein the fungal strain is M. esculenta, M. angusticeps or M. deliciosa.

In an alternative particular embodiment, the edible fibrous mycelium mass is obtained from at least one fungal strain that preferably can produce ergothioneine. Said fungal strain is preferably selected from Basidiomycota, Ascomycota, Hymenochaetaceae, Agaricomycetes, Sordariomycetes, Tremellomycetes, wherein the preferable species herein are from at least one fungal species selected from Cordyceps spp., Inonotus spp., Grifola spp., Pleurotus spp., Ganoderma spp., Lentinula spp., Tremella spp., Trametes spp., Lepista spp., Tricholoma spp., Aspergillus spp., and/or Panus spp.

The present invention is also concerned with methods of producing the above-mentioned edible meat, fish and dairy substitute products comprising the edible fibrous mycelium mass of the present invention. These methods are described in detail below.

The present invention is also concerned with the use of an edible fibrous mycelium mass for producing an edible meat substitute product, wherein the edible meat substitute product is selected from products substituting meatballs, sausages, tartar, minced meat, meat spreads, processed meat, Mett meat, foie gras, steak, beef jerky, burger patty, fillet, nugget, salami, whole-cuts, bacon, hot dogs, prosciutto, dried meat and extruded products.

The present invention is also concerned with the use of an edible fibrous mycelium mass for producing an edible dairy substitute product, wherein the edible dairy substitute product is selected from products substituting milk, yoghurt, fresh cheese, whey cheese, cream cheese, medium-hard cheese, hard-cheese, and soft-mould cheese.

The present invention is also concerned with the use of an edible fibrous mycelium mass for producing an edible fish analogues or seafood products, for example a crabcake, fishcake, tuna, salmon, or shrimp.

In a further embodiment, the present invention relates to a fungal-based food product prepared using the fungal biomass of the present invention.

In a further embodiment, the present invention is also concerned with the use of the supernatant produced during the fermentation to develop specific health drinks containing antioxidant and a specific aroma, taste and flavors developed during fermentation with edible mushrooms. Mushroom strains are known to produce pleasant volatiles and other compounds with apple or almond taste, for example. They also produce compounds known to regulate the blood sugar level.

The present invention is also concerned with the use of the supernatant to be further processed, e.g., to extract its particular components, e.g., proteins, in particular enzymes produced by microorganism(s) cultured in the medium, polysaccharides, peptides, antioxidants, etc.

### BRIEF DESCRIPTION OF FIGURES

FIG. 1 shows the overlay of pore size distribution curves (−dV/d log D) of mycelium ingredients A, B, and C which are calculated from normalized volume curves.

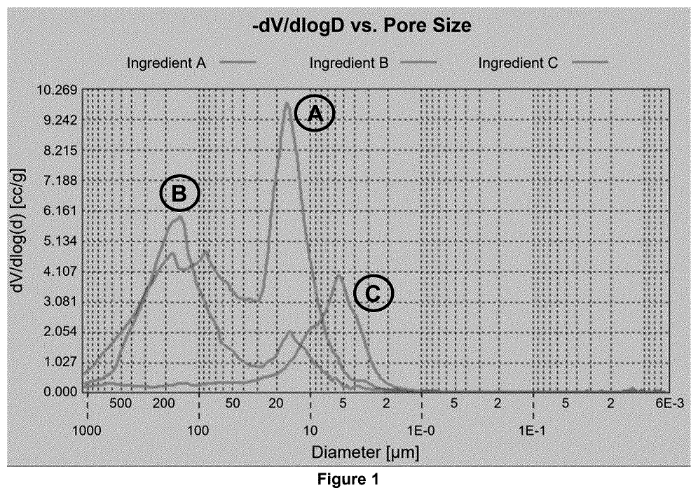

FIG. 2 shows the thermogravimetric analysis curve of ingredient A, that shows the effect of temperature versus the weight of the sample. The weight is expressed in terms of the percentage of the sample that remains, versus the weight at the start of the experiment, at a given temperature/time. second y axis on the graph which presents the data for the first derivative of the TGA curve. This is known as the Derivative Thermogravimetric (DTG) curve and represents the rate of change of mass with respect to temperature (e.g., % mass loss per degree Celsius).

FIG. 3 shows the thermogravimetric analysis curve of ingredient B, that shows the effect of temperature versus the weight of the sample. The weight is expressed in terms of the percentage of the sample that remains, versus the weight at the start of the experiment, at a given temperature/time. Second y axis on the graph which presents the data for the first derivative of the TGA curve. This is known as the Derivative Thermogravimetric (DTG) curve and represents the rate of change of mass with respect to temperature (e.g., % mass loss per degree Celsius).

FIG. 4 shows the thermogravimetric analysis curve of ingredient C, that shows the effect of temperature versus the weight of the sample. The weight is expressed in terms of the percentage of the sample that remains, versus the weight at the start of the experiment, at a given temperature/time. second y axis on the graph which presents the data for the first derivative of the TGA curve. This is known as the Derivative Thermogravimetric (DTG) curve and represents the rate of change of mass with respect to temperature (e.g., % mass loss per degree Celsius).

FIG. 5 shows beef, chicken, green peas, and soya beans used in the comparison experiments.

### DETAILED DESCRIPTION OF THE INVENTION

The invention is described in detail in the following. It is to be understood that all the disclosed features can be combined with each other, unless explicitly indicated to the contrary. In particular, features disclosed in different embodiments can be combined with each other unless explicitly indicated that such a combination is not possible.

The mycelium ingredients A, B, and C of the present invention possess unique organoleptic and biological/physical/chemical properties.

In particular, the present invention provides an edible mycelium ingredient comprising non-differentiated mycelium biomass having an elemental composition of a C:N ratio of mycelium ranging between 2 and 12 (preferably 2 and 8, more preferably 2 and 6) and characterized by EUC of at least 500 g MSG/100 g. As it is to be understood herein, the non-differentiated mycelium biomass can be obtained e.g. in the course of submerged fermentation.

This particularly preferred edible mycelium ingredient of the present invention may also be referred to as ingredient C.

It is preferred that EUC is defined as:

$$E U C = \sum aibi + 1 \; 218 ( \sum aibi ) ( \sum ajbj ) ,$$

wherein the EUC of the sample is expressed in g MSG/100 g, ai is the concentration (g/100 g) of each umami amino acid Asp or Glu, aj is the concentration (g/100 g) of each umami 5′-nucleotide 5′-IMP, 5′-GMP, or 5′-AMP, bi is the relative umami concentration (RUC) for each umami amino acid to MSG, defined as 1 for Glu and 0.077 for Asp and bj is the RUC for each umami 5′-nucleotide defined as 1 for 5′-IMP, 2.3 for 5′-GMP and 0.18 for 5′-AMP.

In an alternative embodiment, EUC is defined as:

$$E U C = \sum aibi + 1 \; 218 ( \sum aibi ) ( \sum ajbj ) ,$$

wherein the EUC of the sample is expressed in g MSG/100 g, ai is the concentration (g/100 g) of each umami amino acid Asp or Glu, aj is the concentration (g/100 g) of each umami 5′-nucleotide 5′-IMP, 5′-GMP, 5′-XMP or 5′-AMP, bi is the relative umami concentration (RUC) for each umami amino acid to MSG, defined as 1 for Glu and 0.077 for Asp and bj is the RUC for each umami 5′-nucleotide defined as 1 for 5′-IMP, 2.3 for 5′-GMP, 0.61 for 5′-XMP and 0.18 for 5′-AMP.

Accordingly, the present invention provides an edible mycelium ingredient characterized by a specific very high EUC value, in other words an ingredient with very strong umami taste properties. The ingredient of the present invention has EUC of at least 500 g MSG/100 g, preferably of at least 1000 g MSG/100 g, more preferably of at least 1500 g MSG/100 g, even more preferably of at least 2000 g MSG/100 g.

The ingredient of the present invention is further characterized by content of 5′-AMP of from 3.5 to 10.0 g/kg and/or by a content of 5′-GMP of from 3.5 to 10.0 g/kg. Preferably, the ingredient of the present invention is characterized by content of 5′-AMP of from 4.5 to 6.5 g/kg and/or by a content of 5′-GMP of from 4.5 to 6.5 g/kg. Accordingly to the present inventors, the ingredient of the present invention is preferably substantially free of 5′-IMP. In other words, the ingredient of the invention does not comprise 5′-IMP.

As it is to be understood herein, preferably the organoleptic or morphological property is selected from taste attributes, smell attributes, aroma attributes, mouthfeel attributes, texture attributes, consistency, edibility, and colour. While this list is to be construed as exemplary and preferred, it should not be construed as limiting. The skilled person shall be in a position to extend the method by including further organoleptic or morphological property or properties.

As preferably understood herein, taste attributes and smell attributes can be determined by tasting panels, composed of individuals that assess the taste and/or the smell of their provided samples. Preferably, tasting and smell panel are performed in parallel on several samples, and include certain reference samples for normalization of the assessment. Accordingly, in an exemplary way of executing a tasting panel, each trained panelist is blindfolded and successively receives a sample. They define the sensory attributes they recognize in the samples, discuss the attributes together and choose common attributes that every panelist can associate to the same taste and aroma of the samples and reference samples to compare them. A second session is then started, and the panelists have to evaluate the samples according to the chosen attributes and put a score, e.g., between 0 and 5, for each attribute. The session may be repeated on different days to increase statistical relevance of data and average of scoring may be calculated and plotted on a spider web.

Preferably, mouthfeel attributes and texture attributes may also be determined by a particular panel.

The colour as referred to herein is preferably determined using the RGB system and a colour analyzer at several positions, e.g., 20 different positions, on the samples. The mean values of these measurements are then used to compare the colour of the product. Preferably, a calibrated image capturing device is used to determine the colour.

In the invention, the nutritional property is preferably selected from sugar content, amino acid composition, content of metabolites, mineral content, vitamin content, carbohydrate content, fiber content, fatty acid content, lipid content and protein content, functional substances content and/or the C, H, N, O, S content. While this list is to be construed as exemplary and preferred, it should not be construed as limiting. The skilled person shall be in position to extend the method by including further nutritional property or properties.

As it is to be understood herein, in the present invention sugar content or carbohydrate content preferably refers to the % w/w or wt. % of content of the biomass or the product of the invention, preferably expressed with regard to the dry mass of said biomass or said product. The information on the sugar content may also include further details, e.g., content of complex and simple carbohydrates, including the breakdown with regard to pentoses or hexoses. The content of different types of sugars/carbohydrates, as referred to herein, may also be expressed in % w/w or wt. % with regard to the total sugar/carbohydrate content of the product or the biomass.

As understood herein, unless indicated to the contrary, the terms % w/w and wt. % are meant to be interchangeable.

As it is to be understood herein, in the present invention, the amino acid composition preferably refers to % w/w content of each amino acid with regard to the total amino acid content in the biomass or the product of the present invention. As known to the skilled person, in certain methods of amino acid analysis it is not possible to distinguish between aspartate and asparagine, as well as between glutamate and glutamine, due to the hydrolysis conditions employed in the process. Accordingly, the content of Asp/Asn as well as the content of Glu/Gln shall be expressed as total content of the two amino acids in each of these pairs.

As it is to be understood herein, the umami amino acids are aspartic acid (aspartate) and glutamic acid (glutamate) amino acids that contribute to the flavor, whereas branched-chain amino acids (BCAAs) are a group of three essential amino acids: leucine, isoleucine and valine, which are responsible for muscle growth, exercise performance, weight loss and fatigue reduction.

Umami or savory taste, is also defined by the 5-ribonucleotides or 5′NMP, including the following 5′-nucleotides (5′NMP): 5′-inosine monophosphate (IMP), 5′-guanosine monophosphate (GMP) and 5′-adenosine monophosphate (AMP), preferably expressed in g/kg of mycelial ingredient.

5′-GMP is known to give a meaty flavor and is a much stronger flavor enhancer than monosodium glutamate (MSG) (J. H. Litchfield, Morel mushroom mycelium as a food flavoring material Biotechnology and Bioengineering, 9 (1967), pp. 289-304). It was also reported that a synergistic effect of umami 5′-nucleotides and umami amino acids may exist to greatly increase the umami taste of mushrooms (Yamaguchi S, Yoshikawa T, Ikeda S, Ninomiya T. Measurement of the relative taste intensity of some a-amino acid and 5′-nucleotides. J Food Sci. 1971; 36:846-49.). Yamaguchi (1967) then derived this synergistic effect of 5′-IMP or 5′-GMP versus MSG in a linear relationship, expressed as follows,

$$E U C = A + 1218 ( A ) ( N )$$

wherein the EUC (equivalent umami concentration) is the equivalent concentration of monosodium glutamate (MSG), that is a measurement that considers the relative taste intensity of the umami amino acids and 5′-nucleotides, because the intensity of the umami amino acids taste and flavour nucleotides was shown to be proportional to that of MSG versus IMP or GMP, respectively. EUC is expressed in g MSG per 100 g dry weight, A and N are the concentrations of amino acids and nucleotides expressed in terms of the concentrations of MSG and GMP in solution, respectively. 1218 is a positive constant when IMP is used based on the concentration g/100 g used. This constant is 2800 when GMP is used for normalization, and both would lead the same result within an acceptable standard deviation (Yamaguchi S, Yoshikawa T, Ikeda S, Ninomiya T. Measurement of the relative taste intensity of some a-amino acid and 5′-nucleotides. J Food Sci. 1971; 36:846-49.). In this case, both amino acids and nucleotides are considered to quantify the final taste or an equivalent umami concentration in terms of an equivalent concentration of MSG.

In further details, the calculation is further expressed as: EUC=Σaibi+1218 (Σaibi) (Σajbj), wherein the EUC of the sample is expressed in g MSG/100 g, ai is the concentration (g/100 g) of each umami amino acid (Asp or Glu), aj is the concentration (g/100 g) of each umami 5′-nucleotide (5′-IMP, 5′-GMP, 5′-AMP), bi is the relative umami concentration (RUC) for each umami amino acid to MSG (Glu=1 and Asp=0.077) as reported in the literature; bj is the RUC for each umami 5′-nucleotide (5′-IMP=1; 5′-GMP=2.3; 5′-AMP=0.18) and 1218 is a synergistic constant based on the concentration (g/100 g) used. EUC is a useful parameter to benchmark umami tastes with concrete values objectively.

As it is to be understood herein, in the present invention, nucleic acid content refers to the total nucleic acid content (DNA and RNA) of the biomass or the product as defined herein, preferably referred to in % w/w or wt. % of dry mass of the biomass or of the product.

As it is to be understood herein, in the present invention the content of metabolites refers to an amount of each metabolite in the biomass or in the product, expressed in mg per g of biomass or product, respectively. For example, metabolite content may refer to any of the metabolites selected from those known to the skilled person. Typically, metabolites refer to products of the metabolism of fungal species, as referred to herein. Exemplary metabolites include alcohols, amino acids, nucleotides, antioxidants, organic acids (e.g., acetic acid, lactic acid), polyols (e.g., glycerol) and vitamins. However, this list is not meant to be construed as limiting.

As it is to be understood herein in the present invention the mineral content refers to the content of any of the minerals that are considered essential in human nutrition, which, for each of the minerals, may be expressed in mg/kg of the biomass or the product, referring to the dry mass of said biomass or said product. Preferably, minerals as referred to herein are selected from calcium, phosphorus, potassium, sodium, chloride, magnesium, iron, zinc, iodine, chromium, copper, fluoride, molybdenum, manganese, and selenium.

As it is to be understood herein, in the present invention the vitamin content preferably refers to content of a particular vitamin, referred to in μg/kg of dry biomass or dry product. Vitamins are known to the skilled person and include vitamin A, vitamin B₁₂, vitamin B₁, Vitamin B₃ vitamin B₆, vitamin C, vitamin D, vitamin E, vitamin K and vitamin O, among others.

It is understood that the final fungal-derived product based on the disclosed mycelial ingredients may include the supernatant obtainable in the process of the production of the mycelial ingredients of the present invention. Accordingly, the final fungal-derived product based on the disclosed mycelial ingredients may be said supernatant itself, or the biomass (i.e., said mycelial ingredient) or a combination thereof or any related extracts from each individual product or a combination thereof. Therefore, the fungal ingredients may be applicable to at least one form of these products, as it would be apparent to the skilled person.

As it is to be understood herein, in the present invention, the fungal composition preferably relates to species/strain composition of the biomass or the product derived therefrom i.e. from the disclosed mycelial ingredients of this invention.

As it is to be understood herein, in the present invention, the biomass dry weight is the weight of the obtained biomass upon dehydration/water removal, preferably after washing off the residual medium, preferably extrapolated down to 0% w/w water content.

As it is to be understood herein, preferably whenever a reference is made to any content or any ratio determined “on a dry basis” or “on a dry weight basis”, it refers to a material upon dehydration/water removal, preferably after washing off the residual medium, preferably extrapolated down to 0% w/w water content.

As it is to be understood herein, in the present invention the fiber content preferably refers to % w/w content of dietary fiber in the dry biomass or the dry product.

As it is to be understood herein, in the present invention the protein content preferably refers to % w/w content of the protein in the dry biomass or the dry product. Preferably the protein content measured refers to the protein content determined by the Kjeldahl method.

As it is to be understood herein, in the present invention the fatty acid content preferably refers to % w/w content of fatty acid in the dry biomass or the dry product.

As it is to be understood herein, in the present invention the lipid content preferably refers to % w/w content of lipids in the dry biomass or the dry product.

It is to be understood that the terms mycelial ingredient or mushroom mycelium ingredient or mycelium ingredient or fungal ingredient or mycelium/mycelial biomass are equivalent.

As it is to be understood herein, in the methods of the present invention, product titer preferably refers to the concentration of the obtained product, preferably expressed in g/L. Herein, the term product preferably refers to a fungal biomass, a fungal metabolite, i.e., compounds obtainable from the mycelium, a colorant, a nutraceutical, an active compound, an enzyme, or a cosmetic product.

As it is to be understood herein, in the methods of the present invention, cultivation or fermentation conditions include data necessary to repeat the cultivation experiments, i.e., temperature, CO₂ content/exhaust/production or other exhausts of volatile gases, agitation, humidity, pH, dissolved oxygen concentration, dissolved CO₂ concentration, etc. This list is not meant to be limiting, as cultivation conditions are apparent to the skilled person.

As it is to be understood herein, in the methods of the present invention, the metabolic behavior preferably comprises transcriptome information, metabolome information, proteome information, secretome information, and/or fluxome information. It is to be understood that the secretome preferably includes the information on structures and/or amounts of compounds produced by the fungal biomass and secreted outside the fungal cells, e.g., metabolite/protein that can be secreted in the fermentation broth by an organism. As understood by the skilled person, it could include valuable compounds that can be used for products such as vitamins, enzymes, pigments or mycelium-derived functional or active compounds. Accordingly, the information on secretome could inform the efforts to produce secreted compounds in a process involving fungal biomass.

Preferably, the molecule and metabolite information concerns metabolites present in or obtainable from a particular fungus. Accordingly, metabolites should preferably be understood as every node of the metabolism pathway map of specific species under investigation. Information on the structure and content of particular metabolites is preferably included herein.

As it is to be understood herein in the present invention, the fermentation medium preferably comprises at least one fungal strain, optionally other microbes to be co-cultured with the fungal strain (preferably including algae, bacteria, plant cells, archaea cells, animal cells, fat cells or a combination thereof), and/or a side stream from the agrifood industry.

As to be understood herein, chitin is a polysaccharide consisting of connected N-acetylglucosamine subunits with the chemical formula of (C8H1305N) n, with n being the number of subunits. It is to be understood that n is a natural number. Chitin and its degradation products, via the enzyme chitinase present in humans and other mammals, are sensed in the skin, lungs, and digestive tract, triggering an immune response with a potential directed towards the fight of parasites. In addition, chitin is usually used as a food additive to improve flavor and as an emulsifier. It also has an anti-inflammatory property, reduces cholesterol and is beneficial for weight loss and blood pressure. Chitin can also be used to produce biomaterials or biodegradable packaging materials, also for the food industry or other industries. It could also be a compound used as a sausage casing as chitin could be extracted from mushrooms or mycelium.

Preferably, production of functional compounds includes information of compounds produced by the mycelium. Preferably, said functional compounds, which may also be referred to as active compounds, preferably refer to any substance with a beneficial (documented or proven) effect on a biological function, are preferably mycelium-derived active compounds selected from ergothioneine, lovastatin, ergosterol, resveratrol, glutathione, eritadenine, lentinan, and Concanavalin A. However, this list is not meant to be construed as particularly limited and further compounds produced in the mycelium, as recognized by the skilled person, may also be included. Exemplary compounds originating from Pleurotus ostreatus have been recently reviewed (Mishra et al., Int J Biol Macromol, 2021, 182, 1628-1637).

Ergothioneine is a sulfur-based amino acid that is found mainly in mushrooms and in red/black beans. It is known for its antioxidant and anti-inflammatory properties to prevent chronic diseases of aging, such as cardiac or brain related diseases and could protect against cell and tissue damage in the body. It is sometimes considered as a longevity vitamin, for its beneficial effect. Preferably, ergothioneine is a compound of formula:

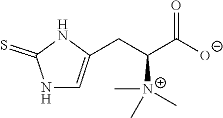

or its salt.

Oyster mushrooms are a major source of nutraceuticals and known to have immense therapeutic properties, such as the mediation of anti-tumor, anti-angiogenesis immunomodulatory, antioxidant, and anti-diabetic roles. Such macromolecules are like β-glucan, a-glucan, ergosterol, linoleic acid etc. It has also been shown that the intake of ergosterol may increase the vitamin D concentrations in serum and liver.

Polyphenols are the most antioxidants consumed in human diets. The total phenolic content (TPC) refers to phenolic compounds having redox properties responsible for antioxidant activity and known to have potential beneficial effects on human health. Similarly phytonutrients like flavonoids also have anti-inflammatory properties and they can act as antioxidants and protect cells from oxidative damage leading to diseases.

The total phenolic content is measured by a Folin-Ciocalteu assay and is preferably expressed here in mg GAE per g biomass, where in GAE is the gallic acid equivalents. The total flavonoid content (TFC) was measured by the aluminum chloride colorimetric assay and preferably it is expressed here in mg QE per g biomass, wherein QE is the Quercetin equivalent. The polyphenols further profiling was measured via a high-performance liquid chromatography with diode-array detection (HPLC-DAD) to examine the content of the biomass in potential polyphenols present, such as, catechin, vanillin, quercetin, chlorogenic acid, 3,4-dihydroxybenzoic acid (Protocatechuic acid), salicylic acid, p-coumaric acid, syringic acid, vanillic acid, caffeic acid, ferulic acid, and gallic acid. The concentration of the following substances is preferably expressed in mg/g. These methods are well-known methods for a person skilled in the art.

In particular, catechins are natural antioxidants that help prevent cell damage and provide other benefits. Quercetin is a plant pigment (flavonoids) and may help in reducing swelling, killing cancer cells and help prevent heart diseases. Chlorogenic acids are mainly found in coffee and other foods and have been extensively investigated in neurodegenerative diseases because of its anti-inflammatory activity. 3,4-Dihydroxybenzoic acid (protocatechuic acid) is usually found in green tea and known to have mixed effects on normal and cancer cells. protocatechuic acid is considered as an active component of some traditional Chinese herbal medicines such as Cibotium barometz (L.) (Functional Foods in Health and Disease, vol. 7, pp. 232-244, 2011.) For example, Acai oil sourced from the Acai palm fruit (Euterpe oleracea) is rich in protocatechuic acid (630 mg/kg) (Journal of Agricultural and Food Chemistry, vol. 56, no. 12, pp. 4631-4636, 2008).

As it is to be understood herein, C:N ratio (mycelium) preferably relates to the ratio between contents of carbon to nitrogen in the mycelial ingredient, whereas C:N ratio (medium) preferably relates to the ratio between contents of carbon to nitrogen in the fermentation medium, i.e., in the fermentation broth. The ratio is preferably herein understood as w/w ratio.

As it is to be understood herein, the degree of complexity of the fermentation medium may be categorized in three categories: a synthetic medium, a defined medium, and a complex medium.

A synthetic medium is preferably a medium, where no complex compounds exist i.e., concentrations of all the components are well-known. For example, a medium without a complex nitrogen source is a synthetic medium.

A defined medium is preferably a medium comprising at most one complex compound (e.g., yeast extract), with potential compounds having unknown concentrations.

A complex medium is preferably a fully undefined composition with more than one complex compound and unknown substances or natural extracts having unknown concentrations (e.g., compounds extracted from agriculture sidestreams, or waste streams from the food industry). Preferably, the complex medium is defined through the process of its production.

Brunauer-Emmett-Teller (BET) surface area is a method used to characterize porous and finely-dispersed solids via gas adsorption. During the gas adsorption (according to DIN-ISO 9277 respectively DIN 66131) the specific surface of solid matters is determined by default with nitrogen adsorption at 77.4 K using the BET method. The evaluation takes place in the general area of validity of the BET method of p/p0=0.05-0.3 respectively in the stated relative pressure area. For the determination of very small surfaces the krypton adsorption (at 77.4 K) is used. Because the expected surface areas are quite low, Krypton has been used, because Krypton is the suitable adsorbate for measuring low surface areas. The specific surface area is expressed in m²/g.

The Median pore diameter means that 50% of the pore-volume comes from pores larger than the median pore diameter and the other 50% of the pore-volume comes from pores smaller than the median pore diameter.

The specific pore volume can be determined from nitrogen adsorption measurements if the adsorbent is meso- or microporous. For macroporous adsorbents with pore diameters above 1000 Å, the pore volume can be determined by mercury porosimetry measurements by integrating the pressure-volume curve. Method is based on the Washburn-Equation, which describes the relationship between pore diameters and applied pressure to a non-wetting liquid like mercury.

The fermentation process, which may preferably be a biochemical process, is not meant to be particularly limited and as recognizable to the skilled person, any process that involves microorganisms could be encompassed by the present invention. Particularly preferred are cultivation process (e.g., production of biomass, in particular of fungal biomass) and bioproduction processes (e.g., expression of enzymes in the microbial culture, bioproduction of ethanol, production of intracellular or extracellular compounds, e.g., colourants, flavoring compounds, antioxidants, enzymes, moisturizing compounds, and similar processes).

Accordingly, submerged fermentation in the method of the present invention can be operated as a batch, a fed-batch or a continuous process. These three main methods of fermentation are known to the skilled person and differ by outflow and inflow of material from/to the fermentation vessel.

The batch processes are characterized by lack of inflow of material into the fermentation vessel. In a batch process, all nutrients are provided at the beginning of the cultivation, without adding any more in the subsequent bioprocess. During the entire bioprocess, no additional nutrients are added except for gases, acids, and bases. The bioprocess then lasts until the nutrients are consumed. This strategy is suitable for rapid experiments such as strain characterization or the optimization of nutrient medium. The disadvantage of this convenient method is that the biomass and product yields are limited. Since the carbon source and/or oxygen transfer are usually the limiting factor, the microorganisms are not in the exponential growth phase for a long time. After the end of a bioprocess run in batch mode, only the biomass or medium is harvested and appropriately processed to obtain the desired product. From the bioreactor point of view, the process is repeatedly interrupted by cleaning and sterilization steps, and the biomass is only produced in stages.

In the fed-batch process, substrate, nutrients, and other substances may be added into the fermentation vessel, to extend the possible culture time or increase the yield, among others. The advantage of feeding during cultivation is that it allows to achieve higher product quantities overall. Under specific growth conditions, the microorganisms and/or cells constantly double and therefore follow an exponential growth curve. Therefore, in certain embodiments the feed rate may be increased exponentially as well. Generally, the substrate is pumped from the supply bottle into the culture vessel, for example through a silicone tube. The user can either manually set the feed at any time (linear, exponential, pulse-wise), or add nutrients when specific conditions are met, such as when a certain biomass concentration is reached or when a nutrient is depleted. The fed-batch process offers a wide range of control strategies and is also suitable for highly specialized applications. However, it may increase the processing time and potentially leads to inhibition through the accumulation of toxic by-products.

In the method of the present invention, submerged fermentation is preferably operated as a continuous process. After a batch growth phase, an equilibrium is established with respect to a particular component (also called steady-state). Under these conditions, as much fresh culture medium is added, as it is removed (chemostat). These bioprocesses are referred to as continuous cultures, and are particularly suitable when an excess of nutrients would result in inhibition due to e.g., acid or ethanol build up or excessive heating. After reaching steady-state, it is understood that a continuous-mode is constantly operated in the exponential growth phase, wherein cells are maintained at a constant concentration. Transient state is the state before reaching constant stead-state conditions for continuous-mode and it is in fact similar to the start of a batch-mode operation. Other advantages of this method include reduced product inhibition and an improved space-time yield. When medium is removed, cells are harvested, which is why the inflow and outflow rates must be less than the doubling time of the microorganisms. Alternatively, the cells can be retained in a wide variety of ways (for example, in a spin filter), which is called perfusion. In a continuous process, the space-time yield of the bioreactor can be even further improved compared to that of a fed-batch process. However, the long cultivation period also increases the risk of contamination and long-term changes in the cultures. The three most common types of continuous culture are chemostat (the rate of addition of a single growth-limiting substrate controls cell multiplication), turbidostat (an indirect measurement of cell numbers-turbidity or optical density-which needs an additional sensor but is driven by real-time feedback, controls addition and removal of liquid), and perfusion (this type of continuous bioprocessing mode is based on either retaining the cells in the bioreactor or recycling the cells back to the bioreactor; fresh medium is provided and cell-free supernatant gets removed at the same rate).

In one embodiment of the present invention, in the method of the present invention the submerged fermentation is not operated as a continuous process.

In a preferred embodiment the edible fibrous mycelium mass is derived from submerged fermentation. The submerged fermentation allows mycelia to grow without the need for a substrate, which supports them structurally like in solid-state fermentation. Additionally, growth rates are higher as nutrients can be transported to all points of the mycelia and it is easier to maintain sterile conditions. Depending on the fermentation conditions, the mycelia have the possibility to grow in the submerged fermentation into either pellets or threads. This structure is maintained at harvest, giving different texture properties to the resulting products, such as cheese products if processed mildly.

Furthermore, submerged fermentation provides the additional advantage that a clean product is obtained that is not contaminated with the residues of the solid substrates that can bring bad taste or allergies afterwards, in particular because washing of the mycelium grown in submerged fermentation can be easily achieved. Similarly, byproducts from fermentation such as acids or alcohols that can alter the taste as well can also be easily removed.

In a submerged fermentation, it is also easier to better control fermentation conditions such as pH and oxygen content. Therefore, it is easier to enable the maximal growth of the mycelium compared to substrate limitation in solid-state fermentation. Thus, a more homogenous product composition can be obtained, which also enables reduction of the variation from batch to batch. Furthermore, the material obtained by submerged fermentation is more malleable and can be formed into any kind of shape. For example, mycelium can be homogenized into a liquid that can be fermented, which is particularly advantageous if the mycelium is used to produce a dairy substitute product. Mycelium obtained from solid-state fermentation is usually dried and therefore needs to be resuspended in water to get a milk like substance.

Furthermore, in submerged fermentation, spore formation or fruiting body formation that could lead to toxin production can be prevented easily and efficiently as the fermentation media can be agitated. It is further apparent to the skilled person that if the growth is performed in the submerged fermentation, preferably no differentiation of mycelium to spores and/or fruiting bodies occurs. Thus, it is apparent to the skilled person that the mycelium mass derived from submerged fermentation may also be referred to as non-differentiated mycelium biomass. The skilled person would recognize further advantages of using submerged fermentation, e.g., a better control over texture and composition of biomass compared to solid state fermentation.

Furthermore, submerged fermentation methods are easier to scale up, as a second culture can be inoculated with the first culture, as both cultures are in a liquid state. This is not easily possible in a solid-state fermentation, as the first culture cannot flow. This disadvantage can in part be remedied by using a rotating drum reactor, wherein the reactor is rotated to mix the solids and the mycelium. Drums reactors, however, are known for their huge problem of heat transfer during fermentation that leads to different temperatures in the reactor. There may be parts of the fermentation volume that are overheated at some point because heat cannot be removed properly, or parts of the fermentation volume, especially at the fermentation start, that are underheated. In submerged fermentation, the heat transfer will be more efficient as liquid is a good medium for transferring heat, leading to more homogeneous heat distribution and thus a more controllable, more efficient fermentation process.

Accordingly, preferably as encompassed by the present invention, the process is performed with at least one fungal species. The at least one fungal species may be combined with edible fungi. The at least one fungal species may be combined with edible fungi, algae, bacteria, plant cells, archaea cells, animal cells, fat cells or a combination thereof.

Preferably, in one embodiment, edible fungi currently discovered, or which will be discovered and the co-cultivation or co-fermentation of such edible fungi with each other is used. In another embodiment, the previous embodiment is further combined with the usage of algae or bacteria or plant or archaea or animal cells/fat cells or a combination thereof.

Preferably, the at least one fungal species is selected from Basidiomycota, Ascomycota, Pezizomycotina, Agaromycotina, Pezizomycetes, Agaricomycetes, Sordariomycetes, Pezizales, Boletales, Cantharellales, Agaricales, Polyporales, Russulales, Auriculariales, Hypocreales, Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, and Fistulinacea.

In one embodiment, edible fungi currently discovered, or which will be discovered and the co-cultivation or co-fermentation of such edible fungi with each other is used. In another embodiment, the previous embodiment is further combined with the usage of algae or bacteria or plant or archaea or animal cells/fat cells or a combination thereof.

According to the present invention, the at least one fungal strain can be selected from the division Basidiomycota. Preferably, the at least one fungal strain selected from Basidiomycota can be a fungal strain selected from the subdivision Agaromycotina. As defined herein, a fungal strain selected from the subdivision Agaromycotina can be a fungal strain selected from the class Agaricomycetes. Preferably, a fungal strain selected from Agaricomycetes can be a fungal strain selected from the order Agaricales, Auriculariales, Boletales, Cantharellales, Polyporales, and Russulales.

When the fungal strain is selected from the order Agaricales, the fungal strain is preferably selected from the family Agaricaceae, Fistulinaceae, Lyophyllaceae, Marasmiaceae, Omphalotaceae, Physalacriaceae, Pleurotaceae, Schizophyllaceae, Strophariaceae, and Tricholomataceae.

The fungal strain selected from Agaricaceae can be Agaricus bisporus or Agaricus blazei, more preferably Agaricus bisporus.

The fungal strain selected from Fistulinaceae is preferably Fistulina hepatica.

The fungal strain selected from Lyophyllaceae is preferably Calocybe indica.

The fungal strain selected from Marasmiaceae is preferably Lentinula edodes.

The fungal strain selected from Omphalotaceae is preferably Calvatia gigantea.

The fungal strain selected from Physalacriaceae is preferably Flammulina velutipes.

More preferably, the at least one fungal strain selected from Agaricales can be a fungal strain selected from Pleurotaceae. Even more preferably, the at least one fungal strain of the present invention is a fungal strain selected from Pleurotus pulmonarius, Pleurotus ostreatus, Pleurotus citrinopileatus, Pleurotus florida, Pleurotus eunosmus, Pleurotus columbinus, Pleurotus ferulae, Pleurotus salmoneo-stramineus and Pleurotus salmoneostramineus, even more preferably selected from Pleurotus pulmonarius or Pleurotus ostreatus, most preferably Pleurotus pulmonarius.

The fungal strain selected from Schizophyllaceae is preferably Schizophyllum commune. The fungal strain selected from Strophariaceae is preferably a fungal strain selected from Agrocybe aegerita and Hypholoma capnoides.

The fungal strain selected from Tricholomataceae is preferably a fungal strain selected from Hypsizygus tesselatus and Clitocybe nuda.

Alternatively, a fungal strain selected from Agaricomycetes can be a fungal strain selected from the order Auriculariales, more preferably a fungal strain selected from the family Auriculariaceae. Preferably, a fungal strain selected from Auriculariaceae is Auricularia auricula-judae.

When the fungal strain is selected from the order Boletales, the fungal strain is preferably selected from the family Boletaceae and Sclerodermataceae. The fungal strain selected from Boletaceae is preferably Boletus edulis.

When the fungal strain is selected from the order Cantharellales, the fungal strain is preferably selected from the family Cantharellaceae and Hydnaceae. The fungal strain selected from Cantharellaceae can be Cantharellus cibarius. The fungal strain selected from Hydnaceae can be Hydnum repandum.

When the fungal strain is selected from the order Polyporales, the fungal strain is preferably selected from the family Ganodermataceae, Meripilaceae, Meruliaceae, Polyporaceae, and Sparassidaceae, more preferably from Ganodermataceae, Meripilaceae, Polyporaceae, and Sparassidaceae.

The fungal strain selected from Meripilaceae is preferably Grifola frondosa. The fungal strain selected from Polyporaceae can be from Polyporus umbellatus and Laetiporus sulphureus (L. sulphureus). The fungal strain selected from Sparassidaceae can be Sparassis crispa. The fungal strain selected from Meruliaceae is preferably selected from B. adusta and B. fumosa.

When the fungal strain is selected from the order Russulales, the fungal strain can be selected from the family Bondarzewiaceae and Hericiaceae. Preferably, a fungal strain selected from Russulales is a fungal strain selected from Hericiaceae, preferably selected from Hericium erinaceus and Hericium coralloides. The fungal strain selected from Bondarzewiaceae can be Bondarzewia berkeleyi.

According to the present invention, the at least one fungal strain can be selected from the division Ascomycota. Preferably, the at least one fungal strain selected from Ascomycota can be a fungal strain selected from the subdivision Pezizomycotina.

The fungal strain selected from Pezizomycotina can be selected from the class Pezizomycetes. Preferably, the fungal strain selected from Pezizomycetes can be a fungal strain selected from the order Pezizales. Preferably, the fungal strain selected from Pezizales can be selected from the family Morchellaceae and Tuberaceae.

In preferred embodiments, the mycelium is not derived from Sordoriomycetes, in particular is not derived from the genus Neurospora, for example from Neurospora crassa or from the genus Fusarium, for example from Fusarium venenatum.

Preferably, the fungal strain selected from Morchellaceae is Morchella esculenta, Morchella angusticeps, Morchella deliciosa, Morchella sceptrifomtis, Morchella steppicola, Morchella puncripes, Morchella rufobrunnea, Morchella importuna, Morchella Jaurentinaa, or Morchella purpumscens, preferably Morchella esculenta, Morchella angusticeps or Morchella deliciosa.

Preferably, the fungal strain selected from from Tuberaceae is Tuber magnatum, T. estivum, T. uncinatum, T. indicum, T. rufum or T. melanosporum, more preferably T. melanosporum and T. magnatum.

Alternatively, the at least one fungal strain selected from Ascomycota can be a fungal strain selected from the class Sordariomycetes.

Preferably, the fungal strain selected from Sordariomycetes can be a fungal strain selected from the order Hypocreales.

The fungal strain selected from Hypocreales can be a fungal strain selected from the family Cordycipitaceae. The fungal strain selected from Cordycipitaceae can be a fungal strain selected from Cordyceps militaris and Cordyceps sinensis.

Alternatively, a fungal strain selected from Hypocreales can be a fungal strain selected from the family Nectriaceae. The fungal strain selected from Nectriaceae can be a Fusarium strain, for example Fusarium venenatum.

In another embodiment, the fungal strain selected from Sordariomycetes can be a fungal strain selected from the family Sordariaceae. The fungal strain selected from Sordariaceae can be a Neurospora strain, for example Neurospora crassa.

Preferably, the edible fibrous mycelium mass is obtained from at least one fungal strain selected from Basidiomycota, Ascomycota, Pezizomycotina, Agaromycotina, Pezizomycetes, Agaricomycetes, Sordariomycetes, Pezizales, Boletales, Cantharellales, Agaricales, Polyporales, Russulales, Auriculariales, Hypocreales, Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, and Fistulinacea.

More preferably, the edible fibrous mycelium mass is obtained from at least one fungal strain selected from Basidiomycota, Ascomycota, Pezizomycotina, Agaromycotina, Agaricomycetes, Pezizales, Boletales, Cantharellales, Agaricales, Polyporales, Russulales, Auriculariales, Hypocreales, Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, and Fistulinacea.

Even more preferably, the mycelium mass is obtained from Pleurotus pulmonarius, Pleurotus ostreatus, Pleurotus florida, Pleurotus citrinopileatus, Pleurotus salmoneostramineus, Morchella esculenta, Morchella angusticeps, or Morchella deliciosa.

Even more preferably, the mycelium mass is obtained from Pleurotus pulmonarius, Pleurotus florida, Pleurotus citrinopileatus, Pleurotus salmoneostramineus, Morchella esculenta, Morchella angusticeps, or Morchella deliciosa, or Morchella rufobrunnea.

Most preferably, the mycelium mass is obtained from Pleurotus pulmonarius or Morchella rufobrunnea.

In one preferred embodiment, the mycelium mass is obtained from Pleurotus pulmonarius. In another preferred embodiment, the mycelium mass is obtained from Morchella rufobrunnea. In one preferred embodiment, the mycelium biomass comprises Pleurotus pulmonarius and/or Morchella rufobrunnea. In one further preferred embodiment, the mycelium biomass comprises Pleurotus pulmonarius and Morchella rufobrunnea.

In one embodiment, the mycelium biomass is obtained from L. sulphureus or B. adusta.

In a separate embodiment, the edible fibrous mycelium mass is obtained from at least one fungal strain selected from Basidiomycota, Ascomycota, Hymenochaetaceae, Agaricomycetes, Sordariomycetes, Tremellomycetes, wherein the preferable species herein are from at least one fungal species selected from Cordyceps spp., Inonotus spp., Grifola spp., Pleurotus spp., Ganoderma spp., Lentinula spp., Tremella spp., Trametes spp., Lepista spp., Tricholoma spp., Aspergillus spp., and/or Panus spp. Accordingly and preferably, the at least one fungal strain is a strain that produces ergothioneine.

Typically, a constant temperature is maintained throughout the process, which as known to the skilled person may be selected for optimal growth of a particular fungal strain. For example, in the case of P. ostreatus cultivation is preferably performed at a temperature of between 25 and 30° C. Further preferably, the cultivation is performed at a pH of between 3.0 and 8.5. As understood to the skilled person, selection of pH may be dependent on the fungal strain to be cultivated, or on potential contaminating strains to be excluded from growing. Further preferably, the step cultivation is performed for a time of between 12 and 240 hours.

Accordingly, the present invention is further related to a method for producing the mycelium ingredients via submerged fermentation, wherein the fermentation medium comprises of 5 to 60 g/L of carbon source, 0.1 to 60 g/L nitrogen source, 0.01-15 g/L of minerals, and 0.01-50 mg/L of vitamins. A skilled person shall be able to extend the method by adjusting the carbon source (i.e. the total added sugars) and/or other parameters to meet the requirements of the reactor configuration used (batch mode versus fed-batch mode versus continuous mode).

In the method for producing the mycelium ingredients via submerged fermentation, the fermentation medium preferably comprises of 5 to 60 g/L of carbon source, 0.1 to 60 g/L nitrogen source, 0.5-15 g/L of minerals, and 0.1-10 mg/L of vitamins. Preferably the carbon source is selected from Potato dextrose agar, Starch, Cellulose, Malt Extract, Beet molasses, Corn molasses, Sucrose, Glycerol, Glucose, Fructose, Lactose, Galactose, Xylose, Arabinose, and/or Maltose. Preferably, the nitrogen source is selected from Corn steep liquor (CSL), Yeast extract, Peptone, Ammonia, Urea, Ammonium sulfate, Ammonium chloride and/or Ammonium carbonate. Preferably the minerals are selected from Sodium selenate, Magnesium sulfate, Magnesium chloride, Iron sulfate, Iron chloride, Manganese chloride, Manganese sulfate, Zinc sulfate, Calcium sulfate, Calcium chloride, Calcium carbonate, Copper chloride, Copper sulfate, Diammonium hydrogen phosphate, Potassium dihydrogen phosphate, Disodium hydrogen phosphate, Sodium phosphate monobasic hydrate and/or Sodium chloride. Preferably the vitamins are selected from Biotin, Choline chloride, Folic acid, Myo-inositol, Niacinamide, Pantothenic acid, Pyridoxal, Riboflavin, Thiamine, Cobalamin and/or Ascorbic acid. pH control is preferably controlled via substances selected from Sodium chloride, Sodium hydroxide, Potassium hydroxide, Sulfuric acid, Phosphoric acid, Hydrochloric acid, Citric acid, Acetic acid, Hypochlorous acid. It is to be noted, that other components may be used like agar or food grade anti-foaming like edible oils or others. The skilled person shall be able to extend the method by including further substances, metabolites and further types of carbon, nitrogen, mineral, and vitamin sources.

In one embodiment, the medium is enriched with vitamin B12 (cobalamin), by adding B12 in its pure chemical form for the purpose of nutritionally supplementing the mycelium biomass. In a further embodiment, the medium is enriched with vitamin B12 between a range of 1 μg/L-200 μg/L, preferably between a range of 10 μg/L-100 μg/L. In certain embodiments, the accumulated vitamin B12 in the biomass is at least 10%, preferably at least 5% preferably at least 2.5%, preferably at least 1%, preferably at least 0.5% from the initial added amounts. In a preferred embodiment, the accumulation of vitamin B12 in the biomass is 10% at most, preferably 7.5% at most, more preferably 5% at most, more preferably 2.5% at most, preferably 1% at most, more preferably 0.5% at most.

In one embodiment, the fungal strain may be grown on a defined medium. A mycelium ingredient obtainable when grown on defined medium, as defined herein, may also be referred to as mycelium ingredient A.

Accordingly, the present invention relates to an edible mycelium ingredient (also referred to as mycelium ingredient A) comprising (preferably non-differentiated) mycelium biomass having an elemental composition of a C:N ratio of mycelium ranging between 6 and 8 and characterized by EUC of between 200 and 500 g MSG/100 g. It is preferred that the mycelium is Pleurotus pulmonarius mycelium. EUC is defined herein.

In another embodiment, the fungal strain may be grown on a synthetic medium. A mycelium ingredient obtainable when grown on synthetic medium, as defined herein, may also be referred to as mycelium ingredient B.

Accordingly, the present invention relates to an edible mycelium ingredient (also referred to as mycelium ingredient B), comprising non-differentiated mycelium biomass having an elemental composition of a C:N ratio of mycelium ranging between 8 and 12 and characterized by EUC of less than 200 g MSG/100 g. EUC is defined herein.

In one preferred embodiment, the present invention provides an edible mycelium ingredient comprising Pleurotus pulmonarius mycelium biomass (preferably non-differentiated biomass) having an elemental composition of a C:N ratio of mycelium ranging between 2 and 12 and characterized by an equivalence umami concentration (EUC) of at least 30 monosodium glutamate g MSG/100 g. EUC is as defined herein. The edible mycelium ingredient as described herein is obtainable in a method for producing the edible mycelium ingredient as described herein via submerged fermentation, comprising the step of culturing Pleurotus pulmonarius in a fermentation medium, wherein the fermentation medium as provided at the beginning of the fermentation is characterized by C:N ratio which ranges between 1 and 50, preferably 5 and 50, wherein the fermentation medium as provided at the beginning of the fermentation comprises of 5 to 60 g/L of carbon source, 0.1 to 60 g/L of nitrogen source, 0.01-15 g/L of minerals, and 0.01-50 mg/L of vitamins, characterized in that the medium includes arginine and glutamate as the only amino acids. In one embodiment, the w/w ratio of the arginine to glutamate preferably is 10:90, 20:80, 30:70, 40:60, 50:50, 60:40, 70:30, 80:20, or 90:10. This is preferably dictated by the specific EUC required for a certain mycelium ingredient or a food product comprising said mycelium ingredient. In a preferred embodiment, the w/w ratio of the overall arginine to glutamate is preferably 20:80, more preferably 50:50, even more preferably 40:60, most preferably 30:70. In one preferred embodiment, the medium includes glutamate as the only amino acid. It is to be understood that the invention further provides an edible product, preferably a meat substitute product or a dairy substitute product, comprising the edible mycelium ingredient as described herein, ranging from 1 to 99 wt. %,

Preferably, the defined medium comprises more than one amino acid and more than one vitamin, preferably each in different concentrations. In this case the biomass can have the possibility to grow on different sources. Preferably, the amino acids and vitamins are sourced from at least one complex nitrogen source, preferably selected from yeast extract, CSL and peptone, more preferably yeast extract. The carbon source is preferably sourced from dextrose, glucose, maltose, galactose or malt extract or cellulose. The carbon to nitrogen ratio (medium), i.e., C:N ratio in this defined medium ranges between 5 and 50, preferably between 10 and 25, preferably between 14 and 19. In one embodiment, the carbon to nitrogen ratio (medium), i.e., C:N ratio in this defined medium ranges 10 and 25, preferably between 16 and 18, more preferably between 16.5 and 17.5.

Preferably, the synthetic medium comprises at least one amino acid and at least one type of vitamin, wherein (1) the at least one vitamin used has an amount characterized by the ratio of said at least one vitamin used to the total amount of all vitamins present in the defined medium (taking the preferred nitrogen complex as a reference and expressing the ratio as w/w) being in the range of 0.01 to 10, preferably 0.1 to 10, more preferably 0.5 to 10 and (2) the at least one amino acid used has an amount characterized by the ratio of said at least one amino acid used to the total amount of all the amino acids present in the defined medium being in the range of 0.01 to 10, preferably 0.1 to 10, more preferably 0.5 to 10 (wherein said ratio is expressed as w/w ratio).

Preferably, the nitrogen source concentrations ranges between 0.1 to 60 g/l, more preferably between 1 to 40 g/l, even more preferably between 1 and 30 g/l, most preferably between 1 and 20 g/l. In another preferred embodiment, the nitrogen source concentrations ranges between 0.1 and 10 g/l, preferably is about 6 g/l.

The carbon to nitrogen ratio (medium), i.e., C:N ratio in this synthetic medium ranges between 1 and 50, preferably between 5 and 50, more preferably between 10 and 25, even more preferably between 16 and 23. In one embodiment, the C:N ratio in this synthetic medium ranges between 2 and 22, preferably between 15 and 22, more preferably between 20 and 25, even more preferably between 20 and 22. In an alternative embodiment, the C:N ratio in this synthetic medium ranges between 2 and 22, preferably between 2 and 16, more preferably between 2 and 14. In one embodiment the C:N ratio in this synthetic medium ranges between 1 and 5.

The at least one amino acid is preferably selected from alanine, asparagine, aspartate, arginine, tryptophan, glycine, glutamic acid, glutamine, methionine, phenylalanine, serine, valine, cystine, proline, leucine, tyrosine, threonine, isoleucine, histidine, lysine, and selenocysteine or a combination thereof.

The at least one amino acid is preferably selected from essential amino acids, selected from Phenylalanine, Valine, Tryptophan, Threonine, Isoleucine, Methionine, Histidine, Leucine, Lysine or a combination thereof.

Alternatively, this at least one amino acid is preferably selected from non-essential amino acids, selected from Alanine, Arginine, Asparagine, Aspartic Acid (or aspartate), Cysteine, Glutamic acid (or glutamate), Glutamine, Glycine, Proline, Serine, Tyrosine, Selenocysteine or a combination thereof. More preferably, this at least one amino acid is preferably selected from non-essential amino acids, selected from Alanine, Arginine, Aspartic Acid (or aspartate), Cysteine, Glutamic acid (or glutamate), Glutamine, Glycine, Proline, Serine, Tyrosine, Selenocysteine or a combination thereof.

This at least one amino acid is preferably selected from non-essential amino acids, selected from Alanine, Arginine, Cysteine, Glycine, Proline, Serine, Tyrosine, Selenocysteine or a combination thereof. This list excludes umami amino acids. It is preferred that not more than 5, preferably not more than 3, even more preferably not more than 2 types of amino acids are added to the medium. Most preferably, said at least one amino acid, which is preferably not more than 5 amino acid types, more preferably not more than 3 amino acid types, even more preferably not more than 2 amino acid types, is/are selected from Alanine, Arginine, Cysteine, Glycine, Proline, Serine, Tyrosine, and Selenocysteine.

Accordingly, the defined medium in the present invention may also be defined as a medium characterized by C:N ratio which ranges between 1 and 50, and comprising at least one amino acid selected from Alanine, Arginine, Cysteine, Glycine, Proline, Serine, Tyrosine, and Selenocysteine. It is preferred that the at least one amino acid, as defined herein, includes Arginine. It is apparent to the skilled person that certain amino acids are to be considered essential amino acids for mycelium growth, and accordingly growth of the mycelium without their addition to the medium may not be possible. The present inventors have surprisingly found that it is possible to grow the mycelium in the presence of only one non-essential amino acid, Arginine, which allows obtaining the mycelium with low umami content (as defined below and expressed by EUC parameter). The minimum amounts of amino acid added, preferably Arginine, is dictated by the desired C:N ratio of the medium. In other words, the minimum amount of amino acid added is preferably correlated to the minimum amount of carbon source required for growth, where in the carbon source required for growth is at least 5 g/L, more preferably at least 10 g/L.

Preferably, the at least one non-essential amino acid added is not more than 5 (preferably not more than 4, even more preferably not more than 3, still more preferably not more than 2) amino acids, including Arginine and at most 4 further amino acids selected from (i) non-essential amino acids selected from Alanine, Cysteine, Glycine, Proline, Serine, Tyrosine, and Selenocysteine, and/or (ii) essential amino acids selected from Phenylalanine, Valine, Tryptophan, Threonine, Isoleucine, Methionine, Histidine, Leucine, Lysine, more preferably, selected from Alanine, Cysteine, Glycine, Proline, Serine, Tyrosine, and Selenocysteine

Furthermore, in one embodiment, the medium may further optionally comprise at least one umami amino acid, as defined hereinbelow.

In one embodiment, the medium includes only one amino acid-arginine. It is to be understood that the mycelium ingredient obtainable using this medium is characterized by EUC between 30 and 100 g MSG/100 g, more preferably between 30 and 60 g MSG/100 g

Preferably, the C:N ratio ranges between 10 and 25, more preferably between 16 and 23. In one embodiment, the C:N ratio in this synthetic medium ranges between 2 and 22, preferably between 15 and 22, more preferably between 20 and 25, even more preferably between 20 and 22. In one embodiment, the C:N ratio ranges between 5 and 50. In one embodiment, the C:N ratio ranges between 1 and 5.

The present inventors have surprisingly found that by culturing the mycelium using a medium as disclosed herein, wherein the medium is devoid of any umami amino acids, as described herein, an edible mycelium ingredient may be obtained that is characterized by EUC of less than 200 g MSG/100 g, preferably characterized by EUC of less than 100 g MSG/100 g, even more preferably characterized by EUC of less than 50 g MSG/100 g. Optionally, at least one umami amino acid is used in low concentrations preferably up to 1 g/L, more preferably up to 0.2 g/L, even more preferably up to 0.1 g/L to obtain a desired EUC of mycelium less than 200 g MSG/100 g, preferably between 30 and 200 g MSG/100 g required for a specific food application.

The present inventors have surprisingly found that by culturing the mycelium using the defined medium at the fermentation conditions of this invention, an edible mycelium ingredient may be obtained that is preferably characterized by EUC ranging between 200 and 500 g MSG/100 g, preferably characterized by EUC ranging between 250 and 450 g MSG/100 g.

After one of the nutrients becomes limiting (e.g., carbon source, nitrogen source, oxygen) and fully depleted, the flasks are used to inoculate either another flask with 10 to 100 times higher volume or a pre-fermenter of similar size. Subsequently, the fermentation seed train consists of several pre-fermenters used to inoculate one after the other in volume per volume ratios between 1 and 20%. The pre-fermenters are run at 10 to 50° C. with a pH of 4 to 6 until the nutrient source is fully depleted. Depending on the inoculum size and activity, this process will take between 12 and 240 hours.

Main fermentation is finally performed at a scale of up to 400 m³ under controlled conditions (10 to 50° C., pH 3 to 8, controlled dissolved oxygen, controlled aeration rates and stirrer speed) using the media described above. Fermentation in a non-continuous mode continues until the carbon source is depleted and depending on the inoculum size and activity, this process will take between 12 and 240 hours. Ine one embodiment, the preferred pH of the fermenter related to this invention ranges between 3 and 8, more preferably between 4 and 6, even more preferably between 4 and 5. Ine one embodiment, the preferred pH of the fermenter related to this invention is preferably about 4, 4.2, 4.3, 4.5, 4.7 4.8, or 5. Preferably, the pH is controlled by acid/base titrants. In an alternative embodiment, the pH is maintained via a buffer solution, preferably selected from phosphate buffer or citrate buffer. The pH along with the C/N ratio are both a critical factor to obtain the desired growth and hence the listed properties in Table 3 (protein content, protein composition, Fiber content, and hence the effect on EUC). The pH can heavily affect protein composition and protein content as observed in literature (Calsamiglia S, Ferret A, Devant M. Effects of pH and pH fluctuations on microbial fermentation and nutrient flow from a dual-flow continuous culture system. J Dairy Sci. 2002 March; 85(3):574-9. doi: 10.3168/jds.S0022-0302(02)74111-8. PMID: 11949862.) The C/N ratio is dictated by the carbon sources and nitrogen sources present in the medium, which is varies in medium A, B, and C. This composition coupled with the fermentation conditions of pH is understood to lead different osmotic stress in fungal strains leading to different pools of total/free amino acids, hence different protein contents and compositions, leading to a different EUC. The trend observed for protein and fiber content observed for the Pleurotus species exemplified in this invention shows a surprising trend different to what is reported for the same species and another Pleurotus species, both grown on the same medium where the pH in both publication is not reported reaching a yield of at most 30% (reference 1: Food Chemistry 85(2004) 101-105; reference 2: Carbohydrate Polymers 87 (2012) 368-376, both using the medium detailed in reference 1).

The defined medium contains minerals, vitamins, and trace elements, preferably chosen from the compounds listed above and is sterilized prior to addition to the fermenter by either heat sterilisation (>121° C. for at least 20 minutes) or microfiltration, known to a person skilled in the art. At the end of the main fermentation period, the biomass is harvested by simple separation of the biomass from the cultivation supernatant using a liquid-solid separation method such as centrifugation, filtration, or sieving.

In one embodiment for all mediums of this invention, the separated biomass is washed with acidic water, preferably with a pH between 3 and 7, more preferably between 3 and 6. It is understood that this washing step treats the biomass and substantially removes any bitter compounds formed or inhibit its formation, hence influencing the EUC concentration. The acidity is preferably adjusted with citric acid, sulfuric acid, phosphoric acid or hydrochloric acid.

It is understood that the biomass yield is calculated according to the ratio of the amount biomass produced to the amount of carbon substrate consumed.

It is to be understood that according to the methods of the present invention for mediums corresponding to (usable in the preparation of) ingredient A (defined medium with one nitrogen complex source), the biomass yield is preferably up to 65%, preferably up to 60% (based on conversion of carbon source available in the medium). In one embodiment, the biomass yield of this medium corresponding to ingredient A is preferably at least 45%, more preferably at least 50%.

It is to be understood that according to the methods of the present invention for mediums corresponding to (usable in the preparation of) ingredient B (synthetic medium comprising of at least one amino acid that is essential for mycelium growth), the biomass yield is preferably up to 55%, preferably up to 50% (based on conversion of carbon source available in the medium). In one embodiment, the biomass yield of this medium corresponding to ingredient B is preferably at least 40%, more preferably at least 45%.

It is to be understood that according to the methods of the present invention for mediums corresponding to (usable in the preparation of) ingredient C (natural complex medium comprising a complex carbon source from a sidestream extract) yield up to 100%, preferably up to 99%, more preferably up to 85 to 95% biomass yield. It is understood that the biomass yield is calculated according to the ratio of the amount biomass produced to the amount of substrate(s) (e.g., C5 sugars) consumed.

The fungal strain may be grown on a natural complex medium, i.e., an extract prepared using a side stream, or an optionally preprocessed side stream. It is particularly preferred that said side stream is brewer's spent grain. The carbon to nitrogen ratio (medium) in this complex medium ranges between 5 and 50, preferably between 10 and 40, most preferably between 10 and 25. In one embodiment, the C:N ratio in this natural complex medium ranges between 2 and 18, preferably 2 and 16, more preferably between 2 and 14.

In one embodiment, the C:N ratio in this natural complex medium ranges between 2 and 18, preferably 6 and 16, more preferably between 6 and 14. In one embodiment, the C:N ratio in this natural complex medium ranges between 2 and 13, most preferably between 4 and 8. In one embodiment, the C:N ratio of this natural complex medium is about 2, about 3, about 4, about 6.5, about 7, about 8, about 9, about 10, about 11, about 13, about 13.5, or about 14 or about 20 or about 22 or about 25, depending on the C:N ratio of the sidestream extract used as the carbon source in the natural complex medium.

In one embodiment, the carbon to nitrogen ratio of the sidestream extract used as the carbon source of the natural complex medium, i.e., C:N ratio in the sidestream extract preferably ranges between 1 and 60, more preferably between 5 and 25, most probably between 10 and 20, even more preferably between 10 and 18. This C:N ratio of the sidestream extract hence influences the C:N ratio of the final natural complex medium. In one preferred embodiment, the carbon to nitrogen ratio of the sidestream extract from spent grain used as the C5-sugar carbon source of the natural complex medium preferably ranges between 1 and 50, more preferably between 1 and 25, most preferably between 5 and 25, even more preferably between 5 and 18.

Side stream can be pre-processed by using thermal or enzymatic treatment, for example. As the pre-processing step is optional, it is to be understood that the side stream can also be used as it is, without any further pre-processing.

Preferably, the protein content of the extract ranges between 1 and 200 g/l, 1 and 150 g/l, 1 and 100 g/l, 1 and 90 g/l, 1 and 80 g/l, 1 and 70 g/l, 1 and 60 g/l, 1 and 50 g/l, 1 and 40 g/l, 1 and 30 g/l, 1 and 25 g/l, 1 and 20 g/l, 1 and 15 g/l, 1 and 10 g/l, or 1 and 5 g/l. In a preferred embodiment, the protein content of the at least one extract for the complex medium comprises between 1 and 200 g/l protein, preferably between 1 and 100 g/l, more preferably between 1 and about 50 g/l, most preferably between about 5 and 45 g/l, more preferably between 7 and 30 g/l. Such protein content is essential for obtaining high EUC in the resulting biomass. In a further preferred embodiment, the protein content of the at least one extract for the complex medium comprises a protein content between 10 and 30 g/l, more preferably between 10 and 25 g/l Preferably, the protein content of the at least one extract for the complex medium comprises a protein content of about 10 g/l, more preferably about 15 g/l, even more preferably about 20 g/l.

According to the present inventors, increase in EUC is observed at a high protein concentration in the extract used in the fungal culture, i.e. 7 g/L to 30 g/L.

Preferably, the content of glutamic acid of the sidestream extract used in the medium is at least 400 mg/L, more preferably at least 500 mg/L. More preferably, the content of glutamic acid of the sidestream extract used in the medium ranges preferably 400 and 4500 mg/L, more preferably 400 and 2250 mg/L, even more preferably between 500 and 2000 mg/L. Preferably, the content of glutamic acid of the sidestream extract is preferably at least 150 mg/L, more preferably at least 200 mg/L. More preferably, the content of aspartic acid of the sidestream extract ranges preferably 150 and 1500 mg/L, more preferably between 150 and 750 mg/L, even more preferably between 220 and 650 mg/L.

The range of high glutamate concentrations achieved in the extract, which directly influences EUC, depends on residence time of the extraction in the extraction method used described below related to steam pretreatment or liquid extraction of the lignocellulosic material preferably with the addition of acids. In one embodiment, the residence time of the extraction ranges between 1 and 25 minutes, preferably between 5 and 15 minutes to reach a surprisingly higher glutamic acid content than the aspartic acid content in the extract obtainable in said extraction methods in acidic conditions. Accordingly, in this embodiment, the spent grain C5-sugar extract is obtainable in the extraction of between 1 and 25 minutes, preferably between 5 and 15 minutes under acidic conditions. In another embodiment, the residence time of the extraction ranges between 1 and 25 minutes, preferably between 1 and 20 minutes to reach a surprisingly higher glutamic acid content than the aspartic acid content in the extract obtainable in said extraction methods with a base. Accordingly, in this embodiment, the spent grain C5-sugar extract is obtainable in the extraction of between 1 and 25 minutes, preferably between 1 and 20 minutes under acidic conditions. An observed trend teaches that a higher residence time may lead to a higher glutamic acid content leading to a higher the EUC. Preferably, acidic conditions refer herein to e.g. at least 0.1 wt. % of H₂SO₄, not more than 1.6 wt. % of H₂SO₄, not more than 1.0% wt. % of H₂SO₄, preferably about 0.9 wt. % of H₂SO₄.

The fermentation medium may also be spiked or treated via substance crystallization or precipitation or via a combination of sidestreams to reach the desired C:N ratio (medium) in case of protein absence in the extract.

As preferably to be understood herein, the term about, when referring to a numeric value, refers to said value±10% of said value, more preferably it refers to said value±5% of said value, even more preferably it refers to said value±1% of said value, even more preferably it refers to said value.

The side stream as understood herein may also preferably comprise a lignocellulosic material, in particular a lignocellulosic material originating from industrial and/or agricultural side stream. Lignocellulosic material is preferably herein defined as a material that comprises dry plant matter. Preferably, said lignocellulosic material comprises cellulose, hemicellulose and lignin. Preferably, the at least one lignocellulosic material is at least one industrial and/or agricultural side stream, as defined herein. Further preferably, said lignocellulosic material is preferably solid or processed to be a powder before usage. As understood herein, the lignocellulosic material is preferably characterized by a particular colour, density, and/or mesh size distribution.

Examples of the lignocellulosic material are spent beer grain, spent grain, cereal brans, bagasse, cotton and oil press cakes from sunflower, hazelnut, shells and husks from nuts, grass and leaves waste, wood chips, coffee grounds, coffee husks, coffee silverskin, rapeseed and byproducts from the soy industry like soybean pulp (“okara”), banana leaves, banana peels, chicory roots, cassava peels, citrus pulp, cocoa, cocoa bean shell, cocoa mucilage, cocoa pod husks, coconut fibers, coconut husk, coconut shell, coffee pulp, corn cob, corn stover, cotton, cottonseed meal, cotton seeds, hemp, spent hop, pea by-products, peanut hulls, peanut meal, peanut, potato peel raw, potato tuber, eucalyptus bark, Lantana weed, switch grass, rice bran, rice husk, rice straw, spent sugar beet, sugar beet pulp, sawdust, sugarcane bagasse wet, walnut shells, wheat bran, wheat distillers grains, wheat germ, wheat straw, lupin seeds, chickpea bran, chickpea pod husks, chickpea straw, olive waste, grape marc, pear pulp, sorghum bran, sorghum germ, sorghum stalk, sorghum straw, sunflower waste, and/or tea waste. In addition, the peels or waste or pulp or pomaces of the following sidestreams are also preferably included as lignocellulosic material as encompassed by the present invention: oat, pine tree, dates, apple, apricot, spent barley, broccoli, cabbage, carrot, turnips, eggplant, kiwi, melon, alfaalfa, pineapple, pomegranate, plum, watermelon, zucchini, asparagus, beetroot, cauliflower, garlic, onion, pumpkin, squash and/or tomato.

The sidestream as referred to herein may also be understood as a sidestream from the agrifood industry. Examples of non-lignocellulosic materials, e.g., of proteinaceous materials are preferably palm oil, sugarcane scum, molasses, whey, whey permeate, wool and silk.

It is apparent to the skilled person that all the side streams as recited herein in addition to lignocellulosic material and/or proteinaceous material include sugars, minerals, and/or vitamins.

Particularly preferred side stream is brewers spent grain.

The usage of different particle sizes of spent grains leads to different results and is dictated by the application. In certain embodiments, the most abundant particle size in the particle size distribution of the spent grain ranges between 8-10 mm, preferably between 6-10 mm, more preferably between 4-6 mmm, more preferably between 2-4 mm, more preferably between 1-2 mm, more preferably between 0.2-1 mm. In a preferred embodiment, the particle sizes present in distribution are about 0.2 mm, 0.4 mm, 0.6 mm, 0.7 mm, 1 mm, 2 mm, 4 mm, 6 mm, and 10 mm. In another preferred embodiment, brewer's spent grain that is suitable for use in the method of the present invention is characterized by a particle size distribution determined by using a different set of sieves comprising a maximum between 2-4 mm up to 60 wt. %, preferably up to 50 wt. %, more preferably between 30 and 50 wt. %, most preferably between 35 and 45 wt. %, most preferably at least 35 wt. % followed by second maximum of 1-2 mm preferably up to 35 wt. %, more preferably between 15 and 30 wt. %, most preferably between 20 and 30 wt. %, followed by a third size distribution from 0.25 to 1 mm, which is preferably up to 20 wt. %, more preferably between 5 and 15 wt. %, most preferably between 8 and 15 wt. %. The sieving was performed by an air-jet sieving method, following DIN 10765 2016-07, after using an automatic sieving tower, i.e., a vibrating sieving method to get rid of particle sizes above 4 mm.

In an exemplary embodiment, the sieving residue from following vibrating sieving method is 80%, portion greater than 2.00 mm (between 2-4 mm) is 54.21 g/100 g, portion greater than 1.00 mm 33.58 g/100 g, portion greater than 0.25 mm 10 g/100 g, and the sieving residue 1.94 g/100 g.

The preparation for the fermentation of the fungal ingredients may comprise further pre-steps of pre-processing of the side stream. Herein, the following steps are explained using the example of brewer's spent grain. Preferably, pre-processing of said brewer's spent grain comprises altering the particle size or other mechanical properties of the spent grain. For example, pre-processing of spent grain may comprise grinding of the spent grain. This step is conventional and known to the skilled person. It can further undergo pre-treatment, e.g., to increase its accessible surface area by e.g., breaking down particles, e.g., by grinding, crushing, pulverization etc. Such a step, which is herein disclosed as an optional step, is known to the skilled person. In addition, and as partly discussed in the literature, (Ozturk et al., J. Inst. Brew. 108(1):23-27, 2002), the brewer's spent grain may be upon grinding sifted through a series of sieves having apertures of 850, 425 and 212 μm. Depending on the fraction, the Brewer's spent grain preparations may be considered coarse (425-850 μm), medium (212-425 μm) and fine (<212 μm), however it could be also grinded or modified to have a larger particle size, such as comprising a maximum between 0.3 to 1 mm, preferably a particle size referred to a coarse particle size by the skilled person, preferably with a maximum between 0.4 to 0.8 mm, depending on the application used.

Brewer's spent grain (BSG) or spent grain is preferably understood as a leftover or by-product of brewing industry. Preferably, spent grain is a material that remains after the mashing step and has a dry matter content of preferably between 10% and 30%. However, the dry matter content as recited herein is not meant to be limiting, as the skilled person is aware that dry matter content can be increased in pre-processing, for example by pressing, by drying or by other methods that are known to skilled person. Furthermore, the spent grain originating from other industries (for example spent grain obtainable as a by-product of production of foodstuffs) can also be used within the scope of the present invention. The skilled person shall be in a position to extend the method by including further sidestreams from the above list of lignocellulosic materials.

Preferably, the BSG comprises between 20% and 25% w/w cellulose (preferably 22%), between 23 and 28% w/w hemicellulose (preferably 25.8%), and/or between 20% and 30% w/w protein (preferably 25% w/w protein). These numbers are understood to refer to the contents of the BSG with regard to its dry mass. The BSG is characterized by a final moisture content of between 50 wt. % and 75 wt. %. Accordingly, said final moisture content may be achieved by dewatering BSG by pressing the material to a final moisture content between 50 and 75 wt. %, using the methods known to the skilled person. This however is not meant to be construed as limiting as it is further apparent to the skilled person that other means and method for dewatering said BSG can also be applied here

The extraction used for this invention to produce the mycelium ingredients is to extract C5-sugars from the lignocellulosic material comprised in BSG via a steam pretreatment, followed by a washing step with liquid water at a temperature of not more than 50° C., and to combine the so obtained extract with at least one non-carbohydrate nutrient for fungal cultivation.

Preferably, during the steam pretreatment step the lignocellulosic material comprised in BSG is contacted with steam at a temperature of between 130° C. and 180° C., preferably at a temperature of between 160° C. and 180° C., more preferably at a temperature of between 165° C. and 175° C. Preferably, during the steam pretreatment in step the lignocellulosic material comprised in BSG is contacted with steam for a time up of to 30 minutes, preferably for a time of up to 15 minutes. Preferably, during the steam pretreatment in step the lignocellulosic material comprised in BSG is contacted with steam at a temperature of between 165° C. and 175° C. and/or for a time of up to 15 minutes, preferably for a time of up to 12.5 minutes, more preferably for a time of up to 10 minutes, even more preferably for a time of up to 7.5 minutes, even more preferably for a time of up to 5 minutes, even more preferably for a time of up to 2.5 minutes, even more preferably for a time of up to 2 minutes, even more preferably for a time of up to 1 minute. More preferably, during the steam pretreatment step the lignocellulosic material comprised in BSG is contacted with steam at a temperature of between 165° C. and 175° C. and for a time of up to 15 minutes. More preferably, during the steam pretreatment in step the lignocellulosic material comprised in BSG is contacted with steam at a temperature of between 165° C. and 175° C. and for a time of up to 7.5 minutes. More preferably, during the steam pretreatment in step the lignocellulosic material comprised in BSG is contacted with steam at a temperature of between 165° C. and 175° C. and for a time of up to 2 minutes. Preferably, the temperature of between 165° C. and 175° C. relates to a temperature of about 175° C., more preferably the temperature of between 165° C. and 175° C. relates to a temperature of 170° C. Preferably the time of the steam pretreatment is at most 5 minutes, preferably at most about 4 minutes, more preferably at most about 3 minutes.

The water used for prehydrolysis with steam may comprise diluted acid, for example not more than 1% w/w of said acid. Particularly suitable are preparations of H₂SO₄ at 0.2% w/w, or 0.4% w/w. In one particular embodiment, the concentration of acid (preferably H₂SO₄) is between 1.1 and 1.6% w/w. This is in particular applicable to an embodiment wherein the lignocellulosic material is brewer's spent grain. In one embodiment of the present invention, the water used for prehydrolysis with steam may comprise diluted base, for example not more than 1% w/w of said base. Particular suitable is NaOH at 0.2% w/w. Alternatively, the water used for prehydrolysis with steam can be replaced with a phosphate buffer of pH 5.5.

The steam pretreatment is followed by a washing step with liquid water at a temperature of not more than 50° C., preferably not more than 40° C., even more preferably of not more than 30° C., even more preferably not more than 25° C. Preferably the liquid water used for washing step is at room temperature, i.e. between 20° C. and 25° C., preferably at a temperature of about 22° C., more preferably at a temperature of 22° C.

Accordingly, the aqueous extract(s) from BSG used to grow the mycelium ingredients can be further supplemented with nitrogen source(s), carbon source(s), trace element(s), vitamin(s) and/or protein composition(s). The nitrogen sources as defined herein are preferably selected from ammonia, urea, yeast extract, malt extract, corn steep liquor and peptone. More preferably, the nitrogen source(s) are ammonia and/or urea. The carbon source(s) are preferably selected from glucose, fructose, sucrose, lactose, maltose, xylose, galactose, dextrose, glycerol, and molasses, more preferably the carbon source is glucose. Preferably, no carbon sources beyond those originating from BSG are added to the medium of the present invention. The trace element(s) as defined herein may include for example iron(III) salts, copper(II) salts, zinc salts, manganese(II) salts, molybdenum salts and/or cobalt(II) salts. Vitamins as defined herein preferably include vitamins that are beneficial for the growth of fungi on the medium obtainable according to the method of the present invention, for example folic acid, riboflavin, pantothenic acid or biotin. Protein composition may be further used to supplement the aqueous extract.

In another embodiment, the fungal ingredients may be produced using a different method: extracting C5 sugars from lignocellulosic material comprised in BSG via a liquid extraction treatment with water at temperature of between 145° C. and 155° C. and/or for a time up to 70 minutes, preferably for a time up to 50 minutes, preferably at the pressure of 30 to 50 bar, and combining the so obtained extract with the at least one non-carbohydrate nutrient for fungal cultivation discussed above.

Said extraction is performed at a temperature of between 140° C. and 180° C., preferably at a temperature of between 145° C. and 175° C., more preferably at a temperature of between 145° C. and 170° C., even more preferably at a temperature of between 145° C. and 160° C., even more preferably at a temperature of 145° C. and 155° C., even more preferably at a temperature of about 150° C., and/or said extraction is performed for a time up to 70 minutes, preferably for a time up to 50 minutes. Furthermore, said extraction is preferably performed at the pressure of 30 to 50 bar.

In one embodiment of the present invention, the water used for liquid extraction treatment with water may comprise diluted acid, for example not more than 1% w/w of said acid. Particularly suitable are preparations of H₂SO₄ at 0.2% w/w, or 0.4% w/w. In one particular embodiment, the concentration of acid (preferably H₂SO₄) is between 1.1 and 1.6% w/w. This is in particular applicable to an embodiment wherein the lignocellulosic material is brewer's spent grain. In one embodiment of the present invention, the water used for liquid extraction treatment with water may comprise diluted base, for example not more than 1% w/w of said base. Particularly suitable is NaOH at 0.2% w/w. Alternatively, the water used here can be replaced with a phosphate buffer of pH 5.5.

Preferably, step of aqueous extraction of a lignocellulosic material, preferably industrial and/or agricultural side stream according to the present invention is performed with water at a pH of between 2.0 and 12.0, preferably 3.0 and 10.0, more preferably 4.0 and 8.0, even more preferably 5.0 and 8.0. The pH values as understood herein are measured under a pressure of 1.0 bar and temperature of 25° C., even though the extraction itself is performed under different conditions, as disclosed herein. Preferably, the pH is adjusted before the water is placed in contact with the at least one lignocellulosic material, preferably industrial and/or agricultural side stream. It is further understood herein that addition of acid or base to water as described herein to a final concentration of more than 1% w/w is preferably to be avoided.

C5-sugars, as defined herein, preferably refer to a fraction wherein at least 80% w/w of entire sugar content constitute pentoses (saccharides including polysaccharides comprised of sugar subunits of five carbon atom). It is noted that C5-sugars, defined herein as a fraction comprising sugars, may contain other sugars, in particular C6 sugars (sugars having 6 carbon atoms, also referred to as hexoses), as monomers and/or comprised within polysaccharides and/or oligosaccharides. Accordingly, other sugars beyond pentoses may also be extracted.

Accordingly, as referred to herein, spent grain C5-sugar extract may refer to a composition wherein at least 80 wt. % of entire sugar content constitute pentoses, more preferably at least 80% wt. % of compounds containing carbon (carbon sources) constitute pentoses.

In a further embodiment, the present invention relates to use of the fungal biomass of the present invention in production of a fungal-based food product. Accordingly, the present invention also relates to a fungal-based food product, obtainable as described herein. The fungal-based food product of the present invention may be prepared in any form known to the skilled person. For example, the fungal-based food product of the present invention may take the form of a ball (i.e., a meatball replacement), dumpling, vegetarian sausage, meat-replacement steak, meat-replacement ground meat product, meat-replacement product for preparing sandwiches, etc.

The food product according to the present invention may for example be a nutritional supplement. The nutritional supplement could be in the form of a liquid or a solid, such as a pill, lozenge, or tablet. For example, the nutritional supplement of the present invention may be a protein supplement and/or a carbohydrate supplement and/or functional drinks and beverages.

The food product as understood herein may be a dairy product, for example cheese, yoghurt, drinkable yoghurt, milk drinks and ice cream. The food product as understood herein may also relate to different embodiments of seafood products, for example a crabcake, fishcake, tuna, salmon, or shrimp, as well as to different desserts, confectionery, or bakery goods, including chocolate, brownies, or cookies, flour, starch, bread, eggs, pasta.

The food product may be texturized food product or a textured food product. Accordingly, the food product of the present invention comprises all amino acids necessary for human daily intake that cannot be synthetized in novo. Furthermore, the textured food product of the present invention is preferably heat-resistant, boiling resistant and suitable for cooking. For example, the fungal-based food product of the present invention, as described herein, may be a meat replacement product. It is noted that preferably the meat replacement product is a texturized food product or textured food product. It is further noted that the structure of the textured food product improves the acceptability of the textured food product by consumers. It is further noted that intrinsic fibrous texture of the fungal biomass of the present invention may be beneficial for producing a textured food product or a texturized food product without using conventional texturizing methods such as extrusion.

The fungal-based food product of the present invention may be further processed and or supplemented. For example, the fungal based food product of the present invention may be further supplemented with water, salt, oil and/or spices, according to protocols known to the skilled person. Further processing may also include heat and high-pressure treatment (in particular useful for a high-pressure pasteurization), brewing, boiling, baking, frying, fermenting, and/or drying of the food product. As is known to the skilled person, preservatives may be added to lengthen the shelf life of the food product of the present invention.

Preferably, food products of the present invention can be further supplemented with compositional ingredients defined as content of substances/compounds that can be described as compositional ingredients with the fungal ingredient(s) of this invention to form a fungal product.

“Compositional ingredients” is preferably understood herein as supplemented preservatives, antioxidants and acidity regulators, thickeners, stabilizers and emulsifiers, pH regulators and anti-caking agents, flavor enhancers, improving agents, stabilizers, thickening agents, colours, glazing agents and sweeteners, additives, aromatic compounds, and/or nutrients.

Preferably, preservatives include calcium carbonate, acetic acid, potassium acetate, sodium acetate, calcium acetate, lactic acid, sorbates, and malic acid.

Preferably, antioxidants and acidity regulators include ascorbic acid, sodium ascorbate, calcium ascorbate, fatty acid esters of ascorbic acid, tocopherol-rich extract, alpha-tocopherol, gamma-tocopherol, delta-tocopherol, lecithins, sodium lactate, potassium lactate, calcium lactate, citric acid, sodium citrates, potassium citrates, calcium citrates, tartaric acid (L (+)), sodium tartrates, potassium tartrate, sodium potassium tartrate, sodium malate, potassium malate, calcium malates, calcium tartrate, and triammonium citrate.

Preferably, thickeners, stabilizers and emulsifiers (or hydrocolloids) include alginic acid, sodium alginate, potassium alginate, ammonium alginate, calcium alginate, agar, carrageenan, processed euchema seaweed, locust bean gum, guar gum, tragacanth, gum arabic (acacia gum), xanthan gum, tara gum, gellan gum, sorbitol, mannitol, glycerol, konjac, pectins, cellulose, methyl cellulose, ethyl cellulose, hydroxypropyl cellulose, hydroxypropyl methyl cellulose, ethyl methyl cellulose, sodium carboxy methyl cellulose, cellulose gum, enzymatically hydrolysed carboxy methyl cellulose, sodium-potassium and calcium salts of fatty acids, magnesium salts of fatty acids, mono- and diglycerides of fatty acids, acetic acid esters of mono- and diglycerides of fatty acids, lactic acid esters of mono- and diglycerides of fatty acids, citric acid esters of mono- and diglycerides of fatty acids, tartaric acid esters of mono- and diglycerides of fatty acids, microcrystalline cellulose-Cellulose Gel, mono and diacetyl tartaric acid esters of mono- and diglycerides of fatty acids, mixed acetic and tartaric acid esters of mono- and diglycerides of fatty acids, sorbitol and mannitol.

Preferably, pH regulators and anti-caking agents include sodium carbonates, potassium carbonate, ammonium carbonates, magnesium carbonates, hydrochloric acid, potassium chloride, calcium chloride, magnesium chloride, sulphuric acid, sodium sulphates, potassium sulphates, calcium sulphate, sodium hydroxide, potassium hydroxide, calcium hydroxide, ammonium hydroxide, magnesium hydroxide, calcium oxide, magnesium oxide, fatty acids, gluconic acid, glucono delta-lactone, sodium gluconate, potassium gluconate, and calcium gluconate.

Preferably, flavor enhancers include glutamic acid, monosodium glutamate, monopotassium glutamate, calcium diglutamate, monoammonium glutamate, magnesium diglutamate, guanylic acid, disodium guanylate, dipotassium guanylate, calcium guanylate, inosinic acid, disodium inosinate, dipotassium inosinate, calcium inosinate, calcium 5′-ribonucleotides, disodium 5′-ribonucleotides, and glycine and its sodium salt.

Preferably, improving agents include L-cysteine.

Preferably, stabilizers include invertase and polydextrose.

Preferably, thickening agents include psylium husk, polydextrose, oxidized starch, monostarch phosphate, distarch phosphate, phosphate distarch phosphate, acetylated distarch phosphate, acetylated starch. Preferably, thickening agents include acetylated distarch adipate, hydroxy propyl starch, hydroxy propyl distarch phosphate, starch sodium octenyl succinate, starch-based ingredients, and acetylated oxidized starch. More preferably thickening agents include psylium husk and/or starch-based ingredients.

Preferably, colours include riboflavins, chlorophylls and chlorophyllins, anthocyanin, betanin, lycopene, copper complexes of chlorophylls and chlorophyllins, terpene compounds such as carotene compounds and xanthophyll compounds, plain caramel, caustic sulphite caramel, ammonia caramel, sulphite ammonia caramel, vegetable carbon, calcium carbonate, iron oxides and hydroxides, curcumin, tartrazine, cellulose gel, cochineal, carminic acid, carmines, azorubine, carmoisine, lutein, a cocoa powder (melanoidin), a beet powder, a tomato extract, a duckweed powder, a spirulina powder, a paprika powder (capsanthin and/or capsorubin), a turmeric powder, a blueberry powder, a strawberry powder, a berry-based colours powder, a heme powder, a lycopene powder, a betanin powder, an alfalfa powder, a saffron powder, a mint powder and an annatto extract.

Preferably, glazing agents and sweeteners include isomalt, maltitols, acesulfame potassium, aspartame, cyclamate, saccharin, sucralose, alitame, steviol glycosides, neotame, lactitol, xylitol, and erythritol.

Preferably, further additives, defined to be understood under a compositional ingredient, are selected from vitamin B12, vitamin B6, vitamin B2, vitamin B3 (also referred to as niacin), riboflavin, thiamine, vitamin A, vitamin E, omega-3 fatty acids, vitamin D2, folic acid, iodized salt (NaCl, further comprising iodine salts in an amount of up to 5% w/w), enzymes (e.g. transglutaminase, amylase), minerals (e.g. salts comprising calcium, iron, and/or potassium, etc.), flavors or flavor components (salt, pepper, garlic, onions, mushroom fruiting body pieces, vegetable pieces, ginger, turmeric, curry, sugars (i.e., sucrose, glucose, mono- or disaccharide), oils, lemon juice, orange juice, herbs and spices, yeast flakes), texturized vegetable proteins, and natural aromatic compounds. As defined herein, herbs and spices include natural aromatic compounds, such as methyl acetate, linalool, limonene, vanillin, etc. or synthetic ones like aprifloren, cinnamyl propionate, cyclohexadecanolide, and ethyl levulinate. Such further additives may improve optical visibility, flavour, nutrients, and provide additional texture.

Preferably nutrients are selected from protein rich ingredients (e.g., pea protein isolate, chickpea protein isolate, wheat gluten, egg white powder, and/or mungbean protein isolate), carbohydrate/dietary fiber rich ingredients (e.g., grain-based flours, grain-based starches, legume-based starches, fruit-based fibers, polysaccharides, starch-based ingredients, psylium husk, inulin, wheat starch, corn starch), vitamins/mineral-rich ingredients, and/or lipid rich ingredients (e.g., all types of edible oils and butters). Fiber-rich ingredients are preferably used to improve freeze thaw stability and/or juiciness.

Note that the list of compositional ingredients ends here, and it is known to a skilled person in the art that a component or substance can be classified in more than one category.

The organoleptic properties preferably may also relate to the smell attributes of being pungent, savory, floral, sour, aged, musty, earthy, off-smell; the texture attributes; the mouthfeel attributes related to juiciness and crumbliness; the taste attributes of being sweet, sour, salty, bitter, umami, metallic, astringent; the Aroma attributes preferably related to aroma complexity, aroma intensity, aroma roundness and off-flavor.

Texture attributes are defined herein as in the density, cutting strength, shear strength/force, hardness, springiness (i.e., elasticity), cohesiveness, gumminess, chewiness, adhesiveness, firmness, spreadability, stickiness, puncture force, water release, water holding capacity of the mycelial ingredient or the fungal-derived product, in particular a food product.

A cutting strength, as referred to herein, preferably describes the resistance of the food product against the intrusion of the cutting tool. Preferably, it is expressed in N.

A shear force, as referred to herein, preferably describes the ability of the biomass or the product (in particular food product) to resist unaligned forces applied to said biomass or said food product at their different parts and acting in different directions for the same weight. Preferably the forces are applied only from the top. The applied forces are collinear, also known as compression forces. Preferably, it is expressed in N.

A hardness, as referred to herein, preferably describes the force required to deform the product to a given distance. Preferably, it is expressed in N.

A springiness (i.e., elasticity), as referred to herein, preferably describes the springiness of a product, wherein the more said product is destroyed, the less springiness it will exhibit i.e., to spring back to its structural integrity after undergoing stress. Preferably, it is expressed in %, which reflects the extent of acceptable deformation with respect to the original size.

The cohesiveness of a product, as referred to herein, preferably describes the ability of a product whose structural integrity withstands compressive or tensile stress. A product is cohesive when it adheres to itself under such stress. Preferably, it is expressed in %, which reflects the extent of acceptable deformation with respect to the original size.

Gumminess, as referred to herein, preferably describes the energy required to disintegrate the food product to a state ready for swallowing. The gumminess expressed in N is calculated as the product (i.e., multiplication) of hardness N with cohesiveness %.

Chewiness, as referred to herein, is preferably described as the energy required to chew solid food. The chewiness expressed in N is the result of multiplying the gumminess N and springiness %.

Adhesiveness, as referred to herein, describes the level of stickiness of food products. If the product is subjected to pressure deformation and if the surface of the sample is sticky, a negative force will be generated, which is calculated as the negative area under the curve. A large negative value is preferably interpreted as a sticky mouthfeel.

Firmness, as referred to herein, preferably describes the toughness of the sample, with the force (N) at the maximum penetration depth being taken as sample firmness, expressed in N. The area under the positive curve (N·s) represented the total amount of force required to perform the shearing process, and it has been considered a good instrumental measurement of spreadability in cream cheeses and in other spreadable products. Smaller values in this area indicate easier spreadability. The force (N) of the maximum negative peak indicates sample stickiness represented in a negative value in N. The lower the value, the stickier the sample.

The puncture force from the penetration test, as referred to herein, preferably describes the energy required to penetrate the sample at a certain depth. Preferably, it is expressed as the area under the curve (N·s) of the positive area.

Water holding capacity (WHC) describes the ability of the material to retain the water during processing, while the release water (RW) describes the ability of the material to release the water during the processing.

“Edible” in the context of the present invention means that the edible product is safe feed (fish, cattle), safe for application as pet food and/or safe for human consumption. The food products of the present invention are products that are suitable to substitute a dairy product or a meat product. It can also be termed a vegetarian product, or even a vegan product. Suitability for substituting said products is determined by texture, mouthfeel, taste, nutritional content, water content, appearance, and other factors, which should be as similar to the product to be replaced as possible.

In certain embodiments, the edible mycelium ingredients have an elemental composition of a C to N ratio (mycelium) ranging between 1 and 50, preferably between 1 and 20, preferably between 1 and 19, more preferably between 1 and 18, more preferably between 1 and 17, more preferably between 1 and 16, more preferably between 1 and 15, more preferably between 1 and 14, more preferably between 1 and 13, more preferably between 1 and 12, more preferably between 1 and 11, more preferably between 1 and 10, more preferably between 1 and 9, more preferably between 1 and 8, more preferably between 1 and 7, more preferably between 1 and 6, more preferably between 1 and 5, more preferably between 1 and 4, more preferably between 1 and 3, 1 more preferably between and 2.

In a preferred embodiment, the edible mycelium ingredient A has an elemental composition of a C to N ratio (mycelium) ranging between 1 and 30, preferably between 1 and 15, more preferably between 5 and 10, most preferably between 6 and 8.

In a preferred embodiment, the edible mycelium ingredient B has an elemental composition of a C to N ratio (mycelium) ranging between 1 and 30, preferably between 1 and 15, preferably between 5 and 12, more preferably between 8 and 12, most preferably between 8 and 10.

In a preferred embodiment, the edible mycelium ingredient C has an elemental composition of a C to N ratio (mycelium) ranging between 1 and 30, preferably between 1 and 15, preferably between 1 and 8, more preferably between 2 and 6, most preferably between 4 and 6.

In a further embodiment, the present invention relates to edible fibrous mycelium mass, as described herein. Preferably, the edible fibrous mycelium mass is derived from submerged fermentation.

In another embodiment, without any extra ribonucleic acid (RNA) reduction step in the production process of the ingredients via additional heating or pH treatment, the method of this invention leads preferably to an inherent RNA level of the ingredients of 4 wt. % at most, preferably 2 wt. % at most on a dry bases for all the disclosed mycelial ingredients. This is applicable for each of the ingredients A, B, and C.

In one embodiment, the value of RNA for the edible mycelial ingredients ranges between 0.1 and 4 wt. %. preferably between 0.1 and 2 wt. %, preferably between 0.1 and 1.5 wt. %, more preferably between 0.1 and 1 wt. %, more preferably between 0.1 and 0.4 wt. % on a dry basis. In a preferred embodiment, the inherent RNA level for ingredient A ranges between 0.1 and 4 wt. %, preferably between 0.1 wt. % to 2 wt. %, more preferably between 0.4 wt. % and 1.88 wt. % on a dry basis. In a preferred embodiment, the inherent RNA level for ingredient B ranges between 0.1 and 4 wt. %, preferably between 0.1 wt. % to 2 wt. %, more preferably between 0.4 wt. % and 1.7 wt. % on a dry basis. In a preferred embodiment, the inherent RNA level for ingredient C ranges between 0.1 and 4 wt. %, preferably between 0.1 wt. % to 2 wt. %, more preferably between 0.5 wt. % and 1.9 wt. % on a dry basis.

In another embodiment, the edible mushroom ingredients contain chitin ranging between 1 and 35 wt. %, preferably between 1 and 20 wt. %, more preferably between 1 and 15 wt. %, more preferably between 1 and 14 wt. %, more preferably between 1 and 13 wt. %, more preferably between 1 and 12 wt. %, more preferably between 1 and 11 wt. %, more preferably between 1 and 10 wt. %, more preferably between 1 and 9 wt. %, more preferably between 1 and 8 wt. %, more preferably between 1 and 7 wt. %, more preferably between 1 and 6 wt. %, more preferably between 1 and 5 wt. %, more preferably between 1 and 4 wt. %, more preferably between 1 and 3 wt. %, more preferably between 1 and 2 wt. % on a dry weight basis. In a preferred embodiment, the edible mushroom ingredients contain chitin in the range of 5 to 15 wt. %, more preferably between 6 and 12 wt. %, and most preferably between 6 and 9 wt. % on a dry weight basis.

In certain embodiments, the mycelium ingredients have an Ergothioneine value ranging between 1-1000 mg/kg, preferably between 1-900 mg/kg, more preferably between 1-800 mg/kg, more preferably between 1-700 mg/kg, more preferably between 1-600 mg/kg, more preferably between 1-500 mg/kg, more preferably between 1-400 mg/kg, more preferably between 1-300 mg/kg, more preferably between 1-200 mg/kg, more preferably between 1-100 mg/kg, more preferably between 1-50 mg/kg, more preferably between 1-25 mg/kg. In a preferred embodiment, the mycelium ingredient contains an Ergothioneine value ranging 25 to 1000 mg/kg, preferably between 50 and 700 mg/kg, more preferably between 80 and 600 mg/kg. In a preferred embodiment, the mycelium ingredient A contains an Ergothioneine value ranging between 70 and 270 mg/kg, said value is preferably achieved in 5 days. In a preferred embodiment, the mycelium ingredient B contains an Ergothioneine value ranging between 100 and 350 mg/kg, said value is preferably achieved in 5 days. In a preferred embodiment, the mycelium ingredient C contains an Ergothioneine value ranging between 300 and 800 mg/kg, said value is preferably in 5 days, preferably ranging between 350 and 800 mg/kg, said value is preferably in 5 days, most preferably ranging between 400 and 800 mg/kg, said value is preferably achieved in 5 days. As it is to be understood herein, ergothioneine value preferably refers to the amount of ergothioneine expressed in mg per kg of the mycelial ingredient, expressed on a dry mass basis.

As surprisingly found by the present inventors, when using Pleurotus pulmonarius as the fungal strain in a fermentation system for 5 days, the mycelium ingredient A preferably has an

Ergothioneine content ranging between 70 and 100 mg/kg, ingredient B preferably contains a value ranging between 110 and 150 mg/kg of Ergothioneine, while for ingredient C shows the highest content for Ergothioneine with a range between 380 and 455 mg/kg, with an average value reached of 433 mg/kg, which is a higher value compared to disclosed patents in this field of Ergothioneine using Pleurotus pulmonarius to produce this functional substance.

In certain embodiments, the content of ergosterol in the mycelium ingredients ranges between 0 and 50 mg/g, preferably between 0 and 25 mg/g, more preferably between 0 and 15 mg/g, more preferably between 0 and 12.5 mg/g, more preferably between 0 and 10 mg/g, more preferably between 0 and 9 mg/g, more preferably between 0 and 8 mg/g, more preferably between 0 and 7 mg/g, more preferably between 0 and 5 mg/g, more preferably between 0 and 4 mg/g, more preferably between 0 and 3 mg/g, more preferably between 0 and 2 mg/g, and more preferably between 0 and 1 mg/g. In a preferred embodiment, the content of ergosterol in the mycelium ingredients ranges between 0 and 8 mg/g, more preferably between 1 and 6 mg/g, most preferably between 2 and 6 mg/g. In a further preferred embodiment, the mycelium ingredient A and B contain an ergosterol range between 0 and 4 mg/g, more preferably between 2 and 4 mg/g. In a further preferred embodiment, the mycelium ingredient C contains an ergosterol range between 0 and 8 mg/g, more preferably between 2 and 7 mg/g, more preferably between 4 and 7 mg/g, most preferably between 5 and 6 mg/g.

In certain embodiments, the flavors in terms of the known flavor 5′ nucleotides (5′NMP) in g/kg, which include 5′-inosine monophosphate (IMP), 5′-guanosine monophosphate (GMP) and 5′-adenosine monophosphate (AMP) are measured. In a preferred embodiment, IMP is absent with a value of 0 g/kg. In a further embodiment, the umami taste coming from 5′-nucleotides can be further intensified by at least 30%, preferably at least more preferably by at least 50%, upon an enzymatic treatment, preferably with 5′-Adenylic deaminase, to convert AMP to IMP.

In one embodiment, the edible mushroom ingredients show a good richness of 5′NMP containing 5′-guanosine monophosphate (GMP) and 5′-adenosine monophosphate (AMP) ranging between 0.1 and 40 g/kg, preferably between 0.1 and 20 g/kg preferably between 0.1 and 15 g/kg, more preferably between 0.1 and 12.5 g/kg, more preferably between 0.1 and 10 g/kg, more preferably between 0.1 and 8 g/kg, more preferably between 0.1 and 6 g/kg, more preferably between 0.1 and 5 g/kg, more preferably between 0.1 and 4 g/kg, more preferably between 0.1 and 3 g/kg, more preferably between 0.1 and 2 g/kg.

In a preferred embodiment, this value of 5′NMP for ingredient A ranges between 1 and 20 g/kg, more preferably between 2 and 6 g/kg, most preferably between 3 and 6 g/kg.

In a preferred embodiment, this value of 5′NMP for ingredient B ranges between 0.1 and 15 g/kg, more preferably between 0.1 and 5 g/kg, most preferably between 0.1 and 3 g/kg.

In a preferred embodiment, this value of 5′NMP for ingredient C ranges between 0.1 and 40 g/kg, more preferably between 5 and 20 g/kg, most preferably between 8 and 20 g/kg.

In another preferred embodiment, this value of 5′NMP for the mycelium ingredient A 40 g/kg at most, preferably 20 g/kg at most.

In another preferred embodiment, this value of 5′NMP for the mycelium ingredient B 40 g/kg at most, preferably 20 g/kg at most.

In another preferred embodiment, this value of 5′NMP for the mycelium ingredient C 40 g/kg at most, preferably 20 g/kg at most.

It is observed that the mycelia grown on the spent grain extract led to a 10-fold increase in ingredient of 5′NMP.

In another embodiment, the mycelium ingredient C shows a high concentration of Uridine monophosphate (UMP), preferably in the range of 2.5 to 5 g/kg.

In certain embodiments the mycelial ingredients preferably contain all the 20 essential amino acids. In a preferred embodiment, the mycelial ingredients comprise of the amino acids: alanine, asparagine, aspartate, arginine, tryptophan, glycine, glutamic acid, glutamine, methionine, phenylalanine, serine, valine, cystine, proline, leucine, tyrosine, threonine, isoleucine, histidine, and lysine.

In a preferred embodiment, the edible mycelium ingredients contain branched-chain amino acids (BCAAs) of at least about 10 wt. %, preferably at least about 15 wt. %, more preferably at least about 19 wt. %, more preferably at least about 20 wt. %, more preferably at least about 21 wt. %, more preferably at least about 22 wt. %, more preferably at least about 23 wt. % of the total amount of amino acids present (i.e., of the total protein, as the amino acids present as referred to herein relate to the total content of free amino acids as well as amino acids comprised in the proteins and peptides).

In a preferred embodiment, the edible mycelium ingredients contain umami amino acids of at least about 10 wt., preferably at least about 15 wt. %, more preferably at least about 19 wt. %, more preferably at least about 20 wt. %, more preferably at least about 21 wt. %, more preferably at least about 22 wt. %, more preferably at least about 23 wt. % of the total amount of amino acids present (i.e., of the total protein, as the amino acids present as referred to herein relate to the total content of free amino acids as well as amino acids comprised in the proteins and peptides).

It is herein understood that essential amino acids are amino acids that cannot be synthesized by the organism efficiently enough to supply its demand and must be supplied from the diet.

In a preferred embodiment, the edible mycelium ingredients contain essential amino acids of at least about 30 wt. %, preferably at least about 35 wt. %, more preferably at least about 40 wt. % of the total amount of amino acids present (i.e., of the total protein, as the amino acids present as referred to herein relate to the total content of free amino acids as well as amino acids comprised in the proteins and peptides).

In a preferred embodiment, the total amount of amino acids in ingredient A corresponds to 150 to 300 mg/g, preferably between 200 and 300 mg/g, more preferably between 250 and 300 mg/g, most preferably between 250 and 280 mg/g.

In a preferred embodiment, the total amount of amino acids in ingredient B, grown on a minimal medium, corresponds to 100 to 200 mg/g, preferably between 120 and 180 mg/g, more preferably between 150 and 180 mg/g.

In a preferred embodiment, the total amount of amino acids in ingredient B, grown on a spent grain extract, corresponds to 200 to 700 mg/g, preferably between 250 and 550 mg/g, more preferably between 300 and 500 mg/g, most preferably between 350 and 450 mg/g.

In another preferred embodiment, the edible mycelium ingredient A, wherein the amount of each of the BCAAs and the amount of umami amino acids separately range between 40 and 100 mg/g, preferably between 50 and 60 mg/g each respectively.

In another preferred embodiment, the edible mycelium ingredient B, wherein the amount of each of the BCAAs and the amount of umami amino acids separately range between 20 and 80 mg/g, preferably between 30 and 40 mg/g each respectively.

In another preferred embodiment, the edible mycelium ingredient C, wherein the amount of the BCAAs ranges 50 and 150 mg/g, preferably between 65 and 75 mg/g and the amount of umami amino acids ranges between 50 and 150 mg/g, preferably between 70 and 100 mg/g, most preferably between 75 and 85 mg/g.

In certain embodiments, the EUC concentration of the mycelium ingredients ranged between 1 and 25 000%. In a preferred embodiment, the EUC concentration of the mycelium ingredients range between 1 and 20 000%, preferably between 1 and 15 0000%, more preferably between 1 and 13 000%, more preferably between 1 and 12 000%, more preferably between 1 and 10 000%, more preferably between 1 and 5000%, more preferably between 1 and 2500%, more preferably between 1 and 1500%, more preferably between 1 and 1000%, more preferably between 1 and 600%.

In a preferred embodiment of mycelium ingredient A, the EUC concentration ranges between 1 and 1000%, more preferably between 250 and 800%, even more preferably between 250 and 500%. Preferably the EUC concentration for mycelium ingredient A is about 300%. In another preferred embodiment of mycelium ingredient A, the EUC is between 200 and 500 g MSG/100 g.

Preferably, said mycelium ingredient A is characterized by insoluble fiber content of between 25 and 45% w/w, more preferably between 30 and 45% w/w, e.g. 34% w/w. Herein, % w/w refer to the content of insoluble fiber in dry mass.

In a preferred embodiment for mycelium ingredient B, the EUC concentration ranges between 1 and 600%, preferably between 1 and 200%, more preferably between 10 and 100%, even more preferably between 30 and 60%. Preferably the EUC concentration for mycelium ingredient B is about 34%. Alternatively, in a preferred embodiment of mycelium ingredient B, the EUC is less than 200 g MSG/100 g, more preferably the EUC is less than 100 g MSG/100 g. Alternatively, in one embodiment of mycelium ingredient B, the EUC is preferably between 30 and 200 g MSG/100 g, even more preferably is between 30 and 60 g MSG/100 g.

Preferably, said mycelium ingredient B is characterized by insoluble fiber content of between 30 and 60% w/w, preferably of between 40 and 60% w/w, e.g. 54% w/w. Herein, % w/w refer to the content of insoluble fiber in dry mass.

In a preferred embodiment for mycelium ingredient C, the EUC concentration ranges between 1 and 10000%, more preferably between 500 and 5000%, even more preferably between 2000 and 4000%, even more preferably between 2500 and 3500%. Preferably the EUC concentration for mycelium ingredient C is about 2890%. In a preferred embodiment for mycelium ingredient C, it is characterized by an EUC concentration of at least 500%, more preferably at least 1000%, even more preferably 1500% and even more preferably at least 2000%.

Preferably, said mycelium ingredient C is characterized by insoluble fiber content of up to 35% w/w, more preferably up to 30% w/w e.g. 25% w/w. Herein, % w/w refer to the content of insoluble fiber in dry mass.

It is noted that said above EUC concentrations are achieved in 5 days.

Uronic acids, particularly D-galacturonic and D-glucuronic acid which are analyzed for in the mycelial ingredients, are highly valuable substances and they can play a role as antioxidants and detoxifying and inactivating agents of various substances in the human body. In certain embodiments the content of both uronic acids in the mycelial ingredients after acid hydrolysis, are in the range of 0-50 wt. %, preferably 0-25 wt. %, more preferably 0-15 wt. %, more preferably 0-10 wt. %, more preferably 0-5 wt. %, more preferably 0-2.5 wt. %, more preferably 0-2 wt. %. In a preferred embodiment, the content of the uronic acids in the mycelial ingredients ranges between 0.1 to 15 wt. %, more preferably between 0.1 and 5 wt. %, most preferably between 1 and 2 wt. %.

In certain embodiments, the total phenolic content of the mycelium ingredients ranges between 1 and 200 mg GAE/g, between 1 and 150 mg GAE/g, between 1 and 100 mg GAE/g, between 1 and 50 mg GAE/g, between 1 and 25 mg GAE/g, between 1 and 10 mg GAE/g, between 1 and 5 mg GAE/g. In a preferred embodiment, the total phenolic content of the mycelium ingredients ranges between 1 and 150 mg GAE/g, preferably between 1 and 15 mg GAE/g, more preferably between 1 and 5 mg GAE/g.

In certain embodiments, the total flavonoid content of the mycelium ingredients ranges between 1 and 200 mg QE/g, between 1 and 150 mg QE/g, between 1 and 100 mg QE/g, between 1 and 50 mg QE/g, between 1 and 25 mg QE/g, between 1 and 10 mg QE/g, between 1 and 5 mg QE/g. In a preferred embodiment, the total flavonoid content of the mycelium ingredients ranges between 1 and 150 mg QE/g, preferably between 1 and 15 mg QE/g, more preferably between 1 and 5 mg QE/g.

In a specific embodiment, the mycelium ingredient A has a total phenolic content ranging between 1 and 5 mg GAE/g, preferably about 3.2 mg GAE/g.

In a specific embodiment, the mycelium ingredient B has a total phenolic content ranging between 1 and 5 mg GAE/g, preferably about 2.7 mg GAE/g.

In a specific embodiment, the mycelium ingredient C has a total phenolic content ranging between 1 and 5 mg GAE/g, preferably about 4.5 mg GAE/g.

In a specific embodiment, the mycelium ingredient A has a total flavonoid content ranging between 1 and 5 mg QE/g, preferably about 1.7 mg QE/g.

In a specific embodiment, the mycelium ingredient B has a total flavonoid content ranging between 1 and 5 mg QE/g, preferably about 2.5 mg QE/g.

In a specific embodiment, the mycelium ingredient C has a total flavonoid content ranging between 1 and 5 mg QE/g, preferably about 3 mg QE/g.

It is obvious that the extracts contained lower total flavonoid contents than the total phenolic contents. The results suggest that other compounds besides flavonoids are the major phenolic substances present in the tested strain, especially for ingredient A and B. In one embodiment, the flavonoid content of the mycelium ingredient A constitutes around 35% to 60% of the total phenolic content, preferably around 45 to 55% of the total phenolic content, preferably around 55%. In one embodiment, the flavonoid content of the mycelium ingredient B constitutes around 70 to 95% of the total phenolic content, preferably around 80 to 95% of the total phenolic content, preferably around 93%. In one embodiment, the flavonoid content of the mycelium ingredient C constitutes around 50 to 80% of the total phenolic content, preferably 60 to 70% of the total phenolic content, preferably around 65%.

In one embodiment, the total polyphenols of the mycelium ingredients ranges between 1-2000 mg/kg, 1-1000 mg/kg, 1-900 mg/kg, 1-800 mg/kg, 1-700 mg/kg, 1-600 mg/kg, 1-500 mg/kg, 1-400 mg/kg, 1-300 mg/kg, 1-200 mg/kg, 1-100 mg/kg, 1-50 mg/kg, or 1-25 mg/kg. In a preferred embodiment, the total polyphenols of the mycelium ingredients range between 1 and 1500 mg/kg, preferably between 25 and 1000 mg/kg, more preferably between 50 and 900 mg/kg, said value is preferably achieved in 5 days. This is applicable for ingredients A. B, and C.

In a specific embodiment, the mycelium ingredient A has a total polyphenols ranging between 1 and 500 mg/kg, preferably between 50 and 200 mg/kg, more preferably around 165 mg/kg. In a further preferred embodiment, the mycelium ingredient A has a total polyphenols, wherein around 35 to 45 wt. % of the total polyphenols correspond to catechin, around 35 to 45 wt. % of the total polyphenols correspond to protocatechuic acid, and a rest about 1 to 10 wt. % of each of quercetin, chlorogenic acid, and/or syringic acid.

In a specific embodiment, the mycelium ingredient B has a total polyphenols ranging between 1 and 250 mg/kg, preferably 50 to 150 mg/kg, more preferably around 75 mg/kg. In a further preferred embodiment, the mycelium ingredient B has a total polyphenol, wherein around 25 to 35 wt. % of the total polyphenols correspond to catechin, around 35 to 45 wt. % of the total polyphenols correspond to protocatechuic acid, around 10 to 20 wt. % of the total polyphenols correspond to quercetin, and a rest about 5 to 15 wt. % corresponding to chlorogenic acid.

In a specific embodiment, the mycelium ingredient C has a total polyphenols ranging between 1 and 1500 mg/kg, preferably 100 and 1000 mg/kg, more preferably 250 to 950 mg/kg, more preferably around 650 mg/kg. In a further preferred embodiment, the mycelium ingredient C has a total polyphenols, wherein around 45 to 55 wt. % of the total polyphenols correspond to catechin, around 45 to 55 wt. % of the total polyphenols correspond to protocatechuic acid, around 1 to 5 wt. % of the total polyphenols correspond to syringic acid.

An ethanol extract of the mycelium ingredients was prepared with an accelerated solvent extractor using 96% ethanol as performed in Processes. 2020; 8(7):803. https://doi.org/10.3390/pr8070803 to perform an antioxidant assay and assess the antioxidant activity which was determined by the ability to scavenge stable 1,1-diphenyl-2-picrylhydrazyl (DPPH) free radical following the protocol of scavenging activity (A potential antioxidant resource: endophytic fungi from medicinal plants. Econ. Bot. 61, 14-30.). This is usually defined by an EC50 value, preferably defined as the concentration of antioxidants required for 50% scavenging of DPPH radicals in specified time periods.

In certain embodiments the EC50 for the mycelium ingredients ranges between 1 and 100 mg/ml, preferably between 1 and 50, more preferably between 1 and 15 mg/ml. In a preferred embodiment, the EC50 for the mycelium ingredient A ranges between 1 and 25 mg/ml, preferably about 10.5 mg/ml.

In a preferred embodiment, the EC50 for the mycelium ingredient B ranges between 1 and 25 mg/ml, preferably about 13 mg/ml. In a preferred embodiment, the EC50 for the mycelium ingredient C ranges between 1 and 25 mg/ml, preferably about 9 mg/ml.

In a preferred embodiment, the mycelium ingredient C has the highest DPPH radical-scavenging activity, preferably has an EC50 of about 9 mg/ml. This correlates well with the phenolic content of ingredient C having the highest phenolic content, therefore the phenolic content and antioxidant activity are correlating well.

In a preferred embodiment, the mycelium ingredient A has the second highest DPPH radical-scavenging activity, preferably has an EC50 of about 10.5 mg/ml.

In a preferred embodiment, the mycelium ingredient B has the second third DPPH radical-scavenging activity, preferably has an EC50 of about 13 mg/ml.

The following sugars glucan, xylan, arabinan, galactan, mannan, rhamnan were measured in the mycelial ingredients after acid hydrolysis. In certain embodiments, the total lignocellulosic sugars detected from the hydrolysis of the mycelial ingredients ranges between 1 and 100%, preferably 1 and 90%, more preferably 1 and 80%, more preferably 1 and 70%, more preferably 1 and 60%, more preferably 1 and 50%, more preferably 1 and 40%, more preferably 1 and 30%, more preferably 1 and 25%, more preferably 1 and 20%, more preferably 1 and 15%, more preferably 1 and 10%, more preferably 1 and 5%. In a preferred embodiment, the total lignocellulosic sugars detected from the hydrolysis of the mycelial ingredient A ranges between 20 and 60%, more preferably between 25 and 45%, wherein the most abundant sugar is glucan constituting 80 to 90% of the total measured sugars (the glucan content is preferably 25 to 40 wt. % in its absolute value).

In a further preferred embodiment, the total sugars detected from the hydrolysis of the mycelial ingredient B ranges between 30 and 80%, more preferably between 20 and 70%, most preferably between 40 and 60%, wherein the most abundant sugar is glucan constituting 85 to 95% of the total sugars (the glucan content is preferably 40 to 50 wt. % in its absolute value). In a further preferred embodiment, the total sugars detected from the hydrolysis of the mycelial ingredient C ranges between 1 and 50%, more preferably between 1 and 35%, most preferably between 1 and 30%, wherein the most abundant sugar is glucan constituting 75 to 85% of the total sugars (the glucan content is preferably 10 to 25 wt. % in its absolute value).

In another preferred embodiment, the most abundant sugar detected from the hydrolysis of the mycelial ingredients A, B or C is glucan ranging between 70 and 95% out of the total sugars.

As understood herein, the term “insoluble fiber” preferably refers to part of the dietary fiber, which does not dissolve in water. Said insoluble fiber preferably comprises chitin and β-glucan and is preferably distinguishable from the insoluble fiber of plant origin, which comprises plant cellulose and/or hemicellulose, but do not comprise chitin. It is known that insoluble fibers help the human body with processing waste better and improve bowel health and reduce risks for colorectal conditions.

Preferably, the edible fibrous mycelium ingredients have an insoluble fiber content of at least 20%, preferably at least 30%, preferably at least 40%, preferably at least 50%, preferably at least 60%. More preferably, said edible fibrous mycelium has an insoluble fiber content of between 20% w/w and 60% w/w. Even more preferably, said edible fibrous mycelium has an insoluble fiber content of between 40% w/w and 55% w/w.

In a preferred embodiment, the insoluble fiber content of mycelium ingredient A ranges between 30 and 60 wt. %, preferably between 30 and 40 wt. %.

In a preferred embodiment, the insoluble fiber content of mycelium ingredient B ranges between 40 and 60 wt. %, preferably between 40 and 50 wt. %.

In a preferred embodiment, the insoluble fiber content of mycelium ingredient C ranges between 10 and 40 wt. %, preferably between 20 and 30 wt. %.

It is preferably envisaged that the mycelium used for dairy products versus meat products versus other products may be tailored to the specific requirements of the final product in terms of fiber content, protein content, nutrients, taste, etc.

In a certain preferred embodiment, the insoluble fiber content of the edible fibrous mycelium used in a meat analogue product formulation is between 20 and 60 wt. %, preferably between 40 and 50 wt. %, preferably about 45 wt. % In another preferred embodiment, the insoluble fiber used in a meat analogue is about 35 wt. %. In another preferred embodiment, the insoluble fiber used in a meat analogue is about 25 wt. %. In another preferred embodiment, the insoluble fiber used in a meat analogue is about 45 wt. %. In one embodiment, the edible fibrous mycelium in the meat analogue has an insoluble fiber content of at least 20 wt. %, preferably of at least 30 wt. %, preferably of at least 45 wt. %., preferably of at least 50 wt. %

In a further preferred embodiment, the insoluble fiber content of the edible fibrous mycelium used in dairy analogue products or other products (other than meat analogues) is at least 20 wt. %, more preferably at least 30 wt. %, more preferably at least 40 wt. %. More preferably, said edible fibrous mycelium has an insoluble fiber content of between 40 wt. % and 60 wt. %. Even more preferably, said edible fibrous mycelium has an insoluble fiber content of between 40 wt. % and 55 wt. %. Even more preferably, said edible fibrous mycelium has an insoluble fiber content of between 40 and 50 wt. %. Most preferably, said edible fibrous mycelium has an insoluble fiber content of about 45 wt. %. Most preferably, said edible fibrous mycelium has an insoluble fiber content of at least 50 wt. %. It is to be understood that the weight as referred to herein relates to dry mass of said mycelium. In one embodiment, the edible fibrous mycelium has an insoluble fiber content of at least 40 wt. %, preferably of at least 50 wt. %, more preferably of at least 60 wt. %.

The edible fibrous mycelium of the present invention preferably has a protein content of between 10 wt. % and 65 wt. %, most preferably between 30 wt. % and 60 wt. %. In certain embodiments the protein content is about at least 30 wt. %, at least 40 wt. %, at least 60 wt. %.

In a preferred embodiment, the protein content of mycelium ingredient A ranges between 30 and 50 wt. %, preferably between 30 and 40 wt. %.

In a preferred embodiment, the protein content of mycelium ingredient B ranges between 30 and 50 wt. %, preferably between 30 and 40 wt. %.

In a preferred embodiment, the protein content of mycelium ingredient C ranges between 30 and 65 wt. %, preferably between 45 and 65 wt. %.

The protein content of the mycelium mass can be adjusted by choosing fermentation conditions, which control the protein content of the obtained mycelium mass. Such conditions are known to the skilled person and involve adjusting the contents of the fermentation medium to control the uptake and therefore the composition of the mycelium mass to be obtained. For example, the ratio between carbon and nitrogen source in the medium can be altered, such as providing nitrogen in different grades of excess. This will influence the insoluble fiber content accordingly i.e., higher protein, means lower insoluble fiber content as seen in table 3.

In one embodiment the edible mycelium ingredients, having a carbohydrate content (non-fiber carbohydrates i.e., carbohydrates not encompassing fibers) of 5% at most, preferably a content of 1% at most. In a preferred embodiment, the edible mycelium ingredients, having a carbohydrate content of 0.5% at most, most preferably less than 0.01%.

In one embodiment, the edible mycelium ingredients have a beta-glucan content of at least 80% of the total glucans.

In one embodiment, the present invention relates to the edible mycelium ingredient A, wherein the total glucans content ranges between 20 and 35 wt. %, preferably between 25 and 35 wt. %. In one embodiment, the present invention relates to the edible mycelium ingredient B, wherein the total glucans content ranges between 25 and 50 wt. %, preferably between 30 and 40 wt. %.

In one embodiment, the present invention relates to the edible mycelium ingredient C, wherein the total glucans content ranges between 10 and 35 wt. %, preferably between 10 and 20 wt. %.

In one embodiment, the present invention relates to the mycelium ingredients A. B. or C, wherein at least 96 wt. % of its polyunsaturated fatty acids content is linoleic acid (omega-6 fatty acid). In a specific preferred embodiment, the present invention relates to the mycelium ingredient A, wherein at least 98 wt. % of its polyunsaturated fatty acids content is linoleic acid (omega-6 fatty acid). In a specific preferred embodiment, the present invention relates to the mycelium ingredient B, wherein at least 97 wt. % of their polyunsaturated fatty acids content is linoleic acid (omega-6 fatty acid). In a specific preferred embodiment, the present invention relates to the mycelium ingredient C, wherein at least 96 wt. % of their polyunsaturated fatty acids content linoleic acid (omega-6 fatty acid). In a further specific embodiment, the present invention relates to the mycelium ingredient C, having an enriched omega-6 fatty acid (linoleic acid) ranging between 1 and 9 wt. %, more preferably between 2 and 5 wt. %, most preferably about 3.5 wt. %.

In one embodiment, the mycelium ingredients A, B, and C have a fat content of at most 15 wt. %, preferably at most 10 wt. %. In a specific preferred embodiment, the present invention relates to the mycelium ingredient A, wherein the fat content ranges between 0.5 and 5 wt. %, preferably between 0.1 and 3 wt. %. In a specific preferred embodiment, the present invention relates to the mycelium ingredient B, wherein the fat content ranges between 0.5 and 5 wt. %, preferably between 0.1 and 3 wt. %. In a specific preferred embodiment, the present invention relates to the mycelium ingredient C, wherein the fat content ranges between 1 and 10 wt. %, preferably between 3 and 10 wt. %, more preferably between 3 and 8 wt. %. In a specific preferred embodiment, the present invention relates to the mycelium ingredient C, wherein the fat content is at most 8 wt. %.

In one embodiment, the mycelium ingredients contain no mycotoxins. The mycotoxins were analyzed via a liquid chromatography with tandem mass spectrometry (SOP M 3650). No mycotoxins were present above their detection limit in μg/kg. The mycotoxins are selected from Aflatoxin B1, Aflatoxin B2, Aflatoxin G1, Aflatoxin G2, Ochratoxin A, Deoxynivalenol (DON), Zearalenone, 3-Acetyl-Deoxynivalenol, 15-Acetyl-Deoxynivalenol, Nivalenol, T-2 Toxin, HT-2 Toxin, 4, 15-Diacetoxyscirpenol, Fusarenon-X, Fumonisin B1, and Fumonisin B2.

In another embodiment, polyaromatic hydrocarbons were analyzed using gas chromatography-mass spectrometry (SOP M 2920). No polyaromatic hydrocarbons were detected above their detection limit in μg/kg. The polyaromatic hydrocarbons are selected from Benzo (a) anthracene, Benzo (c) fluorene, Chrysene, Cyclopenta (c,d) pyrene, 5-Methylchrysene, Benzo (b) fluoranthene, Benzo (k) fluoranthene, Benzo (j) fluoranthene, Benzo (a) pyrene, Indeno (1,2,3-cd) pyrene, Dibenzo (ah) anthracene, Benzo (ghi) perylene, Dibenzo (a,l) pyrene, Dibenzo (a,e) pyrene, Dibenzo (a,i) pyrene, Dibenzo (a,h) pyrene.

In certain embodiments, the calorific value of the mycelial ingredients ranges between 1 and 1000 Kcal/100 g, preferably between 1 and 900 Kcal/100 g, more preferably between 1 and 800 Kcal/100 g, more preferably between 1 and 700 Kcal/100 g, more preferably between 1 and 500 Kcal/100 g, more preferably between 1 and 400 Kcal/100 g, more preferably between 1 and 300 Kcal/100 g, more preferably between 1 and 200 Kcal/100 g, more preferably between 1 and 100 Kcal/100 g, more preferably between 1 and 50 Kcal/100 g, more preferably between 1 and 25 Kcal/100 g. In a preferred embodiment, the calorific value of the mycelial ingredients ranges between 1 and 800 Kcal/100 g, preferably between 200 and 800 500 Kcal/100 g, more preferably between 300 and 600 Kcal/100 g, most preferably between about 300 and 500 Kcal/100 g.

In a preferred embodiment, for pore diameters below 1 mm, for the edible mushroom ingredient A, 55 to 65%, preferably about 58% of the pore-volume corresponds to a pore diameter between 1000 and 30 μm with the most frequent pore diameter in the range of 85-185 μm. In the second range of 30 to 2 μm, corresponding to about 35 to 45%, preferably 42% of the pore-volume, the most frequent pore diameter peak is equal to 16 μm, which is also the most frequent pore diameter in the range of 1000-2 μm. The specific pore volume of ingredient A is 9 cm3/g and the median pore diameter is 44.5 μm.

In a preferred embodiment, for pore diameters below 1 mm, for the edible mushroom ingredient B, about 75 to 85%, preferably 81.5% of the pore-volume corresponds to a pore diameter between 1000 and 30 μm with the most frequent pore diameter equal to 147 μm, which is also the most frequent pore diameter in the range of 1000-2 μm. In the second range of 30 to 2 μm, corresponding to about 15 to 25%, preferably 18.5% of the pore-volume, the most frequent pore diameter peak is equal to 15 μm. The specific pore volume of ingredient B is 4.94 cm3/g and the median pore diameter is 143 μm.

In a preferred embodiment, for pore diameters below 1 mm, for the edible mushroom ingredient C, 15% to 25%, preferably about 20% of the pore-volume corresponds to a pore diameter between 1000 and 20 μm and 75 to 85%, preferably about 80% of the pore-volume corresponds to a pore diameter between 20 to 2 μm with the most frequent pore diameter peak of 5.5 μm. The specific pore volume of ingredient B is 2.46 cm3/g and the median pore diameter is 7.1 μm.

Ingredient C pore volume is much lower, and the pores are smaller compared to A and B.

In a preferred embodiment, the BET surface of ingredients A, B and C was also measured using Krypton because it is the suitable adsorbate for measuring low surface areas. The surface areas of the ingredients without further grinding are preferably: 0.79 m²/g for A, 0.67 m²/g for B, and 1.59 m²/g for C.

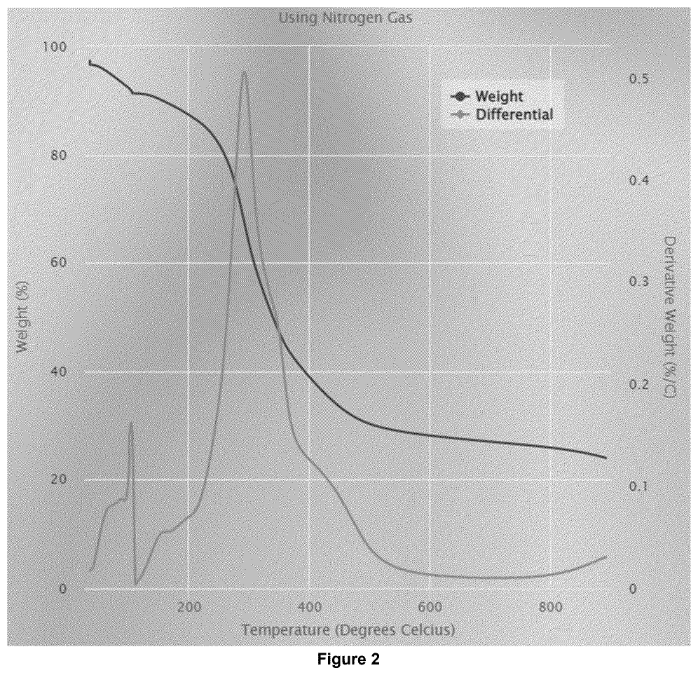

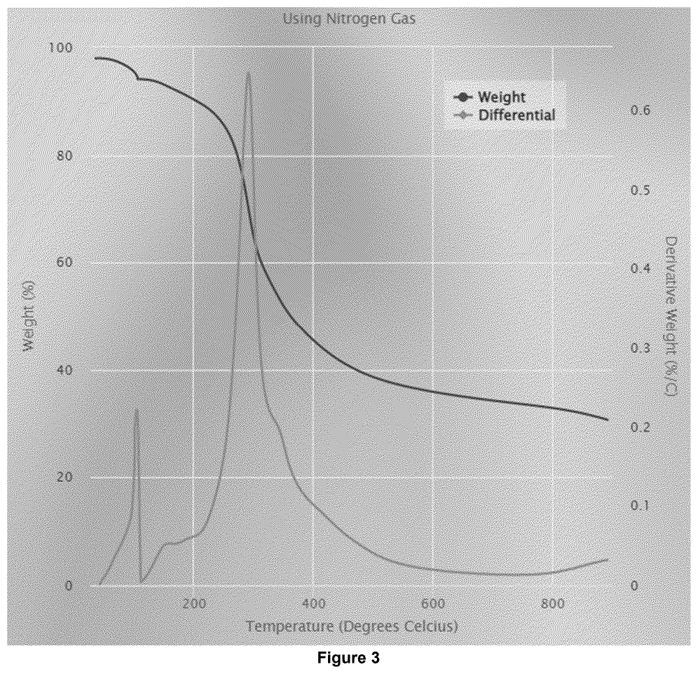

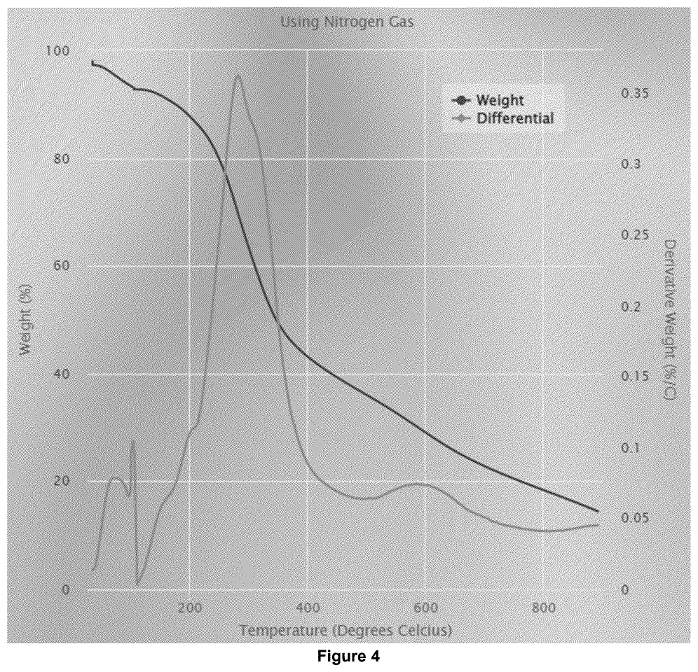

In one embodiment, the edible mycelium ingredients A and B have a thermal stability up to 210 to 220° C. under N₂ atmosphere, preferably up to 220° C. at most. This is seen in the thermogravimetric analysis (TGA), determined at the point wherein all the moisture content is already lost mass wise. In one embodiment, the edible mycelium ingredients C have a thermal stability up to 175 to 185° C. under N₂ atmosphere, preferably up to 190° C. at most. It is observed that up to 1000° C., C is less thermally stable than A, which is slightly less thermally stable than B, as shown in FIGS. 2, 3 and 4.

The texture attributes of the mycelial ingredients are herein analyzed with respect to the shear force, the water holding capacity, water release and the density.

The shear force is influenced by the morphology of the mycelium biomass and its ability to withstand the applied force. In certain embodiments, the shear force of the mycelium ingredient ranges between 1 and 200N, 1 and 150 N, 1 and 100N, 1 and 90N, 1 and 80N, 1 and 70N, 1 and 60N, 1 and 50N, 1 and 40 N, 1 and 30 N, 1 and 25N, 1 and 20N, 1 and 15N, 1 and 10 N, or 1 and 5N. In a preferred embodiment, the shear force of the mycelium biomass is at least 10 N, preferably at least 15N, preferably at least 25 N, preferably ranges between 25 and 150 N, more preferably between 30 and 120N, most preferably between 30 to 105 N.

In certain embodiments the mycelium biomass has a water holding capacity ranging between 1 and 100%, 1 and 90%, 1 and 80%, 1 and 70%, 1 and 60%, 1 and 50%, 1 and 40%, 1 and 30%, 1 and 25%, 1 and 20%, 1 and 15%, 1 and 10%, or 1 and 5%. In a preferred embodiment, the water holding capacity ranges between 20 and 90%, preferably between 30 and 80%, more preferably between 40 and 70%.

In certain embodiments the mycelium biomass has a water release ranging between 1 and 100%, 1 and 90%, 1 and 80%, 1 and 70%, 1 and 60%, 1 and 50%, 1 and 40%, 1 and 30%, 1 and 25%, 1 and 20%, 1 and 15%, 1 and 10%, or 1 and 5%. In another preferred embodiment, the water release of the mycelium ranges between 25 and 70%, more preferably between 30 and 55%.

In certain embodiments the mycelium biomass has a density ranging between 0.1 and 10 g/cm³, 0.1 and 9 g/cm³, 0.1 and 8 g/cm³, 0.1 and 7 g/cm³, 0.1 and 6 g/cm³, 0.1 and 5 g/cm³, 0.1 and 4 g/cm³, 0.1 and 3 g/cm³, 0.1 and 2.5 g/cm³, 0.1 and 2 g/cm³, 0.1 and 1.5 g/cm³, 0.1 and 1 g/cm³, 0.1 and 0.8 g/cm³, or 0.1 and 0.5 g/cm³. In a preferred embodiment, the density of the mycelium biomass ranges between 0.1 and 3, preferably between 0.5 and 2.5, more preferably between 0.5 and 1.5 g/cm³. Preferably the density of the mycelium biomass is around 1 g/cm3.

Texture attributes in the fungal product derived from the mycelial ingredients are influenced by compositional ingredients of the product leading to either a soft or non-soft (i.e., hard) mycelium-based meat analogue or dairy analogue.

In some embodiments, the meat-analogue or the meat-like food product is understood to preferably have a similar consistency or resemblance or taste to the following animal meat in all its forms (breasts, fillets, thighs, ribs, wings, chunks, steaks, etc.), selected from: beef meat, poultry meat, fish meat, chicken meat, duck meat, goose meat, turkey meat, cow meat, pheasant meat, lamb and mutton meat, white meat, pork meat, ham meat, veal meat, deer or venison meat, seafood meat, prawn meat, crab meat, salmon, cod, pangasius, sardines, mussels and oysters.

In a preferred embodiment, soft meat analogues are preferably understood as meat balls. In another preferred embodiment soft meat analogues are preferably meatballs, sausages, fish fingers, tartar, minced meat, meat spreads, processed meat, Mett meat, luncheon meats, foie gras.

In another preferred embodiment, non-soft meat analogues are preferably understood as steak, beef jerky, burger patty, fillet, nugget, salami, whole-cuts, bacon, hot dogs, prosciutto, dried meat, and extruded products.

In another embodiment, the concept of non-soft meat and soft meat may be interchangeable, only if ingredients used to produce a traditionally non-soft meat leads to a softer meat-analogue in terms of consistency compared to the traditional definition.

In a preferred embodiment, soft dairy analogues are preferably understood as cream cheese. In another preferred embodiment soft dairy analogues are preferably understood as cream cheese, cheese spreads, processed cheese, whey cheese, pizza cheese, shredded mozzarella cheese, mozzarella cheese, soft cheese, semi-soft cheese, feta cheese, ricotta cheese, cottage cheese, camembert cheese, Roquefort cheese, Gorgonzola cheese, Brie cheese, blue cheese, Buchette cheese, goat cheese, quark, creams, coffee creamer, whipped creme, sour creme, milk chocolate spreads, margarine, butter, desserts, custard. In another embodiment, non-soft or hard dairy analogue are preferably understood as hard cheeses, semi-hard cheeses, cheddar cheese, parmesan cheese, etc.

In certain embodiments, the cutting strength of the food product ranges between 1 and 100N, 1 and 90N, 1 and 80N, 1 and 70N, 1 and 60N, 1 and 50N, 1 and 40N, 1 and 30N, 1 and 25N, 1 and 20N, 1 and 15N, 1 and 10N, or 1 and 5N. In a preferred embodiment, the cutting strength for soft meat analogues ranges between 1 and 25 N, most preferably between 4 and 8 N. In a preferred embodiment, the cutting strength for hard (meat analogues ranges between 1 and 50 N, most preferably between 10 and 30 N.

In certain embodiments, the hardness of the food product ranges between 1 and 200N, 1 and 150 N, 1 and 100N, 1 and 90N, 1 and 80N, 1 and 70N, 1 and 60N, 1 and 50N, 1 and 40 N, 1 and 30 N, 1 and 25N, 1 and 20N, 1 and 15N, 1 and 10 N, or 1 and 5N. In a preferred embodiment, the hardness for soft meat analogues ranges between 10 and 55 N, preferably 15 and 55 N more preferably between 20 and 50N, most preferably between 20 to 45 N. In a preferred embodiment, the hardness for hard meat analogues ranges between 30 and 100 N, more preferably between 50 and 100N.

In certain embodiments, the springiness of the food product ranges between 1 and 100%, 1 and 90%, 1 and 80%, 1 and 70%, 1 and 60%, 1 and 50%, 1 and 40%, 1 and 30%, 1 and 25%, 1 and 20%, 1 and 15%, 1 and 10%, or 1 and 5%. In a preferred embodiment, the springiness for soft meat analogues ranges between 35 and 85%, preferably between 45 and 80%, more preferably between 50 to 80%, most preferably between 55 and 75%. In a preferred embodiment, the springiness for hard meat analogues ranges between 20 and 70%, preferably between 30 and 60%.

In certain embodiments, the cohesiveness of the food product ranges between 1 and 100%, 1 and 90%, 1 and 80%, 1 and 70%, 1 and 60%, 1 and 50%, 1 and 40%, 1 and 30%, 1 and 25%, 1 and 20%, 1 and 15%, 1 and 10%, or 1 and 5%. In a preferred embodiment, the cohesiveness for soft meat analogues ranges between 15 and 70%, preferably between 20 and 60%, more preferably between 25 to 50%, most preferably between 30 and 45%. In a preferred embodiment, the cohesiveness for hard meat analogues ranges between 20 and 85%, preferably between 30 to 40%.

In certain embodiments, the gumminess of the food product ranges between 1 and 200N, 1 and 190N, 1 and 180N, 1 and 170N, 1 and 170 N, 1 and 160N, 1 and 170N, 1 and 160N, 1 and 150N, 1 and 140N, 1 and 130N, 1 and 120N, 1 and 110N, 1 and 110N, 1 and 90N, 1 and 80N, 1 and 70N, 1 and 60N, 1 and 50N, 1 and 40 N, 1 and 30 N, 1 and 25N, 1 and 20N, 1 and 15N, 1 and 10 N, or 1 and 5N. In a preferred embodiment, the gumminess for soft meat analogues ranges between 1 and 40N, preferably between 3 and 33N, more preferably between 5 to 25N, most preferably between 6 and 21N. In a preferred embodiment, the gumminess for hard meat analogues ranges between 6 and 85N, preferably between 15 and 40N.

In certain embodiments, the chewiness of the food product ranges between 1 and 200N, 1 and 190N, 1 and 180N, 1 and 170N, 1 and 170 N, 1 and 160N, 1 and 170N, 1 and 160N, 1 and 150N, 1 and 140N, 1 and 130N, 1 and 120N, 1 and 110N, 1 and 110N, 1 and 90N, 1 and 80N, 1 and 70N, 1 and 60N, 1 and 50N, 1 and 40 N, 1 and 30 N, 1 and 25N, 1 and 20N, 1 and 15N, 1 and 10 N, or 1 and 5N. In a preferred embodiment, the chewiness for soft meat analogues ranges between 0.3 and 35N, preferably between 1 and 27N, more preferably between 2.5 to 20N, most preferably between 3 and 16N. In a preferred embodiment, the chewiness for hard meat analogues ranges between 1 and 60N, preferably between 4 and 25N.

In certain embodiments, the adhesiveness of the developed meat analogue, ranges between 0 and −100 N·s, 0 and −90 N·s, 0 and −80 N·s, 0 and −70 N·s, 0 and −60 N·s, 0 and −50 N·s, 0 and −40 N·s, 0 and −30 N·s, 0 and −20 N·s, 0 and −10 N·s, 0 and −5 N·s, 0 and −1 N·s, 0 and −0.01 N·s, or 0 and −0.001 N·s. In certain embodiments, the stickiness for the mycelium-based meat analogue ranges between 0 and −0.3 N·s, more preferably between −0.01 and −0.1 N·s, most preferably between −0.02 and −0.05, which is a low stickiness mouthfeel.

In certain embodiments, the firmness of the dairy analogue, such as cream cheese or any mycelium-based spreads, ranges between 1 and 100N, 1 and 90N, 1 and 80N, 1 and 70N, 1 and 60N, 1 and 50N, 1 and 40 N, 1 and 30 N, 1 and 25N, 1 and 20N, 1 and 15N, 1 and 10 N, or 1 and 5N. In another preferred embodiment, the firmness for a soft dairy analogue ranges between 1 and 20 N, more preferably between 5 and 15 N, most preferably between 5 and 10 N. In a preferred embodiment, the firmness for a hard dairy analogue ranges between 20 and 100N, preferably between 20 and 50N, more preferably between 20 to 40N, most preferably between 25 and 35 N.

In certain embodiments, the spreadability of the dairy analogue, such as cream cheese or any mycelium-based spreads, ranges between 1 and 100 N·s, 1 and 90 N·s, 1 and 80 N·s, 1 and 70 N·s, 1 and 60 N·s, 1 and 50 N·s, 1 and 40 N·s, 1 and 30 N·s, 1 and 25 N·s, 1 and 20 N·s, 1 and 15 N·s, 1 and 10 N·s, or 1 and 5 N·s. In another preferred embodiment, the spreadability for a soft dairy analogue ranges between 1 and 20 N·s, more preferably between 10 and 20 N·s. In a preferred embodiment, the spreadability for a hard dairy analogue ranges between 30 and 100 N·s, preferably between 40 and 80 N·s, more preferably between 40 to 70 N·s, most preferably between 45 and 70 N·s.

In certain embodiments, the stickiness of the developed dairy analogue, such as cream cheese or any mycelium-based spreads, ranges between −1 and −100N, −1 and −90N, −1 and −80N, −1 and −70N, −1 and −60N, −1 and −50N, −1 and −40 N, −1 and −30 N, −1 and −25N, −1 and −20N, −1 and −15N, −1 and −10 N, or −1 and −5N. In another preferred embodiment, the stickiness for a soft dairy analogue ranges between −1 and −14 N, more preferably between −3 and −10 N, which is a low stickiness. In a preferred embodiment, the stickiness for a hard dairy analogue ranges between −15 and −100N, preferably between −15 and −50N, more preferably between −15 to −30N, most preferably between −15 and −25 N.

In certain embodiments, the puncture force of the dairy analogue, such as cream cheese or any mycelium-based spreads, leads to a an area under the curve which ranges between 1 and 100 N·s, 1 and 90 N·s, 1 and 80 N·s, 1 and 70 N·s, 1 and 60 N·s, 1 and 50 N·s, 1 and 40 N·s, 1 and 30 N·s, 1 and 25 N·s, 1 and 20 N·s, 1 and 15 N·s, 1 and 10 N·s, or 1 and 5 N·s. In a preferred embodiment, this area for a soft dairy analogue ranges between 1 and 30 N·s, preferably between 5 and 25 N·s, more preferably between 5 to 20 N·s, most preferably between 8 and 18 N·s. In another preferred embodiment, the area for a hard dairy analogue ranges between 40 and 100 N·s, more preferably between 40 and 80 N·s, most preferably between 50 and 60 N·s, which indicates that a higher time and a higher force is needed to puncture the hard dairy analogue.

In a specific exemplary embodiment illustrating the above embodiment, the mycelium of Pleurotus Pulmonarius was cultivated at ambient temperature in a medium selected from three different mediums, namely in a defined medium leading to ingredient A, in a fully synthetic medium leading to ingredient B, or in a natural medium based on an extract from spent grain, characterized by a particle size distribution determined by using a different set of sieves (DIN 10765 mod.) comprising a maximum distribution between 2-4 mm of about 40 wt. % followed by a second maximal distribution between 1-2 mm of about 26 wt. %

The mycelium ingredients of the present invention were then analyzed for its ash content and its elemental composition (carbon C, hydrogen H, nitrogen N, sulfur S, oxygen O) with the oxygen content determined by difference. The ash content of ingredients A, B, and ° C. are 9.39%, 9.73%, and 8.40%. The elemental analysis of C, H, N, O, S analysis of these ingredients leads to,

For A: 44.45% (Carbon C), 5.95% (H), 6.25% (N), 0.32% (S), 33.53% (O); Carbon to nitrogen ratio (mycelium): 7.11.

For B: 43.92% (Carbon C), 5.83% (H), 4.62% (N), 0.26% (S), 35.64% (O); Carbon to nitrogen ratio (mycelium): 9.5.

For C: 46.90% (Carbon C), 6.26% (H), 9.57% (N), 0.42% (S), 28.45% (O); Carbon to nitrogen ratio (mycelium): 4.9.

The lower nitrogen content in sample B indicates a sample richer in fiber content but having a lower protein content, whereas sample C, based on the brewer's spent grain, has the highest nitrogen content indicating a high protein content with a lower fiber content. Sample A is between the two. This indicates that the protein and fiber content can be tailored by tweaking the composition of the medium either synthetically or by using nutritious sidestreams with analyzed elemental compositions, allowing the production of tailored food products, either richer in fiber or in protein.

The Higher Heating Value (HHV, often referred to as the Gross Calorific Value) was determined directly using an oxygen bomb calorimeter as outlined in EN 14918:2009. The Lower Heating Value (LHV, often referred to as the Net Calorific Value) was calculated based on the HHV and the elemental composition of the sample.

For A HHV and LHV are 18.4 MJ/kg (440 Kcal/100 g) and 17.11 MJ/kg (410 Kcal/100 g), respectively.

For B HHV and LHV are 18.88 MJ/kg (450 Kcal/100 g) and 17.61 MJ/kg (420 Kcal/100 g), respectively.

For C HHV and LHV are 19.73 MJ/kg (470 Kcal/100 g) and 18.37 MJ/kg (440 Kcal/100 g), respectively.

The most abundant sugar as a percentage of the total sugar content in the mycelial ingredients after hydrolysis for is A 88% of glucan, for B 92.5% of glucan, and for C 78.6% of glucan (the glucan here is based on all the glucose, including glucose derived from other polysaccharides, such as heteroglycan and/or exopolysaccharide).

Sugars in the water extract or extractives comprise the sugars extracted via water from fresh mycelium ingredients. The water extraction was done with ASE 200 (Accelerated Solvent Extractor) in 11 ml stainless steel extraction cells, wherein deionized water at 100° C., 1500 psi was used (heat time 5 min, static time: 7 min, flush volume: 150%, purge time: 180 sec, static cycles: 3). The total sugars in the water extracts of ingredient A, B, and C are 5.65%, 7%, and 3.26% respectively, from which for A, the sugars in the water extract expressed as the % of the total extract sugars comprises of disaccharides (trehalose 20.1%), hexose sugars (glucose 22%, fructose 0.5%, mannose, 0.1%, galactose 1.4%), pentose sugars (arabinose 0.1%) and sugar alcohols (mannitol 55%, sorbitol 0.7%). For B, the sugars in the water extract expressed as the % of the total extract sugars comprises of disaccharides (trehalose 19.2%), hexose sugars (glucose 43.3%, fructose 0.4%, galactose 0.1%), pentose sugars (arabinose 0.1%) and sugar alcohols (mannitol 35.7%, sorbitol 1%). For C, the sugars in the water extract expressed as the % of the total extract sugars comprises of disaccharides (trehalose 46%), hexose sugars (glucose 16%, galactose 0.1%), pentose sugars (arabinose 0.03% and xylose 0.15%) and sugar alcohols (mannitol 22.7%, arabinitol 13.5%, xylitol 1%, sorbitol 0.64%).

In one embodiment, the water extract of the mycelium ingredient A contains at least 50 wt. % mannitol out of the extracted sugars (i.e. with respect to the total sugar content), which is the highest amount of sugar extracted compared to others.

In one embodiment, the water extract of the mycelium ingredient B contains at least 40 wt. % glucose out of the extracted sugars (i.e. with respect to the total sugar content), which is the highest amount of sugar extracted compared to others.

In one embodiment, the water extract of the mycelium ingredient C contains at least 40 wt. % trehalose out of the extracted sugars (i.e. with respect to the total sugar content), which is the highest amount of sugar extracted compared to others.

The flavors in terms of the known flavor 5′ nucleotides (5′NMP) in g/kg, which include 5′-inosine monophosphate (IMP), 5′-guanosine monophosphate (GMP) and 5′-adenosine monophosphate (AMP) were measured. Ingredient A shows a good richness of 5′NMP of 3.56 g/kg compared to B showing 1.78 g/kg. However, the usage of spent grain extract led to 10-fold increase in ingredient C compared to C, leading to a 5′NMP of 10.43 g/kg. Additionally, ingredient C showed the highest concentration of Uridine monophosphate (UMP) of 3.56 g/kg compared to 1.26 g/kg and 0.71 g/kg for A and B, respectively.

Without any extra ribonucleic acid (RNA) reduction step in the production process of the ingredients via additional heating or pH treatment, the method of this invention leads to an inherent RNA level of the ingredients equal or below 2 wt. %, specifically 1.88 wt. % for A, 1.65 wt. % B and 2 wt. % for C in one embodied example.

Free bases comprise cytosine, uracyl, guanine, hypoxanthine, and adenine. The overall free bases measured for mycelium ingredient A is 0.62 g/kg compared to 0.48 g/kg for ingredient B and 1.3 g/kg for ingredient C, wherein for ingredient A, adenine and uracyl are observed to be the highest, 0.28 g/kg and 9.18 g/kg, respectively, for ingredient B the same observation for ingredient

A but with a lower concentration, 0.17 g/kg and 0.13 g/kg, respectively, where for ingredient C a higher amount of adenine, uracyl and cytosine is observed to be, 0.54 g/kg, 0.22 g/kg, and 0.49 g/kg, respectively.

Free purine nucleosides comprising of guanosine, inosine and adenosine were measured to be the highest for ingredient A (2.77 g/kg), followed by ingredient C (2.24 g/kg) and then ingredient B (1.11 g/kg).

The chitin content for the ingredients was about 7.6 wt. % for A, 6.9 wt. % for B. and 7.5 wt. % for C. The ergothioneine contents were about 91 mg/kg for A, 127 mg/kg for B, and 433 mg/kg. An ergosterol content of 2.35 mg/g dry weight was determined for sample A, 2.24 mg/g dry weight for sample B, and 5.56 mg/g dry weight for sample C (knowing that the oyster mushrooms are reported to have 4.4 mg/g dry weight, in particular for P. pulmonarius fruiting bodies which were grown on three forestry wastes (pine, poplar, and honeysuckle rattan) have an ergosterol content between 2.9 and 3.3 mg/g, which is less than the content of ergosterol found in the mycelium ingredient C, showing a higher enrichment compared to the mushroom fruiting body itself).

All ingredients A, B, and C show an umami amino acid of at least about 20 wt. %, more specifically, 21.48 wt. % for A, 19.5 wt. % for B, and 22.64 wt. % for C.

All ingredients A, B, and C show a BCAA content of at least about 20 wt. %, more specifically, 21.73 wt. % for A, 23.1 wt. % for B, and 19.6 wt. % for C.

All ingredients A, B, and C show content of the essential amino acids of at least about 40 wt. %, more specifically, 40.81 wt. % for A, 40.52 wt. % for B, and 39.61 wt. % for C.

The total amino acid content in mg/g is richest in C (361.67 mg/g), followed by A (268.85 mg/g), followed by B (163.43 mg/g).

The mycelium ingredient A is grown on a medium containing a carbon to nitrogen ratio (medium) of between 16 and 18, preferably of about 17. The mycelium ingredient B is grown on a medium containing a carbon to nitrogen ratio (medium) of between 19 and 21, preferably of about 20. Mycelium ingredient B is grown on a medium containing a carbon to nitrogen ratio (medium) of between 12 and 14, preferably of about 13.

The insoluble fiber content increases from C to A to B from around 23 wt. %, to around 34 wt. % to around 54 wt. %, while the protein content from C to A to B decreases from around 60 wt. % to around 39 wt. % to around 32 wt. %. This shows the relationship discussed earlier between proteins and fibers, that they are inversely proportional and can be controlled by varying the fermentation medium composition. However, this trend may not always present as reported in the following study on a submerged fermentation of Pleurotus tuber-regium, where a lower C/N ratio in the medium lead to a higher total dietary fiber in the mycelial cell wall (Food Chemistry 85 (2004) 101-105.), while the observed trend of this invention shows that the fiber content increases with increasing the C/N ratio This indicates that such trends may highly depend on the medium composition such as carbon source, or nitrogen source, fermentation process conditions such as pH and stirring speed, the inoculum, and/or the other factors.

The fat content of ingredient A is about 3 wt. %. The fat content of ingredient B is about 2 wt. % The fat content of ingredient C is about 7 wt. %. This fat comprises of saturated fatty acids, monosaturated fatty acids, polyunsaturated fatty acids and trans fatty acids, as shown in the table below. It is worth noting that the content of omega-6 fatty acid (linoleic acid) is 0.77 wt. for A, 0.46 wt. % for B and 3.5 wt. % for C. All the ingredients show a calculated carbohydrate content of less than 0.1 wt. %. B is the richest in glucans total, with a value of 31 wt. %. The mycelium ingredient A has a total glucans value of about 26 wt. %. The mycelium ingredient C has a total glucans value of about 17 wt. %.

It was calculated that the EUC for the mycelium ingredients is 302% for A, 34% for B, and 2892% for C. In a separate study, P. pulmonarius fruiting bodies were grown on three forestry wastes (pine, poplar, and honeysuckle rattan), showing EUC values of P. pulmonarius fruiting bodies between 72.31% and 116.73% (Food Chemistry 397 (2022) 133714). The disclosed EUC values of mycelium ingredients A, B, and C of this invention are higher than this reported value for a fruiting body of the same fungal strain, and taking ingredient C as an example, the difference is 25 to 40-fold higher for ingredient C compared to the reported range of P. pulmonarius fruiting bodies. Knowing also that the highest value of EUC of a mushroom fruiting body was reported to be 4465% for (Volvariella volvacea), which is lower by a factor of 2.35 compared to ingredient C.

It is be noted that reported mycelia (not fruiting body) had lower EUC values than the fruiting bodies for Pleurotus eryngii (30.9% EUC mycelia vs.116% fruiting body), Agrocybe aegerita (19.2% EUC mycelia vs. 322% EUC fruiting body), and Lentinus edodes (16.7% EUC mycelia vs. 99.75% EUC fruiting body) (https://doi.org/10.1080/10942912.2015.1089891), which highlights the importance and the unique taste that the mycelium ingredients of this invention possess via submerged fermentation. When looking further into the literature, the EUC for mycelia was reported for the following species: Termitomyces albuminosus 460% Wen (2003), Grifola frondosa 375% Wen (2003), Morchella esculenta 363% Wen (2003), Hypsizygus marmoreus 128% Lee (2003), Cordyceps militaris 124% Chang et al. (2001), Pleurotus citrinopileatus 37.1% Huang (2003), Antrodia camphorata 21.2% Chang et al. (2001), Ganoderma tsugae 19.4% Tseng et al. (2004), Agaricus brasiliensis 1.92% Chang et al. (2001), Pleurotus eryngii via submerged fermentation 9 to 144% (All these references are disclosed accordingly in Mau et al., International Journal of Medicinal Mushrooms, Vol. 7, pp. 119-125 (2005))) which can be all taken as a basis to show that the mycelium ingredients of this invention exhibit an unprecedented high-profile umami tase to be used in the food industry and food products, such as dairy-analogues, meat analogues, or other food products.

Additionally, comparing mycelium ingredient A and C with unwashed mycoprotein product from Quorn, according to the data disclosed in WO2021234349 (Table 1 and 2), a maximum EUC could be approximated to be at most 148% (Knowing that the real value of EUC should be lower than 148% since in this calculation the total amino acids was used instead of the free amino acids due to lack of data).

It is interesting to note that the sample grown on the sidestream has the richest values in terms of total amino acid content, umaminess, ergothionene content, ergsterol content, and other properties, which shows a big advantage in using natural sidestreams or waste streams to produce richer foods, by at the same time, upcycling waste streams from other value chains, which contributes to sustainability. However, these ingredients differ in their properties, which make them suitable for different applications. As an example, the usage of ingredient B for sports supplements as it is low in fat and high in fiber content and in BCAAs amino acids with a sufficient protein content.

The present invention is also concerned with methods of producing edible dairy substitute, meat substitute and fish/seafood substitute products using the fungal ingredients of the present invention.

In certain embodiments, the mycelium ingredients can be used in the form of wet biomass (as-is), washed biomass, dried biomass, grinded to a powder with a specific particle size distribution, whether with fine particle size or coarse, or medium particle size.

A method for producing a soft or hard meat analogue composition comprising of at least one of the mycelium ingredients from at least one fungal strain, comprising the methods of producing the respective mycelium ingredient and further comprising the step of preparing such meat analogue composition by mixing of at least one of the mycelium ingredient from at least one fungal strain with a composition comprising of at least one protein rich ingredient, at least one plant-based lipid rich ingredient, and optionally at least one compositional ingredient.

In one embodiment, 50 to 95 wt. % of mycelium ingredient (edible fibrous mycelium) is blended and then added to 1 to 40 wt. % at most from each of the following ingredients: canola oil, salt, egg white and wheat gluten, then optionally 1 to 40 wt. % is added at most from each of the following ingredients: methylcellulose, hydrocolloids, texturized vegetable proteins, starch-based ingredients, flavor components. The obtained dough is then shaped via a forming machine and/or an extruder or a combination thereof, to have the final product as meat balls or sausages or extruded product.

A method for producing a dairy analogue composition comprising of at least one of the mycelium ingredients from at least one fungal strain, comprising the methods of producing the respective mycelium ingredient and further comprising the step of preparing such a dairy analogue composition by (1) forming a slurry comprising at least one of the mycelium ingredients with a composition comprising of at least one protein rich ingredient, at least one plant-based lipid rich ingredient, and at least one compositional ingredient, and (2) mixing the slurry with at least one compositional ingredient, specifically a texturizing agent or thickener or a carbohydrate-rich ingredient.

In one embodiment, the method of producing an edible dairy substitute product comprises no extra acidification step, but rather an acidification through its formulation:
(1) Homogenizing 10-50 wt. % of edible fibrous mycelium derived from submerged fermentation from at least one fungal strain with about 55 wt. % potable water, about 10 to 45 wt. % of plant derived fat component, 2 wt. % at most of sodium chloride source, 2 wt. % at most of yeast flakes, 5 wt. % at most of plant derived mono- or disaccharide source, 5 wt. % natural plant derived acidity source
(2) Heating the homogenized slurry under constant blending for a maximum of 60 seconds at a temperature of 75 to 95° C. To coagulate the homogenized slurry, a beforehand prepared coagulation agent solution is administered during this precedingly circumscribed heating step as soon as the temperature has reached 25° C. to 40° C. This coagulation agent solution comprises of 15 wt. % at most of each of the following: water, a wheat starch as well as a hydrolyzed corn starch.
(3) Removing the obtained slurry from the heat source and subsequently allowing the mixture to settle to room temperature (21° C.). Once room temperature is reached, the coagulated slurry is stored in a dark and cool environment, preferably between 4° C. to 7° C.

In a further embodiment, the method of producing an edible non-animal dairy substitute product comprises an extra acidification step as follows:
(1) Homogenizing 10-50 wt. % of edible fibrous mycelium derived from submerged fermentation from at least one fungal strain with about 55 wt. % potable water, about 10 to 45 wt. % of plant derived fat component, 2 wt. % at most of sodium chloride source, 2 wt. % at most of yeast flakes, 5 wt. % at most of plant derived mono- or disaccharide source.
(2) Heating the homogenized slurry under constant blending for a maximum of 60 seconds at a temperature of 75 to 95° C. To coagulate the homogenized slurry, a beforehand prepared coagulation agent solution is administered during this precedingly circumscribed heating step as soon as the temperature has reached 25° C. to 40° C. This coagulation agent solution comprises of 15 wt. % at most of each of the following: water, a wheat starch as well as a hydrolyzed corn starch.
(3) Removing the obtained slurry from the heat source and subsequently allowing the mixture to settle to room temperature (21° C.). Once the obtained mixture reaches a temperature of 40° C. or less, add 0.1 g to 0.25 g of acid-forming bacteria, in particular lactic acid bacteria to induce microbial acidification. To provide a hospital environment for the microorganisms, the sample is placed for 150 minutes into a controlled temperature environment of 28° C. (e.g., water bath, or incubator). Depending on intensity of fermentation, sample can be placed from 1 to 6 hours at a temperature of 20 to 45° C. After the microbial fermentation is completed, the coagulated slurry is stored in a dark and cool environment, preferably between 4° C. to 7° C.

In a third embodiment, the mycelium from one fungal strain is mixed with a mycelium of another fungal strain or with algae, bacteria, plant cells, archaea cells, fat cells or a combination thereof. In a preferred embodiment, the mycelium from one fungal strain is partly replaced by a mycelium of another fungal strain, to produce a dairy or meat analogue, in a ratio of the overall mycelium content ranging between: 1:100, 10:90, 20:80, 30:70, 40:60, 50:50, 60:40, 70:30, 80:20, 90:10, or 100:1. In a preferred embodiment, if a second mycelium component exists, then the ratio of the two mycelium components is 20:80, preferably 50:50, more preferably 40:60, most preferably 30:70. In one embodiment, Pleurotus Pulmonarius edible fibrous mycelium is mixed with Morchella rufobrunnea edible fibrous mycelium, wherein 60-90 wt. % of total mycelium would be from Pleurotus Pulmonarius mycelium, and 10-40 wt. % of the total mycelium ingredients would be from Morchella rufobrunnea mycelium (edible fibrous mycelium). In another embodiment, Pleurotus Pulmonarius edible fibrous mycelium is mixed with Morchella rufobrunnea edible fibrous mycelium, wherein 60-90 wt. % of total mycelium would be from Morchella rufobrunnea mycelium, and 10-40 wt. % of the total mycelium ingredients would be from Pleurotus Pulmonarius mycelium (edible fibrous mycelium).

In another embodiment, the same method is extended to at least three fungal strains.

Thereby obtaining an edible cheese substitute product selected from the group comprising an edible substitute product for whey cheese, cream cheese, medium-hard cheese, hard-cheese, and soft-mould cheese. Thereby obtaining an edible cheese substitute product being cream cheese.

Adding a texturizing agent is an optional step depending on the water content of the homogenized mycelium mass obtained in step a). If the water content is similar to the water content of the substitute product to be produced, then adding a texturizing agent is not necessary to obtain the desired texture.

In a preferred embodiment, the product obtained with this method is an edible fresh cheese substitute product or a creme cheese. It can also be an edible cheese substitute product selected from the group comprising an edible substitute product for whey cheese, cream cheese, medium-hard cheese, hard-cheese, and soft-mould cheese, which can be obtained without traditional coagulation steps if the water content of the homogenized mycelium mass is suitably adjusted to be sufficiently low. The coagulation steps of traditional cheese making are performed to get rid of the excess water provided by the starting material, which is milk. By having a starting material, i.e., the homogenized mycelium mass, which has a significantly lower water content compared to milk, these coagulation steps of traditional cheese making are not necessary.

In the method of producing an edible dairy substitute product for fresh cheese, whey cheese, cream cheese, medium-hard cheese, hard-cheese, and soft-mould cheese, the edible plant-based fat component is preferably selected from coconut oil, sunflower oil, rapeseed oil, palm oil, cotton seed oil, olive oil, canola oil, algae oil, or oleaginous yeast-derived oils.

In further preferred embodiments, specifically when the edible dairy substitute product is a substitute product for yoghurt or cheese, the edible dairy substitute product further comprises an edible plant-, or algae-based fat component, a fat component derived from fungi or yeast. The fat component is preferably in the range of up to 60 wt. %, preferably up to 25 wt. %, and most preferably from 1 wt. % to 5 wt. %. The amount of the fat component can be adjusted depending on the product to be substituted.

The edible dairy substitute product of the present invention preferably comprises an edible fibrous mycelium mass content of between 1 wt. % and 99 wt. %, preferably between 10 wt. % and 90 wt. %, preferably between 1 wt. % and 65 wt. %, most preferably between 10 and 50 wt. %. When discussing the content of edible fibrous mycelium in the product of the invention, reference is preferably made to a wt. % in the product containing water (i.e., there is no normalisation to the dry mass content).

Preferably, the edible fibrous mycelium in the dairy-analogue product has an insoluble fiber content of at least 20 wt. %, more preferably at least 30 wt. %, more preferably at least 40 wt. %. Accordingly, it is preferred that the insoluble fiber content is between 20 wt. % and 60 wt. %. More preferably, said edible fibrous mycelium has an insoluble fiber content of between 40 wt. % and 60 wt. %. Even more preferably, said edible fibrous mycelium has an insoluble fiber content of between 40 wt. % and 55 wt. %. Even more preferably, said edible fibrous mycelium has an insoluble fiber content of between 40 and 50 wt. %. Most preferably, said edible fibrous mycelium has an insoluble fiber content of about 45 wt. %. Most preferably, said edible fibrous mycelium has an insoluble fiber content of at least 50 wt. %. It is to be understood that the weight as referred to herein relates to dry mass of said mycelium. In one embodiment, the edible fibrous mycelium has an insoluble fiber content of at least 40 wt. %, preferably of at least 50 wt. %, more preferably of at least 60 wt. %.

The protein content or the fiber content of the mycelium mass can be adjusted by choosing fermentation conditions, which control the protein or fiber content of the obtained mycelium mass. For example, the ratio between carbon and nitrogen source in the medium can be altered, such as providing nitrogen in different grades of excess, as envisaged in this invention.

In some embodiments, specifically when the edible dairy substitute product is a substitute product for yoghurt or cheese, the edible dairy substitute product can further comprise a texturizing agent, such as agar-agar, edible starch, guar gum, locust bean gum, wheat gluten, cellulose or derivatives of it. The texturizing agents give structure to the product, thereby making it suitable as a substitute product for yoghurt or cheese. The texturizing agent is however an optional component for the edible dairy substitute product of the present invention. If the water content of the mycelium homogenized with water is kept low by using a pressed or partially dried mycelium having a desired water content, the resulting mass will have the desired structure similar to that of yoghurt or cheese without the need of adding an external texturizing agent. The skilled person will know how to suitably adjust the water content of the mycelium mass to the substitute product to be produced.

In one embodiment, the acidification through ingredients is done via homogenizing 10-50 wt. % of Pleurotus Pulmonarius mycelium (edible fibrous mycelium) with about 55 wt. % potable Water, about 0 to 7 wt. % of cashew nuts (plant derived fat component), 10 to 40 wt. % of Cocos nucifera oil (plant derived fat component), 2 wt. % at most of table salt (sodium chloride source), 2 wt. % at most of yeast flakes, 5 wt. % at most of sucrose (plant derived saccharide source), 5 wt. % at most of lemon juice (natural plant derived acidity source), 5 wt. % at most of citric acid. Under constant blending, heating the homogenized slurry for a maximum of 60 seconds at a temperature of 75 to 95° C. Administering a beforehand prepared coagulation agent during this precedingly circumscribed heating step as soon as the temperature has reached 25° C. to 40° C. This coagulation agent solution comprises of 15 wt. % at most of each of the following: water, a wheat starch as well as a hydrolyzed corn starch, removing the obtained slurry from the heat source and subsequently allowing the mixture to settle to room temperature (21° C.). Once room temperature is reached, the coagulated slurry is stored in a dark and cool environment preferably between 4° C. to 7° C., to obtain the mycelium-based creme cheese product.

In one embodiment, the acidification through microbial fermentation is done via homogenizing 10-50 wt. % of Pleurotus Pulmonarius mycelium (edible fibrous mycelium) with about 55 wt. % potable Water, about 0 to 7 wt. % of cashew nuts (plant derived fat component), 10 to 40 wt. % of Cocos nucifera oil (plant derived fat component), 2 wt. % at most of table salt (sodium chloride source), 2 wt. % at most of yeast flakes, 5 wt. % at most of sucrose (plant derived saccharide source). Under constant blending, heating the homogenized slurry for a maximum of 60 seconds at a temperature of 75 to 95° C. Administering a beforehand prepared coagulation agent during this precedingly circumscribed heating step as soon as the temperature has reached 25° C. to 40° C. This coagulation agent solution comprises of 15 wt. % at most of each of the following: water, a wheat starch as well as a hydrolyzed corn starch, removing the obtained slurry from the heat source and subsequently allowing the mixture to settle to room temperature (21° C.). Once the obtained mixture reaches a temperature of 40° C. or less, adding 0.1 g to 0.25 g of acid-forming bacteria, in particular lactic acid bacteria to induce microbial acidification. Placing the sample for 150 minutes into a controlled temperature environment of 28° C. (e.g., water bath, or incubator). Depending on intensity of fermentation, sample can be placed from 1 to 6 hours at a temperature of 20 to 45° C. After the microbial fermentation is completed, the coagulated slurry is stored in a dark and cool environment, preferably between 4° C. to 7° C., to obtain the mycelium-based creme cheese product.

The above-mentioned adjustability of the composition of the mycelium mass of the present invention provides the advantage of versatility, wherein the composition of the mycelium mass can be easily adjusted to the requirements of the substitute product to be obtained. If the protein content of the mycelium mass is increased, the amount of mycelium mass in the substitute product can be decreased without changing the protein content of the substitute product, for example. If the substitute product is a certain type of cheese, which is characterized by certain amounts of calcium, phosphorus, and/or zinc, then these nutrients can be provided by the mycelium mass itself, obviating the need of supplementing the substitute product with additional nutrients externally. This decreases the costs of the production method and of the obtained product due to easier handling, fewer production steps, and decreased costs for raw material for the purpose of producing edible dairy substitute products with a clean label. Most importantly, the nutrients from the mycelia are bioavailable making the metabolic digestion predictable and easy, especially compared to products, which comprise nutrients supplemented externally.

In one embodiment, this mix of at least two mycelium ingredients is used to produce a dairy analogue. In another embodiment, this mix of at least two mycelium ingredients is used to produce a meat analogue. In a preferred embodiment, this mix of at least two mycelium ingredients is used to produce a dairy and a meat analogue. In a preferred embodiment, this mix of at least two mycelium ingredients is used to produce a fish analogue. In one embodiment, this mix of mycelium ingredients is used to produce other food products defined above.

In a further embodiment, the method of producing an edible vegetarian meat substitute product comprises of
(1) Blending 1 to 99 wt. %, preferably 50 to 95 wt. % of the edible mycelium ingredient and adding it to a mix of at least one protein rich ingredient and at least one plant-based lipid rich ingredient.
(2) Optionally adding at least one compositional ingredient, such ingredients comprise of methylcellulose, hydrocolloids, texturized vegetable proteins, starch-based ingredients, fiber-rich ingredients, and flavor components for seasoning.
(3) Shaping the obtained dough via an extruder and/or forming machine to have the final product as meat balls or sausages or extruded products, wherein step 3 comprises of:
(i) cold extruding the obtained dough into a rope with a thickness of 1.5 to 5 cm, preferably 1.5 to 3.5 cm by a cold extruder or a vacuum filler that fills the forming machine
(ii) Forming the product to the desired shape
(iii) Alternative to step (ii), filling casings in case of a sausage product
(iv) Boiling the product in water at a temperature ranging between 60 to 100° C. for a duration of 1 to 30 minutes to increase the mycelium product texture due to protein denaturation and product shelf-life stability
(v) Alternatively, step (iv) may be replaced by deep-frying followed by steam or hot air cooking of the product at temperature ranging between 5° and 150° C.
(vi) Storing the final product in a freezer

The edible meat substitute product of the present invention preferably comprises an edible fibrous mycelium mass content of between 1 wt. % and 99 wt. %, preferably between 5 wt. % and 99 wt. %, preferably between 10 wt. % and 95 wt. %, preferably between 20 and 95 wt. %., more preferably between 40 and 95, most preferably between 60 and 95 wt. %, wherein preferably the edible fibrous mycelium in the meat-analogue product has an insoluble fiber content between 20 and 60 wt. %, preferably between 40 and 50 wt. %, preferably about 45 wt. % In another preferred embodiment, the insoluble fiber used in a meat analogue is about 35 wt. %. In another preferred embodiment, the insoluble fiber used in a meat analogue is about 25 wt. %. In another preferred embodiment, the insoluble fiber used in a meat analogue is about 45 wt. %. In one embodiment, the edible fibrous mycelium in the meat analogue has an insoluble fiber content of at least 20 wt. %, preferably of at least 30 wt. %, preferably of at least 45 wt. %, preferably of at least 50 wt. %.

The protein content or the fiber content of the mycelium mass can be adjusted by choosing fermentation conditions, which control the protein or fiber content of the obtained mycelium mass. For example, the ratio between carbon and nitrogen source in the medium can be altered, such as providing nitrogen in different grades of excess, as envisaged in this invention.

In one embodiment, the at least one plant-based lipid rich ingredient constitutes at most 40 wt. %, at most 20 wt. %, at most 15 wt. %, at most 10 wt. %, at most 9 wt. %, at most 8 wt. %, at most 7 wt. %, at most 6 wt. %, at most 5 wt. %, at most 4 wt. %, at most 3 wt. %, at most 2 wt. %, or at most 1 wt. % of the overall recipe of the meat-analogue composition. In a preferred embodiment, the at least one plant-based lipid rich ingredient of the meat-analogue composition is between 1 and 40 wt. %, preferably between 1 and 20 wt. %, more preferably between 1 and 10 wt. %

In one embodiment, the at least one protein rich ingredient of the meat-analogue composition constitutes each at most 95 wt. %, at most 80 wt. %, at most 60 wt. %, at most 40 wt. % at most 20 wt. %, at most 15 wt. %, at most 10 wt. %, at most 9 wt. %, at most 8 wt. %, at most 7 wt. %, at most 6 wt. %, at most 5 wt. %, at most 4 wt. %, at most 3 wt. %, at most 2 wt. %, or at most 1 wt. % of the overall recipe of the meat-analogue composition. In a preferred embodiment, the at least one protein rich ingredient of the meat-analogue composition is each between 0.1 and 50 wt. %, preferably between 0.1 and 30 wt. %, more preferably between 0.1 and 20 wt. %.

In one embodiment, the at least compositional ingredient of the meat-analogue composition constitutes each at most 80 wt. %, at most 60 wt. %, at most 40 wt. % at most 20 wt. %, at most 15 wt. %, at most 10 wt. %, at most 9 wt. %, at most 8 wt. %, at most 7 wt. %, at most 6 wt. %, at most 5 wt. %, at most 4 wt. %, at most 3 wt. %, at most 2 wt. %, or at most 1 wt. % of the overall recipe of the meat-analogue composition. In a preferred embodiment, the at least one compositional ingredient of the meat-analogue composition is each between 0.1 and 50 wt. %, preferably between 0.1 and 30 wt. %, more preferably between 0.1 and 20 wt. %.

In one embodiment, the protein rich ingredient comprises wheat gluten. In one embodiment, the protein rich ingredient comprises wheat gluten and egg white. In one embodiment, the compositional ingredient comprises flavor components. In one embodiment, the compositional ingredient comprises flavor components and starch-based ingredient. In one embodiment, the compositional ingredient comprises flavor components, starch-based ingredient, and methycellulose. In one embodiment, the compositional ingredient comprises flavor components, and methycellulose. In one embodiment, the compositional ingredient comprises flavor components, starch-based ingredient, and hydrocolloids. In one embodiment, the compositional ingredient comprises flavor components, and hydrocolloids. In one embodiment, the compositional ingredient comprises flavor components, starch-based ingredient, and texturized vegetable proteins. In one embodiment, the compositional ingredient comprises flavor components, and texturized vegetable proteins. In one embodiment, the compositional ingredient comprises flavor components, and fiber-rich ingredients. In one embodiment, the compositional ingredient comprises flavor components, fiber-rich ingredients, and starch-based ingredient. In one embodiment, the compositional ingredient comprises flavor components, starch-based ingredient, fiber-rich ingredients, and methycellulose. In one embodiment, the compositional ingredient comprises flavor components, and methycellulose, fiber-rich ingredients. In one embodiment, the compositional ingredient comprises flavor components, starch-based ingredient, fiber-rich ingredients, and hydrocolloids. In one embodiment, the compositional ingredient comprises flavor components, fiber-rich ingredients, and hydrocolloids. In one embodiment, the compositional ingredient comprises of components, starch-based ingredient, fiber-rich ingredients, and texturized vegetable proteins. In one embodiment, the compositional ingredient comprises flavor components, fiber-rich ingredients, and texturized vegetable proteins. In one embodiment, the compositional ingredient comprises methylcellulose, hydrocolloids, texturized vegetable proteins, starch-based ingredients, fiber-rich ingredients, and flavor components for seasoning. In one embodiment, the flavor components, understood to be under compositional ingredients as well, comprise of at least one of the following ingredients: salt, pepper, garlic, onions, mushroom fruiting body pieces, ginger, turmeric, curry, sugars (i.e., sucrose, glucose, mono- or disaccharides), oils, lemon juice, orange juice, herbs and spices, yeast flakes). In one embodiment, the fiber-rich ingredients comprise at least one fiber rich ingredient selected from the following ingredients: grain-based flours, grain-based starches, legume-based starches, fruit-based fibers, polysaccharides, starch-based ingredients, psylium husk, inulin, wheat starch, and corn starch. It is be noted that fiber-rich ingredients may also be a starch or a carbohydrate-rich ingredient, as a starch-based ingredients or carbohydrate-rich ingredients may be also a fiber-rich ingredient.

In one embodiment, a method to produce the same meat analogue product by making it vegan comprises of replacing the egg white with more mycelium ingredient of this invention, wherein the dry mycelium ingredient would increase by 20 wt. %, 19 wt. %, 18 wt. %, 17 wt. %, 16 wt. %, 15 wt. %, 14 wt. %, 13 wt. %, 12 wt. %, 11 wt. %, 10 wt. %, 9 wt. %, 8 wt. %, 7 wt. %, 6 wt. %, 5 wt. %, 4 wt. %, 3 wt. %, 2 wt. %, or 1 wt. %. In a preferred embodiment the egg white is replaced by at most 15 to 20 wt. %, preferably by at most 7 to 10 wt. % of dry mycelium ingredient. This is mainly dictated by the quality and type of biomass to preferably replace any milk-derived or vegetarian products to make such product vegan.

In one further embodiment, in the method to produce the vegan meat analogue, 1 wt. % of egg white is preferably replaced with or equivalent to about 20 wt. %, 19 wt. %, 18 wt. %, 17 wt. %, 16 wt. %, 15 wt. %, 14 wt. %, 13 wt. %, 12 wt. %, 11 wt. %, 10 wt. %, 9 wt. %, 8 wt. %, 7.5 wt. % 7 wt. %, 6 wt. %, 5.5 wt. %, 5 wt. %, 4 wt. %, 3.5 wt. %. 3 wt. %, 2.5 wt. %, 2 wt. %, 1.5 wt. %, or 1 wt. % dry mycelium biomass. In a preferred embodiment, in the method to produce the vegan meat analogue, 1 wt. % of egg white is preferably replaced with or equivalent to at most 10 wt. %, preferably by at most 5%, more preferably by an equivalent of 2.5 wt. % to 4 wt. %, most preferably by an equivalent of 1.5 to 2 wt. % of dry mycelium ingredient.

In a preferred embodiment, in the method to produce the vegan soft meat analogue, 1 wt. % of egg white is preferably replaced with or equivalent to at most 10 wt. %, preferably by at most 5%, more preferably by an equivalent of 2.5 to 4 wt. %, most preferably by an equivalent of 1.5 to 2 wt. % of dry mycelium ingredient, wherein the cutting strength of the original vegetarian product and that of the vegan product is in the same range of 1 and 25 N, most preferably between 4 and 8 N.

As an example, for a mycelium-based meatball composition, the same cutting strength of 4.8 N is achieved when replacing each 1 wt. % of egg white with about 1 to 2 wt. %, preferably about 1.5 wt. % of dry biomass. Changing such proportions will lead to a different texture, i.e., a different cutting strength.

In a preferred embodiment, in the method to produce the vegan hard meat analogue, 1 wt. % of egg white is preferably replaced with or equivalent to at most 10 wt. %, preferably by at most 5%, more preferably by an equivalent of 2.5 to 4 wt. %, most preferably by an equivalent of 1.5 to 2 wt. % of dry mycelium ingredient, wherein the cutting strength of the original vegetarian product and that of the vegan product is in the same range of 1 and 50 N, most preferably between 10 and 30 N.

In another embodiment, 50 to 95 wt. % of Pleurotus pulmonarius mycelium or any edible fibrous mycelium is blended and then added to 1 to 40 wt. % at most from each of the following ingredients: canola oil, salt, and wheat gluten. The missing egg white is accounted for by the addition of additional dry mycelium, with an equivalence ratio of 1 to 1.4, respectively. Optionally add 1 to 40 wt. % at most from each of the following ingredients: methylcellulose, hydrocolloids, texturized vegetable proteins, starch-based ingredients, and flavor components for seasoning. The obtained dough is then shaped via a forming machine and/or an extruder or a combination thereof, to have the final product as meat balls or sausages or extruded product.

In a further embodiment, the obtained dough or formulation may undergo an extrusion process, before or after forming, if forming is required, to form the meat-product or other extrudable food products, wherein this extrusion is either a cold extruder, low-temperature extrusion or a high-temperature extrusion.

In one embodiment, a cold extrusion is adopted, wherein the extrusion temperature preferably ranges between −20 to ˜10° C., preferably between −10 and 0° C., preferably between 1 and 20° C., preferably between 4 and 15° C., preferably between 1° and 15° C., preferably between 15 and 25° C., preferably between 25 and 40° C., preferably between 4° and 100° C., preferably between 4° and 80° C., preferably between 50 and 75° C. at a pressure ranging between 1 and 100 bar, more preferably 1 and 70 bar. In a preferred embodiment the extrusion takes place at frozen conditions temperature or at cold temperature or at room temperature, preferably at a temperature of −20° C. at most, more preferably between 1 and 20° C., most preferably between 4 and 15° C. at a pressure ranging between 1 and 100 bar, more preferably 1 and 70 bar.

In one embodiment, the forming is adopted, wherein the forming temperature preferably ranges between 1 and 20° C., preferably between 4 and 15° C., preferably between 1° and 15° C., preferably between 15 and 25° C., preferably between 25 and 40° C., preferably between 4° and 100° C., preferably between 4° and 80° C., preferably between 5° and 75° C. at a pressure ranging between 1 and 100 bar, more preferably 1 and 70 bar. In a preferred embodiment the forming takes place at cold temperature or at room temperature, preferably at a temperature between 1 and 25° C., most preferably between 4 and 15° C. at a pressure ranging between 1 and 100 bar, more preferably 1 and 70 bar.

In one embodiment, the low-temperature extrusion is adopted, wherein the extrusion temperature preferably ranges between 3° and 100° C., more preferably between 4° and 80° C., more preferably between 5° and 75° C. at a pressure ranging between 5 and 25 bar.

In a second embodiment, the high-temperature extrusion is adopted, wherein the extrusion temperature preferably ranges between 100 and 190° C., more preferably between 12° and 180° C., more preferably between 13° and 180° C. at a pressure ranging between 100 and 300 bar, where in the moisture of the extrusion Is preferably below 45 wt. %, preferably below 40 wt. %, preferably below 35 wt. %.

In a third embodiment, the high-temperature extrusion is adopted, wherein the extrusion temperature preferably ranges between 10° and 190° C., more preferably between 12° and 180° C., more preferably between 13° and 180° C. at a pressure ranging between 100 and 300 bar, where in the moisture of the extrusion Is preferably above 40 wt. %, preferably above 45 wt. %, preferably above 50 wt. %.

In one embodiment, the rope of the extruded dough has a thickness ranging between 1 and 8 cm, preferably 1.5 to 5 cm, more preferably between 1.5 and 3.5 cm.

In a further embodiment, the product is frozen and stored at a temperature of −40° C., −30° C.,-20° C., −15° C., or −5° C., 0° C., 4° C., 5° C. In a preferred embodiment, the product is frozen at a temperature between −40 and 5° C., more preferably between −20 and 5° C., most preferably −5° C. and 5° C. In another preferred embodiment, the product is stored around −20° C. In another preferred embodiment, the product is stored around −4° C. In another embodiment, superchilling of food products is applied, wherein the product is stored at a temperature between −0.5 and −5° C.

In one embodiment, an edible meat substitute product comprises a mycelium ingredient ranging from 1 to 99 wt. %, wherein said mycelium ingredient is characterized by insoluble fiber content between 20 and 60 wt. % and an EUC between 30 and 200 g MSG/100 g.

In one embodiment, an edible meat substitute product comprises a mycelium ingredient ranging from 1 to 99 wt. %, wherein said mycelium ingredient is characterized by insoluble fiber content between 20 and 60 wt. % and an EUC of less than 200 g MSG/100 g.

In one embodiment, an edible meat substitute product comprises a mycelium ingredient ranging from 1 to 99 wt. %, wherein said mycelium ingredient is characterized by insoluble fiber content between 20 and 60 wt. % and an EUC between 200 and 500 g MSG/100 g.

In one embodiment, an edible meat substitute product comprises a mycelium ingredient ranging from 1 to 99 wt. %, wherein said mycelium ingredient is characterized by insoluble fiber content between 20 and 60 wt. % and an EUC of at least 30 g MSG/100 g.

In one embodiment, an edible meat substitute product comprises a mycelium ingredient ranging from 1 to 99 wt. %, wherein said mycelium ingredient is characterized by insoluble fiber content between 20 and 60 wt. % and an EUC of at least 500 g MSG/100 g.

In one embodiment, an edible meat substitute product comprises a mycelium ingredient ranging from 1 to 99 wt. %, wherein said mycelium ingredient is characterized by insoluble fiber content between 20 and 60 wt. % and an EUC of at least 1000 g MSG/100 g.

In one embodiment, an edible meat substitute product comprises a mycelium ingredient ranging from 1 to 99 wt. %, wherein said mycelium ingredient is characterized by insoluble fiber content between 20 and 60 wt. % and an EUC of at least 1500 g MSG/100 g.

In one embodiment, an edible meat substitute product comprises a mycelium ingredient ranging from 1 to 99 wt. %, wherein said mycelium ingredient is characterized by insoluble fiber content between 20 and 60 wt. % and an EUC of at least 2000 g MSG/100 g.

In one embodiment, an edible dairy substitute product comprises a mycelium ingredient ranging from 1 to 99 wt. %, wherein said mycelium ingredient is characterized by insoluble fiber content between 20 and 60 wt. % and an EUC between 30 and 200 g MSG/100 g.

In one embodiment, an edible dairy substitute product comprises a mycelium ingredient ranging from 1 to 99 wt. %, wherein said mycelium ingredient is characterized by insoluble fiber content between 20 and 60 wt. % and an EUC of less than 200 g MSG/100 g.

In one embodiment, an edible dairy substitute product comprises a mycelium ingredient ranging from 1 to 99 wt. %, wherein said mycelium ingredient is characterized by insoluble fiber content between 20 and 60 wt. % and an EUC between 200 and 500 g MSG/100 g.

In one embodiment, an edible dairy substitute product comprises a mycelium ingredient ranging from 1 to 99 wt. %, wherein said mycelium ingredient is characterized by insoluble fiber content between 20 and 60 wt. % and an EUC of at least 30 g MSG/100 g.

In one embodiment, an edible dairy substitute product comprises a mycelium ingredient ranging from 1 to 99 wt. %, wherein said mycelium ingredient is characterized by insoluble fiber content between 20 and 60 wt. % and an EUC of at least 500 g MSG/100 g.

In one embodiment, an edible dairy substitute product comprises a mycelium ingredient ranging from 1 to 99 wt. %, wherein said mycelium ingredient is characterized by insoluble fiber content between 20 and 60 wt. % and an EUC of at least 1000 g MSG/100 g.

In one embodiment, an edible dairy substitute product comprises a mycelium ingredient ranging from 1 to 99 wt. %, wherein said mycelium ingredient is characterized by insoluble fiber content between 20 and 60 wt. % and an EUC of at least 1500 g MSG/100 g.

In one embodiment, an edible dairy substitute product comprises a mycelium ingredient ranging from 1 to 99 wt. %, wherein said mycelium ingredient is characterized by insoluble fiber content between 20 and 60 wt. % and an EUC of at least 2000 g MSG/100 g.

In one preferred embodiment, the edible product preferably comprises a mycelium ingredient obtained from a Pleurotus spp. (Pleurotaceae or Pleurotus fungus), such as fungus selected from Pleurotus pulmonarius, Pleurotus ostreatus, Pleurotus citrinopileatus, Pleurotus florida, and Pleurotus salmoneostramineus, preferably Pleurotus pulmonarius; or a mycelium ingredient obtained from a fungus selected from Morchella esculenta, Morchella angusticeps, Morchella deliciosa, and Morchella rufobrunnea, and Morchella rufobrunnea.

In one preferred embodiment, the edible product comprises a mycelium ingredient obtained from Pleurotus pulmonarius. In another preferred embodiment, the edible product comprises a mycelium ingredient obtained from Morchella rufobrunnea. In a further embodiment, the edible product comprises a mycelium ingredient obtained from a combination of Pleurotus pulmonarius and Morchella rufobrunnea. In another embodiment, the edible product comprises a mycelium ingredient obtained from L. sulphureus. In another embodiment, the edible product comprises a mycelium ingredient obtained from B. adusta.

Meat and dairy analogues may be vegetarian or vegan. The above embodiments also apply for products that are not meat or dairy analogues, which may include, but are not limited to, fish substitutes, confectionery, baked products, flour (including bread or pasta or noodles), sweets and desserts, snacks, cereal products, alcoholic and non-alcoholic beverages, spice blends, instant meals, frozen meals, colloidal foods, protein supplements, and extruded/puffed products.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following examples are merely illustrative of the present invention and should not be construed to limit the scope of the invention which is defined by the appended claims in any way.

### EXAMPLES

For every constituent determined via wet-chemical analysis each sample is analyzed at least in duplicates, wherein the percentages are expressed on a dry basis. The embodied examples below were performed on the fungal strain from Pleurotus pulmonarius. It is to be understood that Pleurotus spp, in particular Pleurotus pulmonarius, is a preferred fungal strain in the methods and products of the present invention. However, the present invention is not limited to this strain, and can be practiced with other fungal strains as well.

### Example of Ingredient Production

As an example, medium A (defined medium) comprises the following composition 0.45 wt. % C, 0.026 wt. % N, 1.24 wt. % O, and 0.326 wt. % P to be used to produce ingredient A, where in the nitrogen source is CSL (10 g/l) and the carbon source is dextrose.

Medium B (synthetic medium) comprises the following composition: 0.41 wt. % C, 0.02 wt. % N, 1.2 wt. % O, and 0.326 wt. % P to be used to produce ingredient B, wherein the amino acid is selected from arginine (0.62 g/l) and the vitamins are sourced from D-Biotin 1 mg/L, Folic acid 1 mg/L, Niacinamide 1 mg/L, D-Pantothenic acid (hemicalcium) 1 mg/L, Pyridoxal HCl 1 mg/L, Riboflavin 0.1 mg/L, and Thiamine HCl 1 mg/L. The carbon source is dextrose.

Medium C (natural complex medium) comprises a spent grain extract extracted using steam pretreatment at 170° C. for a time less than 5 minutes, in particular for 3 minutes followed by nutrient recovery via washing in water, wherein the final protein content of the extract used in the fermentation is about 10 g/l with a total glutamate concentration of 566 mg/L and a total aspartate concentration of 216 mg/L Medium of C comprises of the following composition: 0.772 wt. % C, 0.059 wt. % N, 1.6 wt. % O, and 0.326 wt. % P. The spent grain had a particle size distribution, wherein 2-4 mm constituted the highest range of this distribution, about 35 wt. % of the total particle size distribution present. The carbon to nitrogen of the extract is about 11 (extract composition based on CHNO analysis: 1 wt. % C, 0.093 wt. % N, 0.81 wt. % O, and 0.12 wt. % H).

All mediums comprise the following salts based on potassium (0.1 g/l), sodium (2 g/l), iron(0.001 g/l), copper (0.01 g/l), magnesium (0.09 g/l), calcium (0.009 g/l), manganese(0.09 g/l), zinc (0.05 g/l).

The mycelium production is separated into two different lines. The seed line and the specific main fermentation in the respective media. The seed line is split into three distinct steps. First, mycelium is grown on a petri dish on a PDA medium. Then, it is cultured for 7 days in one of the media A or B or C for 7 days at 28° C. The whole culture is then cultivated at ambient temperature. For each respective main fermentation one culture from the seed line is used and cultivated aerobically in a fermentation system cultivated in a fermenter for about 5 to 6 days until complete carbon source consumption. In all three cases (A, B and C) the fermentation is done aerobically at ambient temperatures (25° C.) and at a pH ranging from 4 to 5. In these examples, the pH of the fermentation medium was about 5. In fermentation, it is also usual to control dissolved oxygen. The ingredients are then harvested and ready to be used for further processing after washing them with acidic water at a pH 3.5. It is to be understood, unless explicitly indicated to the contrary, that the medium composition (e.g. specific compositions of media A, B and C) as used herein, corresponds to the media compositions at the beginning of the fermentation. It is apparent to the skilled person, as indicated hereinabove, that within time the composition of the medium may change and e.g. certain components, for example carbon source, may be at least in part consumed.

### Freeze-Drying Method of Ingredients a, B, and C

The freeze drying is performed in a “Christ Alpha 1-2” freeze dryer connected to an external vacuum pump, where in the respective sample (A, B, or C,) is freeze dried at −50° C. with a vacuum of 6 mbar directly applied. All samples were pre-frozen at −80° C. The samples were freeze-dried for a duration of 72 hours.

### BET Surface by Gas Adsorption (According to DIN-ISO 9277, Respectively DIN 66131)

During the gas adsorption (according to DIN-ISO 9277 respectively DIN 66131) the specific surface of solid matters is determined by default with nitrogen adsorption at 77.4 K using the BET method. The sample preparation takes place at the individual specified temperatures. The sample surface must not alter in the meantime. At the statistic-volumetric method a specific amount of measuring gas is dosed onto the sample which is arranged in vacuum. The determination of the adsorbed gas quantity is based on a gas equation and a pressure measurement in volume calibrated systems. At the dynamic method the determination of the adsorbed gas quantity is carried out by a thermal conductivity sensor which detects the change of the gas composition in a N2/He-mixture. From the measurement readings (adsorbed volume Va vs. relative pressure p/p0) the calculation of the molecule amount in a mono layer on the solid matter surface is carried out. The evaluation takes place in the general area of validity of the BET method of p/p0=0.05−0.3 respectively in the stated relative pressure area. For the determination of very small surfaces the krypton adsorption (at 77.4 K) is used. Because the expected surface areas are quite low, sample cells have been filled completely with sample (approx. 0.5-0.6 g per sample-see attached data sheets) and Krypton has been used, because Krypton is the suitable adsorbate for measuring low surface areas. Before analysis samples have been degassed at room temperature under vacuum for approx. 65 hours. The analysis has been performed on a Quantachrome Quadrasorb.

### Mercury Porosimetry (According to DIN-ISO 15901-1, Respectively DIN 66133)

Pore analysis by mercury porosimetry is performed with a Quantachrome Poremaster-60GT. Method is based on the Washburn-Equation, which describes the relationship between pore diameters and applied pressure to a non-wetting liquid like mercury. Using the Poremaster-60GT, filling of the penetrometers before analysis is performed in horizontal position: due to this fact a hydrostatic mercury pressure on the sample is also avoided as a non-detected pore filling. The resulting intrusion curves will be plotted over pressure, resp. pore diameter. Because the measurement starts at low pressures, large pores will be filled first and therefore-in standard cases-pore size decreases from left to right side of the chart. The upper method limit is approx. at 1 mm pore diameter, that means that the detection of larger pores was not the goal of this experiment.

### Free Bases, Nucleosides and 5′NMP Nucleotides

The freeze-dried ingredient is dissolved in water 25 mg/mL, heated at 100° C. for 10 min and homogenized. For protein precipitation, TCA was added to a 5% final concentration, incubated at −20° C. for 16 h. 5 L of deproteinized extract was then injected and analyzed by ion-paired HPLC.

### RNA Quantification Method

To quantify RNA, nuclease was added to solubilized sample (25 mg/mL) and incubated for 16 h at 60° C. to convert RNA to 5′NMP For protein precipitation, TCA was added to a 5% final concentration, incubated at −20° C. for 16 h. 5 μL of deproteinized extract was then injected and analyzed by ion-paired HPLC.

The flavors in terms of the known flavor 5′ nucleotides (5′NMP) in g/kg, which include 5′-inosine monophosphate (IMP), 5′-guanosine monophosphate (GMP) and 5′-adenosine monophosphate (AMP) were measured. Ingredient A shows a good richness of 5′NMP of 3.56 g/kg compared to B showing 1.78 g/kg. However, the usage of spent grain extract led to 10-fold increase in ingredient C compared to C, leading to a 5′NMP of 10.43 g/kg. Additionally, ingredient C showed the highest concentration of Uridine monophosphate (UMP) of 3.56 g/kg compared to 1.26 g/kg and 0.71 g/kg for A and B, respectively.

Without any extra ribonucleic acid (RNA) reduction step in the production process of the ingredients via additional heating or pH treatment, the method of this invention leads to an inherent RNA level of the ingredients below 2 wt. %, specifically 1.88 wt. % for A, 1.65 wt. % B and 2 wt. % for C in one embodied example.

TABLE 1
5′-nucleotides and RNA content of
the mycelium ingredients A, B, and C

| | | A | B | C |
|---|---|---|---|---|
| Bases | Cytosine, g/kg | 0.06 | 0.10 | 0.39 |
| | Uracyl, g/kg | 0.19 | 0.13 | 0.22 |
| | Guanine, g/kg | 0.07 | 0.07 | 0.13 |
| | Hypoxanthine, | 0.02 | 0.01 | 0.03 |
| | g/kg | | | |
| | Adenine, g/kg | 0.28 | 0.17 | 0.54 |
| Nucleosides | Cytidine, g/kg | 0.19 | 0.16 | 1.14 |
| | Uridine, g/kg | 0.99 | 0.49 | 1.12 |
| | Guanosine, g/kg | 1.46 | 0.52 | 1.41 |
| | Inosine, g/kg | 0.00 | 0.00 | 0.00 |
| | Adenosine, g/kg | 1.31 | 0.59 | 0.83 |
| NMP | CMP, g/kg | 0.18 | 0.10 | 0.34 |
| | UMP, g/kg | 1.26 | 0.71 | 3.56 |
| | GMP, g/kg | 1.45 | 0.77 | 5.85 |
| | IMP, g/kg | 0.00 | 0.00 | 0.00 |
| | AMP, g/kg | 2.01 | 1.01 | 4.58 |
| RNA | CMP, g/kg | 4.87 | 3.15 | 5.15 |
| | UMP, g/kg | 5.34 | 3.89 | 4.63 |
| | GMP, g/kg | 2.27 | 3.12 | 4.23 |
| | IMP, g/kg | 0.00 | 0.00 | 0.00 |
| | AMP, g/kg | 4.40 | 3.89 | 5.24 |
| Free bases, g/kg | Sum of the free | 0.62 | 0.48 | 1.30 |
| | bases | | | |
| Free purine | Sum of Guanosine, | 2.77 | 1.11 | 2.24 |
| nucleosides, | Inosine, and | | | |
| g/kg | Adenosine | | | |
| Flavor 5′ | Sum of IMP, GMP, | 3.46 | 1.78 | 10.43 |
| nucleotides, | and AMP | | | |
| g/kg | | | | |
| RNA, wt. % | Sum of CMP, UMP, | 1.88 | 1.65 | 2 |
| (dry basis) | GMP, IMP, and | | | |
| | AMP | | | |

### Amino Acids Quantification Method and Data

The amino acid analysis is by acid hydrolysis of samples followed by HPAEC-IPAD. Tryptophan is not detected with this method as tryptophan undergoes degradation during acid hydrolysis of samples.

All ingredients A, B, and C show an umami amino acid of at least about 20 wt. %, more specifically, 21.48 wt. % for A, 19.5 wt. % for B, and 22.64 wt. % for C.

All ingredients A, B, and C show a BCAA content of at least about 20 wt. %, more specifically, 21.73 wt. % for A, 23.1 wt. % for B, and 19.6 wt. % for C.

All ingredients A, B, and C show content of the essential amino acids of at least about 40 wt. %, more specifically, 40.81 wt. % for A, 40.52 wt. % for B, and 39.61 wt. % for C.

The total amino acid content in mg/g is richest in C (361.67 mg/g), followed by A (268.85 mg/g), followed by C (163.43 mg/g).

TABLE 2
Amino acid content of the mycelium ingredients A, B, and C

| Amino Acids | A | A (% of | B | B (% of | C | C (% of |
|---|---|---|---|---|---|---|
| (AA) | (mg/g) | total AA) | (mg/g) | total AA) | (mg/g) | total AA) |
| Aspartic Acid | 26.42 | 9.83 | 14.95 | 9.15 | 23.23 | 6.42 |
| Glutamic Acid | 31.34 | 11.66 | 16.90 | 10.35 | 58.66 | 16.22 |
| Umami AA | | 21.48 | | 19.50 | | 22.64 |
| Leucine | 24.42 | 9.08 | 16.24 | 9.94 | 32.19 | 8.90 |
| Isoleucine | 16.81 | 6.25 | 10.49 | 6.42 | 19.59 | 5.42 |
| Valine | 17.18 | 6.39 | 11.00 | 6.73 | 19.12 | 5.29 |
| BCAAs | | 21.73 | | 23.10 | | 19.60 |
| Methionine | 0.00 | 0.00 | 0.00 | 0.00 | 4.89 | 1.35 |
| Phenylalanine | 17.76 | 6.60 | 8.83 | 5.41 | 15.48 | 4.28 |
| Threonine | 13.27 | 4.94 | 9.56 | 5.85 | 18.68 | 5.16 |
| Histidine | 7.64 | 2.84 | 3.76 | 2.30 | 12.60 | 3.49 |
| Lysine | 12.64 | 4.70 | 6.30 | 3.86 | 20.70 | 5.72 |
| EAAs incl. | | 40.81 | | 40.52 | | 39.61 |
| BCAs | | | | | | |
| Arginine | 19.98 | 7.43 | 14.53 | 8.89 | 29.67 | 8.20 |
| Glycine | 17.62 | 6.55 | 10.75 | 6.58 | 22.98 | 6.35 |
| Serine | 15.88 | 5.90 | 10.23 | 6.26 | 23.57 | 6.52 |
| Cystine | 0.00 | 0.00 | 0.07 | 0.04 | 0.46 | 0.13 |
| Proline | 14.81 | 5.51 | 9.08 | 5.56 | 18.75 | 5.19 |
| Alanine | 26.71 | 9.93 | 15.58 | 9.54 | 30.09 | 8.32 |
| Tyrosine | 6.38 | 2.37 | 5.08 | 3.11 | 11.01 | 3.04 |
| Total AA | 268.85 | | 163.34 | | 361.67 | |

### Biomass Characterization and EUC Concentration

The mycelium ingredient A is grown on a medium containing a carbon to nitrogen ratio (medium) of about 17. The mycelium ingredient B is grown on a medium containing a carbon to nitrogen ratio (medium) of about 20. Mycelium ingredient B is grown on a medium containing a carbon to nitrogen ratio (medium) of about 13.

The insoluble fiber content increases from C to A to B from around 23 wt. %, to around 34 wt. % to around 54 wt. %, while the protein content from C to A to B decreases from around 60 wt. % to around 39 wt. % to around 32 wt. %. This shows the relationship discussed earlier between proteins and fibers, that they are inversely proportional and can be controlled by varying the fermentation medium composition.

The fat content of ingredient A is about 3 wt. %. The fat content of ingredient A is about 2 wt. % The fat content of ingredient C is about 7 wt. %. This fat comprises of saturated fatty acids, monosaturated fatty acids, polyunsaturated fatty acids and trans fatty acids, as shown in the table below. The mushroom mycelium is highest in saturated fatty acids (up to 1.5 g/100 g for A and B, up to 2 for C), whereas C is rich in Polyunsaturated fatty acids (3.63 g/100 g). The ingredients contain minimal amounts of trans fatty acids (up to 0.02-0.03 g/100 g). It is worth noting that the content of omega-6 fatty acid (linoleic acid) is 0.77 wt. for A, 0.46 wt. % for B and 3.5 wt. % for C. All the ingredients show a calculated carbohydrate content of less than 0.1 wt. %. B is the richest in glucans total, with a value of 31 wt. %. The mycelium ingredient A has a total glucans value of about 26 wt. %. The mycelium ingredient C has a total glucans value of about 17 wt. %.

TABLE 3
Compositional and nutritional profile
of the mycelium ingredients A, B, and C

| | A | B | C |
|---|---|---|---|
| C:N ratio of fermentation medium | 17.44 | 20.32 | 13 |
| C:N ratio of mycelium | 7.11 | 9.5 | 4.95 |
| Protein (g/100 g, dry weight) | 38.81 | 32.53 | 60.66 |
| Fat (g/100 g, dry weight) | 2.91 | 2.15 | 6.75 |
| Ash (g/100 g, dry weight) | 9.50 | 9.70 | 8.40 |
| Soluble dietary fiber (g/100 g, dry) | 5.89 | 5.42 | 2.89 |
| Insoluble dietary fiber (g/100 g, dry) | 34.34 | 53.54 | 23.37 |
| Total dietary fiber (g/100 g, dry) | 40.23 | 58.96 | 26.26 |
| Carbohydrates (g/100 g, dry) | <0.1 | <0.1 | <0.1 |
| Energy value (Kcal/100 g, dry weight) | 425 | 435 | 455 |
| Saturated fatty acids (g/100 g, dry) | 1.52 | 1.06 | 1.96 |
| Monounsaturated fatty acids (g/100 g, dry) | 0.59 | 0.59 | 1.11 |
| Polyunsaturated fatty acids (g/100 g, dry) | 0.78 | 0.47 | 3.63 |
| Trans fatty acids (g/100 g, dry) | 0.02 | 0.02 | 0.03 |
| Palmitic acid (C16:9) (g/100 g, dry) | 0.70 | 0.53 | 1.45 |
| Stearic acid (C18:0) (g/100 g, dry) | 0.40 | 0.28 | 0.28 |
| Lignoceric acid (C24:0) (g/100 g, dry) | 0.13 | 0.08 | 0.06 |
| Oleic acid (C18:1w9c) (g/100 g, dry) | 0.48 | 0.54 | 0.96 |
| Linoleic acid (C18:2w6c) (g/100 g, dry) | 0.77 | 0.46 | 3.50 |
| Ergosterol (mg/g, dry weight) | 2.35 | 2.24 | 5.56 |
| Ergothioneine (mg/kg, dry weight) | 91 | 127 | 455 |
| Chitin (wt. %) | 7.6 | 6.9 | 7.5 |
| Total Glucan (wt. %) | 25.6 | 31.2 | 16.8 |
| Beta-glucan (wt. %) of total glucan | 81 | 84.8 | 87.2 |
| Alpha-glucan (wt. %) of total glucan | 19 | 15.2 | 12.8 |
| TPC (mg GAE/g) | 3.2 | 2.7 | 4.5 |
| TFC (mg QE/g) | 1.7 | 2.5 | 3 |
| EC50 (mg/ml) | 10.5 | 13 | 9 |
| Polyphenols (mg/kg, dry weight) | 167 | 74 | 630 |
| Catechin (mg/kg, dry weight) | 70 | 22 | 317 |
| Protocatechuic acid (mg/kg, dry weight) | 72 | 32 | 310 |
| Quercetin (mg/kg, dry weight) | 8 | 12 | 0 |
| Free Aspartic Acid (mg/g) | 5.995 | 1.305 | 15.392 |
| Free Glutamic acid (mg/g) | 0.574 | 0.090 | 0.813 |
| 5′-AMP (g/kg) | 2.216 | 1.095 | 4.925 |
| 5′-GMP (g/kg) | 1.607 | 0.843 | 6.29 |
| 5′-IMP (g/kg) | 0 | 0 | 0 |
| EUC (g MSG/100 g dry weight) | 302 | 34 | 2892 |

It was calculated that the EUC for the mycelium ingredients is 302% for A, 34% for B, and 2892% for C.

In a separate study, P. pulmonarius fruiting bodies were grown on three forestry wastes (pine, poplar, and honeysuckle rattan), showing EUC values of P. pulmonarius fruiting bodies between 72.31% and 116.73% (Food Chemistry 397 (2022) 133714). The disclosed EUC values of mycelium ingredients A, B, and C of this invention are higher than this reported value for a fruiting body of the same fungal strain, and taking ingredient C as an example, the difference is 25 to 40-fold higher for ingredient C compared to the reported range of P. pulmonarius fruiting bodies. Knowing also that the highest value of EUC of a mushroom fruiting body was reported to be 4465% for (Volvariella volvacea), which is lower by a factor of 1.54 compared to ingredient C.

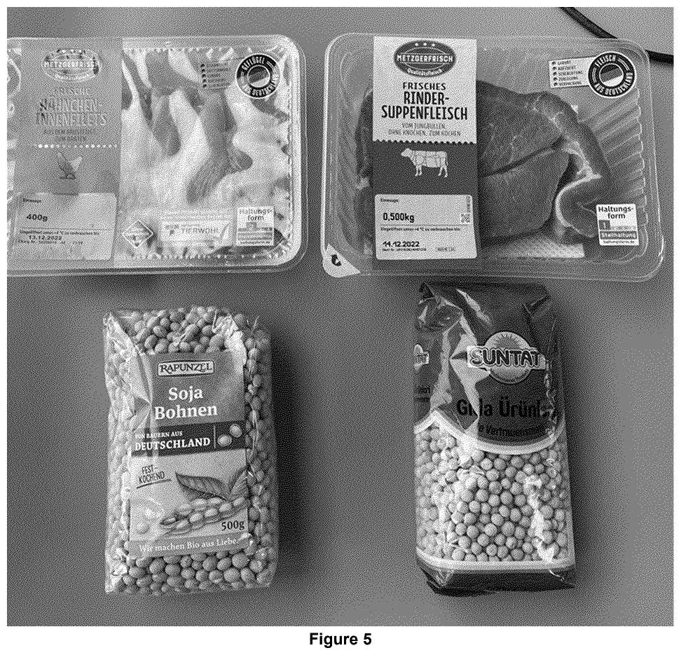

Furthermore, the EUC concentration of beef, chicken, green peas and soya beans (see FIG. 5 for the exact products that were used in the comparison) were measured to compare them with the mycelium ingredients A, B, C, where the found EUC were found to be, 78.77%, 134.7%, 65%, and 1% respectively.

### Total Sugar Quantification Method

Approximately 300 mg of the ingredient is added to a pressure tube. 3 mL of 72% H2SO4 is added by means of an automatic titrator, the weight of the acid added is noted. The sample is mixed thoroughly with the acid using a glass rod. The tube is transferred to a water bath that is maintained at 30° C. The steps are repeated for the duplicate sample. Every 10 minutes the glass rod for each pressure tube is stirred so that the acid reaches all parts of the sample and complete hydrolysis occurs. Exactly one hour after it is placed in the water bath the pressure tube is removed and placed on a scales and 84 mL of water added (with the weight of the added water recorded). Any acid/sample on the rod is removed from the rod at this point using this water. A lid is screwed on the tube and the tube is inverted several times to ensure thorough mixing of the acid. Two sugar recovery solution (SRS) pressure tubes are prepared to monitor the sugar-loss associated with the second stage-hydrolysis. This involves the following steps: (a) 348 microliters of 72% H2SO4 is added to a test tube containing a solution containing a known weight (approximately 10 g) of a sugar standard. This standard should be of a similar sugar composition to that expected of the samples being analyzed. The acid and sugar solution are thoroughly mixed. (b) The sugar-acid mixture is transferred to a pressure tube which is then sealed. All SRS and sample pressure tubes are placed in an autoclave which is run at 121 degrees Celsius for 60 minutes. After the temperature in the autoclave drops to under 80° C., he tubes are removed and are left (closed) in the lab until they reach room temperature. The hydrolysates are then filtered (using vacuum suction) through filter crucibles of known weight and the resulting filtrate is stored. Any residual solids are washed out from the tube using deionized water until all the acid insoluble residue resides on the filter crucible. Following the Acid Hydrolysis Step, the hydrolysate (the filtrate from the vacuum filtration) is diluted by a factor of 5 using a fucose-in-water solution. Fucose is the internal standard that is used in the chromatographic analysis of the hydrolysate. Following this dilution, the samples are immediately put on the chromatography system. Equipment used are NIR Spectrophotometers (FOSS XDS NIR) and Ion Chromatography (ICS-3000).

### Sugars in Water Extract Data

The ingredients were hydrolysed using acid to sugars (glucan, xylan, arabinan, galactan, mannan, rhamnan) contents. However, this hydrolysis was carried out on the original sample with no extractives (e.g., ethanol or water-soluble components) removed. All data is expressed as a percentage of the total dry mass.

TABLE 4
sugar profiles of the mycelium ingredients A, B, and C

| | Total | Glucan | Xylan | Arabinan | Galactan | Mannan | Uronic | | |
|---|---|---|---|---|---|---|---|---|---|
| Ingredient | Sugars | (Hexosans) | (pentosans) | (Pentosans) | (Hexosans) | (Hexosans) | Acids | Ash | Extractives |
| A | 39.4 | 34.7 | 0.56 | 0 | 0.75 | 1.8 | 1.58 | 9.5 | 32.5 |
| B | 54.9 | 50.78 | 0.46 | 0 | 0.67 | 1.56 | 1.44 | 9.7 | 24.7 |
| C | 26.75 | 21 | 0.9 | 0.42 | 0.85 | 1.56 | 2 | 8.4 | 30 |

The most abundant sugar as a percentage of the total sugar content in A is 88% for glucan, 92.5% in B for glucan, 78.6% in C for glucan (the glucan here is based on all the glucose, including glucose derived from other polysaccharides, such as heteroglycan and/or exopolysaccharide).

Sugars in the water extract or extractives shown in the above table, which comprises the sugars extracted via water from fresh mycelium ingredients. The water extraction was done with ASE 200 (Accelerated Solvent Extractor) in 11 ml stainless steel extraction cells, wherein deionized water at 100° C., 1500 psi was used (heat time 5 min, static time: 7 min, flush volume: 150%, purge time: 180 sec, static cycles: 3). The total sugars in the water extracts of ingredient A, B, and C are 6.25%, 7%, and 3.26% respectively, from which, for A, the sugars in the water extract expressed as the % of the total extract sugars comprises of disaccharides (trehalose 20.1%), hexose sugars (glucose 22%, fructose 0.5%, mannose, 0.1%, galactose 1.4%), pentose sugars (arabinose 0.1%) and sugar alcohols (mannitol 55%, sorbitol 0.7%). For B, the sugars in the water extract expressed as the % of the total extract sugars comprises of disaccharides (trehalose 19.2%), hexose sugars (glucose 43.3%, fructose 0.4%, galactose 0.1%), pentose sugars (arabinose 0.1%) and sugar alcohols (mannitol 35.7%, sorbitol 1%). For C, the sugars in the water extract expressed as the % of the total extract sugars comprises of disaccharides (trehalose 46%), hexose sugars (glucose 16%, galactose 0.1%), pentose sugars (arabinose 0.03% and xylose 0.15%) and sugar alcohols (mannitol 22.7%, arabinitol 13.5%, xylitol 1%, sorbitol 0.64%).

### Elemental Analysis, Ash Content and Gross Calorific Value Determination Methods

The ultimate (elemental) analysis of biomass will provide the mass concentrations of the major elements (carbon, oxygen, hydrogen, nitrogen, and sulfur) in the samples. The carbon, hydrogen, nitrogen, and sulfur contents of samples were measured according to the procedures outlined in European Standard EN 15104:2011 (“Solid biofuels-Determination of total content of carbon, hydrogen and nitrogen-Instrumental methods”). An Elementar Vario MACRO Cube elemental analyzer was used. The oxygen content by difference according to the formula below: Oxygen (%)=100−Carbon (% Dry Basis)−Hydrogen (% Dry Basis)−Nitrogen (% Dry Basis)−Sulphur (% Dry Basis)−Ash (% Dry Basis). To carry out this calculation the ash content of the sample is also measured in a Nabertherm L-240H1SN muffle furnace and heated up to 575 C. The as samples are then weighed to calculate the ash content. The Higher Heating Value (HHV, often referred to as the Gross Calorific Value) was determined directly using an oxygen bomb calorimeter as outlined in EN 14918:2009. The Lower Heating Value (LHV, often referred to as the Net Calorific Value) was calculated based on the HHV and the elemental composition of the sample.

### Elemental Analysis, Ash Content and Gross Calorific Value Data

The sample was also analyzed for its ash content and its elemental composition (carbon C, hydrogen H, nitrogen N, sulfur S, oxygen O) with the oxygen content determined by difference. The ash content of ingredients A, B, and C are 9.39%, 9.73%, and 8.40%.

The elemental analysis of C, H, N, O, S analysis of these ingredients leads to,

For A: 44.45% (Carbon C), 5.95% (H), 6.25% (N), 0.32% (S), 33.53% (O), 0.0815% (chlorine); Carbon to nitrogen ratio: 7.11.

For B: 43.92% (Carbon C), 5.83% (H), 4.62% (N), 0.26% (S), 35.64% (O), 0.0788% (chlorine); Carbon to nitrogen ratio: 9.5.

For C: 46.90% (Carbon C), 6.26% (H), 9.57% (N), 0.42% (S), 28.45% (O), 0.0703% (chlorine); Carbon to nitrogen ratio: 4.9.

The lower nitrogen content in sample B indicates a sample richer in fiber content but having a lower protein content, whereas sample C, based on the brewery spent grain, has the highest nitrogen content indicating a high protein content with a lower fiber content. Sample A comes in between the two. This indicates that the protein and fiber content can be tweaked by tweaking the composition of the medium either synthetically or by using nutritious side stream with known elemental compositions, allowing the production of tailored food products, either richer in fiber or in protein.

The Higher Heating Value (HHV, often referred to as the Gross Calorific Value) was determined directly using an oxygen bomb calorimeter as outlined in EN 14918:2009. The Lower Heating Value (LHV, often referred to as the Net Calorific Value) was calculated based on the HHV and the elemental composition of the sample. For A is 18.4 MJ/kg and 17.11 MJ/kg; For B 18.88 MJ/kg and 17.61 MJ/kg; For C 19.73 MJ/kg and 18.37 MJ/kg

### Other Determination Methods

### Chitin Analysis

For the determination of chitin content, the method in the following paper is used (doi.org/10.1155/2020/5084036). A maximum recovery of glucosamine was ensured. The glucosamine content was estimated by ion chromatography. Chitosan was used as the experimental control.

### Ergothioneine Analysis

For the quantification of ergothioneine, approximately 1.25 g of sample was weighed into 25 mL volumetric flasks. The volumetric flask was filled to the mark with cold 70% ethanol to which 10 mM dithiothreitol was added and sonicated for 15 minutes. The samples were then centrifuged at 4000 rpm for 20 min to separate the insoluble material. 2 mL of the supernatant obtained was evaporated to dryness. The residue was re-suspended in 1 ml of water and centrifuged at 10,000 rpm for 1 minute. Finally, the supernatant was filtered through a 0.45-μm filter and further diluted with water before HPLC injection.

HPLC analyses of ergothioneine were performed on a Prominence system (Shimadzu, Duisburg, Germany) equipped with an LC-20AD high-performance liquid chromatography (HPLC) pump, SIL-20AC HT autosampler, SPD-M20A diode array detector (DAD), CBM-20A communication bus module, and LabSolutions Multi LC Data System Manager. An EC 250/4 Nucleosil 100-5 C18 column (Macherey-Nagel, Düren, Germany) was used with a matching precolumn. The injection volume was 10 μL. The mobile phase used was 3% acetonitrile and 0.1% acetic acid in water. Separation was performed under isocratic elution at a flow rate of 0.7 mL/min and ambient temperature for 15 min, and detection was performed at 254 nm. External calibration with ergothioneine dissolved in water (5 μg·mL-1 to 100 μg·mL-1) was used for quantification.

### Ergosterol Analysis

Ergosterol was quantified by internal calibration using 7-dehydrocholesterol as an internal standard. For ergosterol analysis, 150-200 mg of each sample was weighed into a derivatization tube and mixed with 50 mg sodium ascorbate and 250 μL 7-dehydrocholesterol stock solution (5 mg mL-1 in 2-butanone). After the addition of 5 mL of methanolic sodium hydroxide solution and homogenization by vortex, the tubes were incubated for a total of one hour at 80° C. in a water bath. Shaking was performed after every 20 min. After the samples were cooled to room temperature in the dark, each sample was membrane filtered and transferred to a new derivatization tube. Subsequently, three extractions with hexane were performed. The hexane phases were combined in a 15 mL volumetric flask and the flask was filled ad mark with hexane. After drying with sodium sulfate, 6 mL was transferred to a new derivatization tube. The solvent was then removed under a stream of nitrogen. The residue was taken up in 0.5 ml of tetrahydrofuran (THF) and 0.5 mL of N-methyl-N-trimethylsilyltrifluoroacetamide (MSTFA) and dissolved in an ultrasonic bath. The solution was then heated at 70° C. in a water bath for 2 min. After one minute each, homogenization was performed by vortex. Silylation was carried out overnight. After transferring to vials, the samples were analyzed by gas chromatography (GC) and flame ionization detector (FID). A calibration series with 1 mL each of the 7-dehydrocholesterol stock and 0.25 to 4.5 mL ergosterol stock (2 mg·mL-1 in 2-butanone) was prepared in 10 mL volumetric flasks. 2-Butanone was used to make up ad mark, and 1 mL was transferred to a derivatization tube and dried under a stream of nitrogen. Subsequently, the calibration series were recorded and silylated in THF and MSTFA analogously to the samples. An ergosterol content of 2.35 g/g dry weight was determined for sample A, 2.24 g/g dry weight for sample B, and 5.56 g/g dry weight for sample C.

### Further Analyses

The protein content was determined according to DIN EN ISO 16634-1, 2009-07, (N*6.25) DUMAS. The fat content was determined according to Weibull-Stoldt. The fiber content was determined according to enzymatic-gravimetric method ASU L 00.00-18. The carbohydrate content was determined via HPLC according to SOP M 2569.

The contents of vitamins were determined by an A Dionex ICS-3000 ion chromatography system that is equipped with electrochemical, conductivity, and ultraviolet-visible detectors. Megazyme's Beta glucan assay kit for yeast and mushroom was used for alpha/beta glucan determination. The thermogravimentric analysis (TGA) was performed in a TA Instruments Q500 TGA unit that allows monitoring the weight loss of samples up to 1000 C under nitrogen atmosphere (ramp used: equilibrate at 35 C, isothermal for 2 min, equilibrate at 105 C, Isothermal for 5 min, Ramp 20C/min to 900 C).

Brands of goods purchased for EUC calculations: Beef and chicken (METZGERFRISCH, Rinder-SupppenFleisch and Hänschen-Innenfilets, respectively), green peas (Sunat) and soja beans (Rapunzel).

### Density Measurement

The density of mycelium ingredient samples was measured according to the Archimedean principle using an analytical density balance, KERN EMB-V (KERN & SOHN GmbH, Balingen, Germany) and YDB-01 Set (KERN & SOHN GmbH, Balingen, Germany). The tests were done at room temperature (=21° C.) and relative humidity of 50-60% and performed in triplicate. The kit provided a weighing plate, platform, beaker, and immersion baskets for descending and floating solid matters. The samples were weighed in air and distilled water (or other suitable liquids for densities below 1 g/cm³), and the density was directly calculated in g/cm3.

In one example, the ingredient density was found to be 1.012 g/cm³. The density of the mycelium ingredients ranges between 0.1 to 2.5 g/cm³, most preferably between 0.7 and 1.2 g/cm³.

TABLE 5
Density of a mycelium ingredient

| | Density |
|---|---|
| Test ID | (g/cm³) |
| Sample_1 | 1.0100 |
| Sample_2 | 1.0090 |
| Sample_3 | 1.0132 |
| Average | 1.0107 |
| S.D. | 0.0022 |

(S.D. = standard deviation)

### Shear Force Methodology

Shear forces were produced using a TA.XTplus100 Texture Analyzer (Stable Micro Systems, Surrey, United Kingdom). 50 kg and 100 kg load cells were used for the tests. Samples were analyzed at ambient temperature (˜ 21° C.). Due to the non-uniformity of the biomass, 5 g of sample was sheared, compressed, and extruded in bulk using a Miniature Kramer Shear/Ottowa Cell (HDP/MKS5). The results were given as an average of the forces (N) required to cut through the sample. In addition to the force, the areas under the curve (i.e., work of shear) were calculated (N·s) as well.

TABLE 6
shear force of a mycelium ingredient

| | Max | Area |
|---|---|---|
| Test ID | Force (N) | (N · s) |
| Sample 1 | 43.019 | 281.352 |
| Sample 2 | 44.097 | 300.464 |
| Sample 3 | 40.733 | 284.220 |
| Average | 42.616 | 288.679 |
| S.D. | 1.718 | 10.307 |
| Coef. of | 4.031 | 3.570 |
| Variation | | |

### Water Holding Capacity (WHC) and Release Water (RW)

Water holding capacity (WHC) describes the ability of the material to retain the water during processing. Among many procedures for measuring WHC, in the present study, it was determined using methods modified from van der Sman et al. and Liu et al. (Van der Sman, R. G. M.; Paudel, E.; Voda, A.; Khalloufi, S. Hydration properties of vegetable foods explained by Flory-Rehner theory. Food Research International 2013, 54, 804-811 and Liu, C.; Li, W.; Lin, B.; Yi, S.; Ye, B.; Mi, H.; Li, J.; Wang, J.; Li, X. Comprehensive analysis of ozone water rinsing on the water-holding capacity of grass carp surimi gel. Food Science and Technology 2021, 150, 111919.)

After separation of biomass and supernatant, 3 g of wet biomass were placed into a 50 ml falcon tube consisting of a ball of cotton and filter paper layers at the bottom. The samples were centrifuged at 4500 rpm for 10 minutes at 23° C. During centrifugation, the water was removed from the mycelium particles through the filter and was collected in the lower section. After centrifugation, the biomass was carefully removed and weighed. The falcon tube with the cotton and filter papers were also weighed for the comparison of the results. The WHC (%) was calculated according to the following equation:

$$W H C ( \% ) = \frac{Weight of solid sample after centrifugation ( g )}{Initial weight of biomass ( g )} \times 100$$

Released water was determined in triplicates according to the study of Seon-Tea Joo (Joo, S. Determination of water-holding capacity of porcine musculature based on released water method using optimal load. Korean journal for food science of animal resources 2018, 38(4), 823.). Approximately 1.0 g of drained biomass was weighed and carefully placed on previously desiccated and weighed two thin plastic films (PP5) and filter paper (MN 615, 12.5 cm in diameter). 100 g weight was applied for 5 min using metal circular plates as weights. After accurately removing the compressed biomass sample, damp filter paper and two plastic films were weighed. The RW (%) was defined according to the equation below:

$$R W ( \% ) = \frac{( Weight of damp filter paper and plastic films , g ) - ( Weight of dry filter paper and plastic films , g )}{Initial weight of biomass ( g )} \times 100$$

TABLE 7
WHC and RW of a mycelium ingredient

| | WHC | RW |
|---|---|---|
| Test ID | (%) | (%) |
| Sample 1 | 42.00 | 49.00 |
| Sample 2 | 52.32 | 43.73 |
| Average | 47.16 | 46.37 |
| S.D. | 7.30 | 3.73 |

### Puncture Test and Spreadability of a Dairy Analogue (Cream Cheese)

### Puncture Test

Measurements were taken with a TA.XTplus 100 Texture Analyzer equipped with a 5 kg load cell (Stable Micro Systems, Surrey, United Kingdom). Samples were prepared in polypropylene plastic boxes (inside diameter=7.8 cm, height=4.4 cm) and subjected to a puncture test using a cylindrical perspex probe (P/20P) with diameter of 20 mm. The probe punctured the cheese samples to a depth of 20 mm at a test speed 1 mm·s-1, pre test speed of 1 mm·s-1 and post test speed of 10 mm·s-1. Samples were analyzed at 16° C. to minimize differences in the solid fat contents. Three measurements were taken for each of the three replicates. The deformation level was appropriately selected to ensure complete penetration of the probe through the co-extrudate. Force (N) versus time(s) data were recorded and some mechanical parameters were determined using Exponent software (Stable Micro Systems, Surrey, UK). Maximum positive force (N), maximum negative force (N), Positive area (N·s), Negative area (N·s) parameters were determined. The maximum force is highly correlated with hardness of the cheese samples (Journal of Texture Studies, 32:41-55).

### Cream Cheese Spreadability

Spreadability tests were performed using a TA.XTplus100 Texture Analyzer (Stable Micro Systems, Surrey, United Kingdom). A 5 kg load cell was used for the test. Samples were analyzed at 16° C. to minimize differences in the solid fat contents. The TTC spreadability rig (HDP/SR) comprised a male 90° cone probe and five precisely matched female perspex cone shaped product holders. Cream cheese samples were filled with a spatula and then the surfaces were leveled. The sample holders were stored at the temperature stated above before testing the sample. Degree of spreadability was calculated during the tests when the product is forced to flow outward at 45° between the male and female cone surfaces. Force-time curves were recorded (Figure below), with the force (N) at the maximum penetration depth being taken as sample firmness. The area under the positive curve (N·s) represented the total amount of force required to perform the shearing process, and it has been considered a good instrumental measurement of spreadability in cream cheeses and in other spreadable products. Smaller values in this area indicate easier spreadability. The force (N) of the maximum negative peak indicates sample stickiness, and the maximum negative area (N·s) represents the work of adhesion (Bayarri, S.; Carbonell, I.; Costell, E. Viscoelasticity and texture of spreadable cheeses with different fat contents at refrigeration and room temperatures. Journal of Dairy Science 2012, 95(12), pp. 6926-6936.).

### Comparison Between Two Mycelium-Based Cream Cheeses

Sample 1 is an example of a dairy analogue comprising the mycelium ingredient A, 8.0% pea starch and 2.5% corn starch. Sample 2 is an example of a dairy analogue comprising the mycelium ingredient A, about 3.0% wheat starch and 2.5% corn starch. Spreadability was measured as well as a puncture test was performed. It was observed that the lower the starch content, the softest the texture becomes and thus a lower maximum force is needed for spreading the crème cheese dairy analogue i.e., the lowest force (N) needed to spread i.e., lowest positive area (N·s), and lowest force to puncture (N), and these values increase with increasing the starch content. But this usually depends on the starch type and the combination of starches used.

TABLE 8
Spreadability data for mycelium-based cream cheeses

| | | | | | | |
|---|---|---|---|---|---|---|
| | Max Force | Positive Area | Neg. Force | Neg. Area | Ratio | Ratio |
| Test ID | (N) | (N · s) | (N) | (N · s) | Force | Area |

spreadability 1 (sample 1)

| | | | | | | |
|---|---|---|---|---|---|---|
| spreadability 1_1 | 32.280 | 66.389 | −24.249 | −23.634 | −75.120 | −35.599 |
| Spreadability 1_2 | 27.737 | 53.913 | −21.360 | −27.791 | −77.009 | −51.547 |
| Spreadability 1_3 | 27.936 | 56.545 | −20.789 | −21.679 | −74.415 | −38.339 |
| Spreadability 1_4 | 21.255 | 46.830 | −15.383 | −21.816 | −72.375 | −46.586 |
| Spreadability 1_5 | 31.200 | 63.497 | −23.553 | −25.137 | −75.491 | −39.587 |
| Average | 28.082 | 57.435 | −21.067 | −24.011 | −74.882 | −42.332 |
| S.D. | 4.304 | 7.788 | 3.492 | 2.547 | 1.692 | 6.554 |
| Coef. of Variation | 15.326 | 13.559 | −16.575 | −10.608 | −2.260 | −15.483 |

Spreadability 2 (sample 2)

| | | | | | | |
|---|---|---|---|---|---|---|
| Spreadability 2_1 | 7.016 | 12.259 | −4.683 | −4.809 | −66.741 | −39.233 |
| Spreadability 2_2 | 7.235 | 12.935 | −5.252 | −5.350 | −72.601 | −41.357 |
| Spreadability 2-3 | 6.891 | 12.663 | −4.736 | −5.183 | −68.728 | −40.932 |
| Spreadability 2_4 | 6.619 | 11.763 | −4.804 | −5.286 | −72.569 | −44.937 |
| Spreadability 2_5 | 6.521 | 11.925 | −4.804 | −5.666 | −73.676 | −47.514 |
| Average | 6.856 | 12.309 | −4.856 | −5.259 | −70.863 | −42.795 |
| S.D. | 0.291 | 0.491 | 0.227 | 0.309 | 2.975 | 3.357 |
| Coef. of Variation | 4.242 | 3.992 | −4.684 | −5.879 | −4.198 | −7.845 |

TABLE 9
Puncture data for mycelium-based cream cheeses

| | | | | | |
|---|---|---|---|---|---|
| | Max + | | Positive | Neg. | Force at |
| | Force | Max- | Area | Area | the Target |
| Test ID | (N) | Force | (N · s) | (N · s) | (N) |

Puncture 1 (sample 1)

| | | | | | |
|---|---|---|---|---|---|
| Puncture 1_1 | 3.749 | −2.026 | 57.314 | −2.754 | 3.749 |
| Puncture 1_2 | 3.909 | −2.641 | 62.602 | −3.094 | 3.909 |
| Puncture 1_3 | 4.149 | −1.540 | 61.040 | −2.943 | 4.148 |
| Average | 3.936 | −2.069 | 60.319 | −2.931 | 3.936 |
| S.D. | 0.201 | 0.552 | 2.717 | 0.171 | 0.201 |
| Coef. of | 5.106 | −26.660 | 4.504 | −5.822 | 5.107 |
| Variation | | | | | |

Puncture 2 (sample 2)

| | | | | | |
|---|---|---|---|---|---|
| Puncture 2_1 | 0.660 | −0.345 | 10.720 | −0.632 | 0.659 |
| Puncture 2_2 | 0.604 | −0.327 | 9.481 | −0.549 | 0.601 |
| Puncture 2_3 | 0.595 | −0.347 | 9.388 | −0.590 | 0.595 |
| Average | 0.620 | −0.340 | 9.863 | −0.591 | 0.618 |
| S.D. | 0.036 | 0.011 | 0.744 | 0.041 | 0.036 |
| Coef. of | 5.754 | −3.132 | 7.543 | −7.000 | 5.778 |
| Variation | | | | | |

### Cutting Strength Methodology

Cutting strength were determined using a TA.XTplus100 Texture Analyzer (Stable Micro System, Surrey, United Kingdom). A 50 kg load cell was used for the tests. Samples were analyzed at cooling temperature (≈7° C.). The meatballs were cut 80% of their original height by blade. The results were given as an average of the maximum forces (N) required to cut through the samples. In addition to the force, the areas under the curve were calculated (N·s) as well. Force-time curves were recorded with the maximum force being taken as maximum cutting strength. The area under the positive curve (N·s) represented the total amount of force.

TABLE 10
Cutting strength for a soft mycelium-based meat analogue

| | | | |
|---|---|---|---|
| | Gradient | Max | Area |
| Test ID | (N/mm) | Force (N) | (N · s) |

Soft Meat Analogue Cutting Strength

| | | | |
|---|---|---|---|
| Sample 1 | 0.655 | 6.559 | 50.407 |
| Sample 2 | 0.492 | 6.331 | 50.689 |
| Sample 3 | 0.524 | 6.058 | 47.952 |
| Sample 4 | 0.514 | 5.701 | 47.607 |
| Sample 5 | 0.743 | 7.018 | 53.353 |
| Average | 0.586 | 6.334 | 50.002 |
| S.D. | 0.109 | 0.499 | 2.335 |
| Coef. of | 18.555 | 7.876 | 4.669 |
| Variation | | | |

### Texture Profile Analysis (TPA) Methodology

TPA was performed using a TA.XTplus100 Texture Analyzer (Stable Micro System, Surrey, United Kingdom). A 50 kg load cell was used for the tests. Samples were analyzed at cooling temperature (≈7° C.). The unfried meatballs, cooked at about 85C for 5 to 10 mins, were compressed by 60% of their original height twice in a row, with a test speed of 1 mm/sec. Therefore, the compression plate (SMS P/75) was used. The results were given as an average of the maximum forces (N) required to cut through the samples.

Force-time curves were recorded, with the maximum force (N) at the first peak being taken as sample hardness. The area under the first negative peak (N·s) represented the adhesiveness of the sample. The height recovery represents the sample springiness (%) and the 2^{nd} area divided by the 1^{st} area (%) demonstrates the cohesiveness of the sample. The values of the gumminess (N) result from the product of hardness (N) and cohesiveness (%) while the chewiness (N) is the result of multiplying the gumminess (N) and springiness (%).

TABLE 11
TPA data for a soft mycelium-based meat analogue

| | | | | | | | |
|---|---|---|---|---|---|---|---|
| | Sample | | | | | | |
| | height | Hardness | Springiness | Cohesiveness | Gumminess | Chewiness | |
| Test ID* | (mm) | (N) | (%) | (%) | (N) | (N) | Resilience |

Soft Meat Analogue TPA Data

| | | | | | | | |
|---|---|---|---|---|---|---|---|
| Sample 1 | 28.414 | 40.927 | 71.336 | 35.691 | 14.608 | 10.431 | 0.123 |
| Sample 2 | 27.506 | 44.426 | 74.409 | 35.570 | 15.802 | 11.758 | 0.121 |
| Sample 3 | 29.899 | 33.755 | 70.696 | 35.857 | 12.104 | 8.557 | 0.129 |
| Average | 28.606 | 39.703 | 72.147 | 35.706 | 14.171 | 10.245 | 0.124 |
| S.D. | 1.208 | 5.440 | 1.985 | 0.144 | 1.888 | 1.608 | 0.004 |
| Coef. of | 4.223 | 13.702 | 2.751 | 0.404 | 23.877 | 15.695 | 3.127 |
| Variation | | | | | | | |

### Recipe Preparation for Mycelium-Based Cream Cheese Via Three Different Methods:

### Acidification Through Ingredients

Homogenize 10-50 wt. % of Pleurotus Pulmonarius mycelium (edible fibrous mycelium) with about 55 wt. % potable Water, about 0 to 7 wt. % of cashew nuts (plant derived fat component), 10 to 40 wt. % of Cocos nucifera oil (plant derived fat component), 2 wt. % at most of table salt (sodium chloride source), 2 wt. % at most of yeast flakes, 5 wt. % at most of sucrose (plant derived saccharide source), 5 wt. % at most of lemon juice (natural plant derived acidity source), 5 wt. % at most of citric acid.

Under constant blending, heat the homogenized slurry for a maximum of 60 seconds at a temperature of 75 to 95° C. To coagulate the homogenized slurry, a beforehand prepared coagulation agent solution is administered during this precedingly circumscribed heating step as soon as the temperature has reached 25° C. to 40° C. This coagulation agent solution comprises of 15 wt. % at most of each of the following: water, a wheat starch as well as a hydrolyzed corn starch.

Swiftly remove the obtained slurry from the heat source and subsequently allow the mixture to settle to room temperature (21° C.). Once room temperature is reached, store the coagulated slurry in a dark, and cool environment, preferably between 4° C. to 7° C.

### Acidification Through Microbial Fermentation

Homogenize 10-50 wt. % of Pleurotus Pulmonarius mycelium (edible fibrous mycelium) with about 55 wt. % potable Water, about 0 to 7 wt. % of cashew nuts (plant derived fat component), 10 to 40 wt. % of Cocos nucifera oil (plant derived fat component), 2 wt. % at most of table salt (sodium chloride source), 2 wt. % at most of yeast flakes, 5 wt. % at most of sucrose (plant derived saccharide source).

Under constant blending, heat the homogenized slurry for a maximum of 60 seconds at a temperature of 75 to 95° C. To coagulate the homogenized slurry, a beforehand prepared coagulation agent solution is administered during this precedingly circumscribed heating step as soon as the temperature has reached 25° C. to 40° C. This coagulation agent solution comprises of 15 wt. % at most of each of the following: water, a wheat starch as well as a hydrolyzed corn starch.

Swiftly remove the obtained slurry from the heat source and subsequently allow the mixture to settle to room temperature (21° C.). Once the obtained mixture reaches a temperature of 40° C. or less, add 0.1 g to 0.25 g of acid-forming bacteria, in particular lactic acid bacteria to induce microbial acidification. To provide a hospital environment for the microorganisms, the sample is placed for 150 minutes into a controlled temperature environment of 28° C. (e.g., water bath, or incubator). Depending on intensity of fermentation, sample can be placed from 1 to 6 hours at a temperature of 20 to 45° C. After the microbial fermentation is completed, store the coagulated slurry in a dark, and cool environment, preferably between 4° C. to 7° C.

### Creme Cheese Based on Two Fungal Stains

Follow the same instructions seen in Example 1 and Example 2, solely replacing a fraction of the Pleurotus Pulmonarius edible fibrous mycelium with Morchella rufobrunnea edible fibrous mycelium. Assuming the above listed quantities, 60-90 wt. % of total mycelium would be from Pleurotus Pulmonarius mycelium, and 10-40 wt. % of the total mycelium ingredients would be from Morchella rufobrunnea mycelium (edible fibrous mycelium).

### Recipe Preparation for a Vegetarian Mycelium-Based Meat Analogue Composition:

Blend 50 to 95 wt. % of Pleurotus Pulmonarius mycelium (edible fibrous mycelium) and add it to 1 to 40 wt. % at most from each of the following ingredients: canola oil, salt, egg white and wheat gluten. Optionally add 1 to 40 wt. % at most from each of the following ingredients: methylcellulose, hydrocolloids, texturized vegetable proteins, starch-based ingredients, and flavor components. The obtained dough is then shaped via a forming machine and/or an extruder or a combination thereof, to have the final product as meat balls or sausages or extruded product.

### Recipe Preparation for a Vegan Mycelium-Based Meat Analogue Composition:

Blend 50 to 95 wt. % of Pleurotus Pulmonarius mycelium (edible fibrous mycelium) and add it to 1 to 40 wt. % at most from each of the following ingredients: canola oil, salt, and wheat gluten. The missing egg white is accounted for by the addition of additional dry mycelium, with an equivalence ratio of 1 to 1.4, respectively. Optionally add 1 to 40 wt. % at most from each of the following ingredients: methylcellulose, hydrocolloids, texturized vegetable proteins, starch-based ingredients, and flavor components The obtained dough is then shaped via a forming machine and/or an extruder or a combination thereof, to have the final product as meat balls or sausages or extruded product.

Further examples and embodiments of the present invention are disclosed in the following numbered items:
1. An edible mycelium ingredient A derived from submerged fermentation having an elemental composition of a C:N ratio of mycelium ranging between 6 and 8.
2. An edible mycelium ingredient B derived from submerged fermentation having an elemental composition of a C:N ratio of mycelium ranging between 8 and 12.
3. An edible mycelium ingredient C derived from submerged fermentation having an elemental composition of a C:N ratio of mycelium ranging between 2 and 6.
4. The edible mycelium ingredients of item 1, wherein the inherent RNA level is below 1.88 wt. % on a dry basis, without a further process step to reduce RNA.
5. The edible mycelium ingredients of item 2, wherein the inherent RNA level is below 1.65 wt. % on a dry basis, without a further process step to reduce RNA.
6. The edible mycelium ingredients of items 1-3, where in the inherent RNA level is 2 wt. % at most on a dry basis, without a further process step to reduce RNA.
7. The edible mycelium ingredient of item 1, wherein the mycelia have an ergothioneine content ranges between 70 and 100 mg/kg.
8. The edible mycelium ingredient of item 2, wherein the mycelia have an ergothioneine content ranges between 110 and 150 mg/kg.
9. The edible mycelium ingredient of item 3, wherein the mycelia have an ergothioneine content ranges between 380 and 455 mg/kg.
10. The edible mycelium ingredients of item 1-3, wherein 5′-inosine monophosphate (IMP) is 0 g/kg.
11. The edible mycelium ingredient of item 1, wherein the content of umami 5′ nucleotides (5′NMP) containing 5′-guanosine monophosphate (GMP) and 5′-adenosine monophosphate (AMP) is up to 6 g/kg.
12. The edible mycelium ingredient of item 2, wherein the content umami 5′ nucleotides (5′NMP) containing 5′-guanosine monophosphate (GMP) and 5′-adenosine monophosphate (AMP) is up to 3 g/kg.
13. The edible mycelium ingredient of item 3, wherein the content of umami 5′ nucleotides (5′NMP) containing 5′-guanosine monophosphate (GMP) and 5′-adenosine monophosphate (AMP) is up to 20 g/kg.
14. The edible mycelium ingredients of any one of items 1-3, wherein the umami taste can be further intensified by at least 60% upon an enzymatic treatment of AMP to IMP.
15. The edible mycelium ingredient of item 3, wherein a concentration of uridine monophosphate (UMP) is present of 3.56 g/kg.
16. The edible mycelium ingredients of any one of items 1-3, wherein the amount of the branched-chain amino acids (BCAAs) is at least about 19 wt. % of the total amount of amino acids present.
17. The edible mycelium ingredients of any one of items 1-3, wherein the amount of the umami amino acids is at least about 19 wt. % of the total amount of amino acids present.
18. The edible mycelium ingredients of items 1-3, wherein the amount of the essential amino acids is at least about 40 wt. % of the total amount of amino acids present.
19. The edible mycelium ingredient of item 1, wherein the amount of the BCAAs and the amount of umami amino acids range between 40 and 100 mg/g each respectively.
20. The edible mycelium ingredient of item 2, wherein the amount of the BCAAs and the amount of umami amino acids range between 20 and 80 mg/g each respectively.
21. The edible mycelium ingredient of item 3, wherein the amount of the BCAAs ranges 50 and 100 mg/g and the amount of umami amino acids ranges between 70 and 100 mg/g.
22. The edible mycelium ingredient of item 1, having an EUC concentration of at least 500%.
23. The edible mycelium ingredient of item 2, having an EUC concentration of at least 400%, preferably about 460%.
24. The edible mycelium ingredient of item 3, having an EUC concentration of at least 500%, more preferably at least 5000%, most preferably at least 10000%.
25. The edible mycelium ingredients of item 1-2, having a thermal stability up to 220° C. at most under N₂ atmosphere.
26. The edible mycelium ingredient of item 3, having a thermal stability up to 190° C. at most ° C. under N₂ atmosphere.
27. The edible mycelium ingredients of any one of items 1-3, wherein the calorific value ranges between 300 and 600 Kcal/100 g.
28. The edible mycelium ingredient of item 1, having an insoluble fiber content between 30 and 60 wt. %, preferably between 30 and 40 wt. %.
29. The edible mycelium ingredient of item 2, having an insoluble fiber content between 40 and 60 wt. %, preferably between 40 and 50 wt. %.
30. The edible mycelium ingredient of item 3, having an insoluble fiber content between 10 and 40 wt. %, preferably between 20 and 30 wt. %.
31. The edible mycelium ingredient of item 1, having a protein content between 30 and 50 wt. %, preferably between 30 and 40 wt. %.
32. The edible mycelium ingredient of item 2, having a protein content between 30 and 50 wt. %, preferably between 30 and 40 wt. %.
33. The edible mycelium ingredient of item 3, having a protein content between 30 and 65 wt. %, preferably between 45 and 65 wt. %.
34. The edible mycelium ingredients of item 1-2, having a content of ergosterol between 2 and 4 mg/g.
35. The edible mycelium ingredient of item 3, having a content of ergosterol between 4 and 7 mg/g.
36. The edible mycelium ingredients of any one of items 1-3, having a carbohydrate content of 1% at most.
37. The edible mycelium ingredients of any one of items 1-3, having a content of uronic acids between 0.1 and 5 wt. %.
38. The edible mycelium ingredients of any one of items 1-3, wherein the chitin content ranges between 6 and 11 wt. %.
39. The edible mycelium ingredients of any one of items 1-3, wherein the beta-glucan content is at least 80% of the total glucans.
40. The edible mycelium ingredient of item 1, wherein the total glucans content ranges between 20 and 35 wt. %.
41. The edible mycelium ingredient of item 2, wherein the total glucans content ranges between 25 and 50 wt. %.
42. The edible mycelium ingredient of item 3, wherein the total glucans content ranges between 10 and 20 wt. %.
43. The edible mycelium ingredients of any one of items 1-3, wherein at least 96 wt. % of their polyunsaturated fatty acids comprises linoleic acid.
44. The edible mycelium ingredient of item 3, having an enriched omega-6 fatty acid (linoleic acid) ranging between 2 and 5 wt. %.
45. The edible mycelium ingredients of item 1-2, having a fat content of at most 3 wt. %.
46. The edible mycelium ingredient of item 3, having an enriched fat content wherein the fat content is at most at most 8 wt. %.
47. The edible mycelium ingredients of any one of items 1 to 3, having a total phenolic content (TPC) ranging between 1 and 15 GAE/g and a total flavonoid content (TFC) ranging between 1 and 15 mg QE/g, wherein the flavonoid content of the mycelium ingredient A constitutes around 35% to 60% of the total phenolic content, the flavonoid content of the mycelium ingredient B constitutes around 70 to 95% of the total phenolic content, the flavonoid content of the mycelium ingredient C constitute around 50 to 80% of the total phenolic content.
48. The edible mycelium ingredient of item 1, when freeze-dried, has a specific pore volume of 9 cm3/g and a median pore diameter of 44.5 μm and a BET surface area of 0.79 m2/g.
49. The edible mycelium ingredient of item 2, when freeze-dried, has a specific pore volume of 4.94 cm3/g and a median pore diameter of 143 μm and a BET surface area of 0.67 m2/g.
50. The edible mycelium ingredient of item 2, when freeze-dried, has a specific pore volume of 2.46 cm3/g and a median pore diameter of 7.1 μm and a BET surface area of 1.59 m2/g.
51. The edible mycelium ingredient of item 1, when freeze dried and for pore diameters below 1 mm, 58% of its pore-volume corresponds to a pore diameter between 1000 and 30 μm with the most frequent pore diameter in the range of 85-185 μm and 42% of the pore-volume corresponds to a pore diameter between 30-2 μm having the most frequent pore diameter peak is equal to 16 μm, which is the most frequent pore diameter in the range of 1000-2 μm.
52. The edible mycelium ingredient of item 2, when freeze dried and for pore diameters below 1 mm, 81.5% of its pore-volume corresponds to a pore diameter between 1000 and 30 μm with the most frequent pore diameter in the range of 147 μm, which is also the most frequent pore diameter in the range of 1000-2 μm and 18.5% of the pore-volume corresponds to a pore diameter between 30-2 μm having the most frequent pore diameter peak is equal to 15 μm.
53. The edible mycelium ingredient of item 3, when freeze dried and for pore diameters below 1 mm, 20% of its pore-volume corresponds to a pore diameter between 1000 and 20 μm and 80% of the pore-volume corresponds to a pore diameter between 20-2 μm having the most frequent pore diameter peak is equal to 5.5 μm.
54. The edible mycelium ingredients of any one of items 1-3, where in the mycelium has a shear force of at least 15 N and water holding capacity ranging between 20-90% and a water release of 25 to 70%.
55. A method for producing the mycelium ingredients of any one of items 1-3 via submerged fermentation, wherein the fermentation medium comprises of 5 to 60 g/L of carbon source, 0.1 to 60 g/L nitrogen source, 0.01-15 g/L of minerals, and 0.01-50 mg/L of vitamins.
56. The method of item 55, wherein the production of ingredient A of item 1 comprises the step of culturing at least one fungal species in a defined medium comprising of multiple amino acids and vitamin sources based on a complex nitrogen source and at least one carbon source, minerals, wherein the carbon to nitrogen ratio in this medium ranges between 14 and 19.
57. The method of item 55, wherein the production of ingredient B of item 2 comprises the step of culturing at least one fungal species in a minimal synthetic medium comprising one amino acid and one vitamin, wherein the carbon to nitrogen ratio in this medium ranges between 16 and 23.
58. The method of item 55, wherein the production of ingredient C of item 3 comprises the step of culturing at least one fungal species in a natural synthetic medium comprising spent grain C5-sugar extract wherein the carbon to nitrogen ratio in this medium ranges between 10 and 25 and wherein at least 35 wt. % of extracted spent grains ranges between 2 and 4 mm.
59. The method of any one of items 55-58, wherein the at least one fungal species is selected from Basidiomycota, Ascomycota, Pezizomycotina, Agaromycotina, Pezizomycetes, Agaricomycetes, Sordariomycetes, Pezizales, Boletales, Cantharellales, Agaricales, Polyporales, Russulales, Auriculariales, Hypocreales, Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, and Fistulinacea.
60. The method of any one of items 55-58, wherein at least one fungal species is combined with another edible fungi, algae, bacteria, plant cells, archaea cells, animal cells, fat cells or a combination thereof.
61. The methods of any one of items 55-58 further comprising the step of recovering the supernatant from the culture medium.
62. The method of item 61, wherein the recovering comprises the step of crystallizing or precipitating the obtained supernatant.
63. A method for producing a soft or hard meat analogue composition comprising of at least one of the mycelium ingredients of any one of items 1-3, comprising the method of any one of items 55 to 58 for producing the respective mycelium ingredient and further comprising the step of preparing such meat analogue composition by mixing at least one of the mycelium ingredient of any one of items 1-3 from at least one fungal strain with a composition comprising of at least one protein rich ingredient, at least one plant-based lipid rich ingredient, and optionally at least one compositional ingredient.
64. A method for producing a dairy analogue composition comprising of at least one of the mycelium ingredients of any one of items 1-3 from at least one fungal strain, comprising the method of any one of items 55 to 58 for producing the respective mycelium ingredient and further comprising the step of preparing such a dairy analogue composition by (1) forming a slurry comprising at least one of the mycelium ingredient of any one of items 1-3 with a composition comprising of at least one protein rich ingredient, at least one plant-based lipid rich ingredient, and at least one compositional ingredient, and (2) mixing the slurry with at least one compositional ingredient, specifically a texturizing agent or thickener or a carbohydrate-rich ingredient.
65. The edible products of item 63 or 64, further comprising an edible plant, or algae-based fat component, a fat component derived from fungi or yeast, in the range of up to 25 wt. %, preferably in the range from 1 to 5 wt. %
66. The edible products of item 64 or 64, wherein the edible plant-based lipid rich ingredient component is selected from nuts, coconut oil, sunflower oil, rapeseed oil, palm oil, cotton seed oil, olive oil, canola oil, algae oil, and/or oleaginous yeast-derived oils.
67. The edible products of item 64, wherein the at least one protein rich ingredient or the at least one compositional ingredient are selected from agar-agar, egg white, yeast flakes, edible starch, guar gum, locust bean gum, wheat gluten, saccharide source, cellulose or derivatives of it, lemon juice, colorants and/or a flavorant, such as salt, extracts and/or spices.
68. The obtained meat analogue of item 63, comprising a mycelium mass in the range of 1 wt. % to 99 wt. % and water in the range of up to 99 wt. %, wherein the edible edible fibrous mycelium has an insoluble fiber content of 40 to 50 wt. %, preferably about 45% w/W.
69. The obtained dairy analogue of item 64, comprising a mycelium mass in the range of 1 wt. % to 99 wt. % and water in the range of up to 99 wt. %, where in the edible edible fibrous mycelium has an insoluble fiber content of at least about 40 wt. %.
70. The edible meat analogue of item 64, comprising the fibrous mycelium mass in the range of 50 wt. % to 95 wt. %.
71. The edible dairy analogue of item 63, comprising the fibrous mycelium mass in the range of 1 wt. % to 50 wt. %.
72. The products of item 63 or 64, characterized in that such products could have a soft or non-soft/harder texture.
73. The food product of item 63, characterized in a soft texture, preferably has a hardness of 10-55N, springiness of 35-85%, cohesiveness of 15-70%, gumminess of 1-40N, a chewiness of 0.3-35N, a cutting strength of 1-25N and an adhesiveness of 0 N·s to-0.3 N·s.
74. The food product of item 63, characterized in a non-soft or harder texture, preferably has a hardness of 30-100N, springiness of 20-70%, cohesiveness of 20-85%, gumminess of 6-85N, a chewiness of 1-60N, a cutting strength of 1-50N.
75. The food product of item 64, characterized in a soft texture, preferably has a firmness of 1 to 20N, a spreadability of 30 to 100 N·s, a stickiness of-15 to-100N and a puncture force of 1 to 30 N·s.
76. The food product of item 64, characterized in a non-soft or harder texture, preferably has a firmness of 20 to 100N, a spreadability of 1 to 20 N·s, a stickiness of-1 to-14N and a puncture force of 40 to 100 N·s.

Further embodiments of the invention are disclosed in the following numbered clauses.
1. An edible mycelium ingredient C derived from submerged fermentation having an elemental composition of a C:N ratio of mycelium ranging between 2 and 6.
2. The edible mycelium ingredients of clause 1,
   wherein the inherent RNA level is 4 wt. % at most, preferably 2 wt. % at most on a dry basis, without a further process step to reduce RNA, and/or wherein the content of umami 5′ nucleotides (5′NMP) containing 5′-guanosine monophosphate (GMP) and 5′-adenosine monophosphate (AMP) is up to 40 g/k at most, preferably 20 g/kg at most, and/or
   wherein 5′-inosine monophosphate (IMP) is 0 g/kg, and/or
   wherein the umami taste coming from 5′-nucleotides can be further intensified by at least 40% upon an enzymatic treatment, preferably with 5′-Adenylic deaminase, to convert AMP to IMP, and/or
   wherein the amount of the branched-chain amino acids (BCAAs) is at least about 19 wt. % of the total amount of amino acids present, and/or
   wherein the amount of the essential amino acids is at least about 40 wt. % of the total amount of amino acids present, and/or
   wherein the amount of the BCAAs ranges 50 and 150 mg/g and the amount of umami amino acids ranges between 70 and 100 mg/g.
3. The edible mycelium ingredient of clause 1 or 2,
   wherein the mycelia have an ergothioneine content ranging between 350 and 800 mg/kg.
4. The edible mycelium ingredient of clause 1 or 3, wherein the amount of the umami amino acids is at least about 19 wt. % of the total amount of amino acids present.
5. The edible mycelium ingredient of any one of clauses 1 to 4, having an EUC concentration of at least 500%, more preferably at least 5000%, most preferably at least 10000%.
6. The edible mycelium ingredient of any one of clauses 1 to 5,
   wherein at least 96 wt. % of their polyunsaturated fatty acids comprises linoleic acid, preferably having an enriched omega-6 fatty acid (linoleic acid) ranging between 2 and 5 wt. %, and/or
   having an enriched fat content wherein the fat content is at most 8 wt. %, and/or
   having a DPPH radical-scavenging activity correlating with the TPC ranging between 1 and 15 mg/ml, and/or wherein the TPC, TFC and polyphenols contents range between 1 and 15 mg GAE/g, 1 and 15 mg QE/g and 100 and 1000 mg/kg, respectively, wherein the flavonoid content constitutes around 70 to 95% of the total phenolic content and wherein catechin and protocatechuic acid constitute each around 45 to 55% of the total polyphenols.
7. The edible mycelium ingredients of any one of clauses 1 to 6,
   wherein the calorific value ranges between 300 and 600 Kcal/100 g with the ingredients having a thermal stability under N₂ atmosphere up to 190° C. at most, and/or
   wherein the insoluble fiber content is between 10 and 40 wt. %, preferably between 20 and 30 wt. %, and/or wherein the protein content between 30 and 65 wt. %, preferably between 45 and 65 wt. %, and/or
   wherein the content of ergosterol is between 4 and 7 mg/g, and/or wherein the carbohydrate content is 5 wt. % at most, and/or
   wherein the content of uronic acids is between 0.1 and 5 wt. %,
   and/or wherein the chitin content ranges between 6 and 11 wt. %, and/or wherein the beta-glucan content is at least 80% of the total glucans, preferably wherein the total glucans content ranges between 10 and 20 wt. %.
8. The edible mycelium ingredient of any one of clauses 1 to 7,
   wherein, when freeze dried, for pore diameters below 1 mm, about 15 to 25%, preferably about 20% of its pore-volume corresponds to a pore diameter between 1000 and 20 μm and about 75 to 85%, preferably about 80% of the pore-volume corresponds to a pore diameter between 20-2 μm having the most frequent pore diameter peak is equal to 5.5 μm, and/or wherein the mycelium has a shear force of at least 15 N and water holding capacity ranging between 20-90% and a water release of 25 to 70%.
9. A method for producing the mycelium ingredient of any one of clauses 1 to 8 via submerged fermentation, wherein the fermentation medium comprises of 5 to 60 g/L of carbon source, 0.1 to 60 g/L nitrogen source, 0.01-15 g/L of minerals, and 0.01-50 mg/L of vitamins wherein the production of ingredient C comprises the step of culturing at least one fungal species in a natural synthetic medium comprising spent grain C5-sugar extract wherein the carbon to nitrogen ratio in this medium ranges between 10 and 25 and wherein at least 35 wt. % of extracted spent grains ranges between 2 and 4 mm, preferably wherein the particle size distribution is determined by sieving performed by an air-jet sieving method, following DIN 10765 2016-07, after using an automatic sieving tower, i.e., a vibrating sieving method to get rid of particle sizes above 4 mm.
10. The method of clause 9, wherein the at least one fungal species is selected from Basidiomycota, Ascomycota, Pezizomycotina, Agaromycotina, Pezizomycetes, Agaricomycetes, Sordariomycetes, Pezizales, Boletales, Cantharellales, Agaricales, Polyporales, Russulales, Auriculariales, Hypocreales, Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, and Fistulinacea, preferably wherein the at least one fungal strain is selected from Basidiomycota, Ascomycota, Pezizomycotina, Agaromycotina, Agaricomycetes, Pezizales, Boletales, Cantharellales, Agaricales, Polyporales, Russulales, Auriculariales, Hypocreales, Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, and Fistulinacea, preferably wherein the mycelium mass is obtained from Pleurotus pulmonarius, Pleurotus ostreatus, Pleurotus florida, Pleurotus citrinopileatus, Pleurotus salmoneostramineus, Morchella esculenta, Morchella angusticeps, Morchella deliciosa, and/or Morchella rufobrunnea, preferably wherein the mycelium mass is obtained from Pleurotus pulmonarius and/or Morchella rufobrunnea, optionally wherein the at least one fungal species is combined with another edible fungi, algae, bacteria, plant cells, archaea cells, animal cells, fat cells or a combination thereof.
11. The method of clause 9 or 10, further comprising the step of recovering the supernatant from the culture medium or part of it, optionally wherein the recovering comprises the step of crystallizing or precipitating the obtained supernatant.
12. A mycelial ingredient obtainable according to the method of any one of clauses 9 to 11.
13. A method for producing a soft or hard meat analogue composition comprising the mycelium ingredient of any one of clauses 1 to 8 or 12, comprising the step of preparing such meat analogue composition by adding said mycelium ingredient to a composition comprising of at least one protein rich ingredient, at least one plant-based lipid rich ingredient, and optionally at least one compositional ingredient.
14. A method for producing a dairy analogue composition comprising the mycelium ingredient of any one of clauses 1 to 8 or 12 comprising the step of preparing such a dairy analogue composition by (1) forming a slurry comprising said mycelium ingredient with a composition comprising of at least one protein rich ingredient, at least one plant-based lipid rich ingredient, and at least one compositional ingredient, and (2) mixing the slurry with at least one compositional ingredient, specifically a texturizing agent or thickener or a carbohydrate-rich ingredient.
15. An edible product obtainable in the method of clause 13 or 14,
   preferably wherein the food product of clause 13 characterized in a soft texture, preferably has a hardness of 10-55N, springiness of 35-85%, cohesiveness of 15-70%, gumminess of 1-40N, a chewiness of 0.3-35N, a cutting strength of 1-25N and an adhesiveness of 0 N·s to −0.3 N·s, and/or
   preferably wherein the food product of clause 13 characterized in a non-soft or harder texture,
   preferably has a hardness of 30-100N, springiness of 20-70%, cohesiveness of 20-85%, gumminess of 6-85N, a chewiness of 1-60N, a cutting strength of 1-50N, and/or
   preferably wherein the food product of clause 14 characterized in a soft texture, preferably has a firmness of 1 to 20N, a spreadability of 30 to 100 N·s, a stickiness of-15 to-100N and a puncture force of 1 to 30 N·s, and/or
   preferably wherein the food product of clause 14 characterized in a non-soft or harder texture,
   preferably has a firmness of 20 to 100N, a spreadability of 1 to 20 N·s, a stickiness of-1 to-14N and a puncture force of 40 to 100 N·s.

Further embodiments of the invention are disclosed in the following numbered paragraphs.
1. An edible mycelium ingredient derived from submerged fermentation having an elemental composition of a C:N ratio of mycelium ranging between 6 and 12.
2. The edible mycelium ingredient of paragraph 1, being an edible mycelium ingredient A derived from submerged fermentation in a defined medium having an elemental composition of a C:N ratio of mycelium ranging between 6 and 8.
3. The edible mycelium ingredient of paragraph 1, being an edible mycelium ingredient B derived from submerged fermentation in a synthetic medium having an elemental composition of a C:N ratio of mycelium ranging between 8 and 12.
4. The edible mycelium ingredient of paragraph 2,
   wherein the inherent RNA level is 2 wt. % at most on a dry basis, without a further process step to reduce RNA, and/or
   wherein the mycelia have an ergothioneine content ranging between 70 and 270 mg/kg achieved in 5 days, and/or
   wherein the content of umami 5′ nucleotides (5′NMP) containing 5′-guanosine monophosphate (GMP) and 5′-adenosine monophosphate (AMP) 20 g/kg at most, and/or
   wherein the amount of the BCAAs and the amount of umami amino acids range between 40 and 100 mg/g each respectively, and/or
   wherein the calorific value ranges between 300 and 600 Kcal/100 g, and/or
   having an insoluble fiber content between 30 and 60 wt. %, preferably between 30 and 40 wt. %, and/or
   having a protein content between 30 and 50 wt. %, preferably between 30 and 40 wt. %, and/or
   wherein the total glucans content ranges between 20 and 35 wt. %, and/or
   wherein, when freeze dried and for pore diameters below 1 mm, 55 to 65%, preferably about 58% of its pore-volume corresponds to a pore diameter between 1000 and 30 μm with the most frequent pore diameter in the range of 85-185 μm and 35 to 45%, preferably about 42% of the pore-volume corresponds to a pore diameter between 30-2 μm having the most frequent pore diameter peak is equal to 16 μm, which is the most frequent pore diameter in the range of 1000-2 μm, and/or
   wherein the flavonoid content constitutes about 35% to 60% of the total phenolic content, and/or,
   wherein catechin and protocatechuic acid constitute each around 35 to 45% of the total polyphenols.
5. The edible mycelium ingredient of paragraph 2 or 4, having an EUC concentration of at least 500%.
6. The edible mycelium ingredient of paragraph 3,
   wherein the inherent RNA level is 2 wt. % at most on a dry basis, without a further process step to reduce RNA, and/or
   wherein the mycelia have an ergothioneine content ranging between 100 and 350 mg/kg, and/or
   wherein the content umami 5′ nucleotides (5′NMP) containing 5′-guanosine monophosphate (GMP) and 5′-adenosine monophosphate (AMP) is 15 g/kg at most, and/or
   wherein the amount of the BCAAs and the amount of umami amino acids range between 20 and 80 mg/g each respectively, and/or
   having an insoluble fiber content between 40 and 60 wt. %, preferably between 40 and 50 wt. %, and/or
   having a protein content between 30 and 50 wt. %, preferably between 30 and 40 wt. %, and/or
   wherein the total glucans content ranges between 25 and 50 wt. %, and/or
   wherein, when freeze dried and for pore diameters below 1 mm, 75 to 85%, preferably about 81.5% of its pore-volume corresponds to a pore diameter between 1000 and 30 μm with the most frequent pore diameter in the range of 147 μm, which is also the most frequent pore diameter in the range of 1000-2 μm and 15 to 25%, preferably about 18.5% of the pore-volume corresponds to a pore diameter between 30-2 μm having the most frequent pore diameter peak is equal to 15 μm, and/or
   wherein the flavonoid content constitutes about 70 to 95% of the total phenolic content and/or,
   wherein catechin and protocatechuic acid constitute about 25 to 35% and about 35 to 45% of the total polyphenols, respectively.
7. The edible mycelium ingredient of any one of paragraphs 1 to 6, wherein the DPPH radical-scavenging activity, the total phenolic content TPC, the total flavonoid content TFC and polyphenols contents range between 1 and 15 mg/ml correlating with the TPC, between 1 and 15 mg GAE/g, 1 and 15 mg QE/g, and 50 and 900 mg/kg, respectively.
8. The edible mycelium ingredient of any one of paragraphs 1 to 7, having a thermal stability up to 210 to 220° C. under N₂ atmosphere, and/or
   wherein 5′-inosine monophosphate (IMP) is 0 g/kg, and/or
   wherein the amount of the branched-chain amino acids (BCAAs) is at least about 19 wt. % of the total amount of amino acids present, and/or
   wherein the amount of the umami amino acids is at least about 19 wt. % of the total amount of amino acids present, and/or
   wherein the amount of the essential amino acids is at least about 40 wt. % of the total amount of amino acids present, and/or
   having a content of ergosterol between 2 and 4 mg/g, and/or
   having a carbohydrate content of 5% at most, and/or
   having a content of uronic acids between 0.1 and 5 wt. %, and/or
   wherein the chitin content ranges between 6 and 11 wt. %, and/or
   wherein the beta-glucan content is at least 80% of the total glucans, and/or
   having a fat content of at most 3 wt. %, and/or
   wherein the mycelium has a shear force of at least 15 N and water holding capacity ranging between 20-90% and a water release of 25 to 70%.
9. A method for producing the mycelium ingredients of any one of paragraphs 1 to 8 via submerged fermentation, wherein the fermentation medium comprises 5 to 60 g/L of carbon source, 0.1 to 60 g/L nitrogen source, 0.01-15 g/L of minerals, and 0.01-50 mg/L of vitamins wherein the carbon to nitrogen ratio in this medium ranges between 14 and 23, preferably 14 to 19 for medium A and 16 to 23 for medium B, characterized in that the at least one vitamin or the at least one amino acid used in synthetic medium B has an amount characterized by the ratio of said at least one vitamin or at least one amino acid used to the total amount of all vitamins or all amino acids present in the defined medium A in the range of 0.01 to 10, preferably between 0.1 and 10.
10. The method of paragraph 9, wherein the at least one fungal species is selected from Basidiomycota, Ascomycota, Pezizomycotina, Agaromycotina, Pezizomycetes, Agaricomycetes, Sordariomycetes, Pezizales, Boletales, Cantharellales, Agaricales, Polyporales, Russulales, Auriculariales, Hypocreales, Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, and Fistulinacea, preferably wherein the at least one fungal strain is selected from Basidiomycota, Ascomycota, Pezizomycotina, Agaromycotina, Agaricomycetes, Pezizales, Boletales, Cantharellales, Agaricales, Polyporales, Russulales, Auriculariales, Hypocreales, Morchellaceae, Tuberaceae, Pleurotaceae, Agaricaceae, Marasmiaceae, Cantharellaceae, Hydnaceae, Boletaceae, Meripilaceae, Polyporaceae, Strophariaceae, Lyophyllaceae, Tricholomataceae, Omphalotaceae, Physalacriaceae, Schizophyllaceae, Sclerodermataceae, Ganodermataceae, Sparassidaceae, Hericiaceae, Bondarzewiaceae, Cordycipitaceae, Auriculariaceae, and Fistulinacea, preferably wherein the mycelium mass is obtained from Pleurotus pulmonarius, Pleurotus ostreatus, Pleurotus florida, Pleurotus citrinopileatus, Pleurotus salmoneostramineus, Morchella esculenta, Morchella angusticeps, Morchella deliciosa, and/or Morchella rufobrunnea., preferably wherein the mycelium mass is obtained from Pleurotus pulmonarius and/or Morchella rufobrunnea,
   optionally wherein the at least one fungal species is combined with another edible fungi, algae, bacteria, plant cells, archaea cells, animal cells, fat cells or a combination thereof.
11. The method of paragraph 9 or 10, further comprising the step of recovering the supernatant from the culture medium or part of it, optionally wherein the recovering comprises the step of crystallizing or precipitating the obtained supernatant.
12. A mycelial ingredient obtainable according to the method of any one of paragraphs 9 to 11.
13. A method for producing a soft or hard meat analogue composition comprising the mycelium ingredient of any one of paragraphs 1 to 8 or 12, comprising the step of preparing such meat analogue composition by adding said mycelium ingredient to a composition comprising of at least one protein rich ingredient, at least one plant-based lipid rich ingredient, and optionally at least one compositional ingredient.
14. A method for producing a dairy analogue composition comprising the mycelium ingredient of any one of paragraphs 1 to 8 or 12 comprising the step of preparing such a dairy analogue composition by (1) forming a slurry comprising said mycelium ingredient with a composition comprising of at least one protein rich ingredient, at least one plant-based lipid rich ingredient, and at least one compositional ingredient, and (2) mixing the slurry with at least one compositional ingredient, specifically a texturizing agent or thickener or a carbohydrate-rich ingredient.
15. An edible product obtainable in the method of paragraph 13 or 14,
   preferably wherein the food product of paragraph 13 characterized in a soft texture, preferably has a hardness of 10-55N, springiness of 35-85%, cohesiveness of 15-70%, gumminess of 1-40N, a chewiness of 0.3-35N, a cutting strength of 1-25N and an adhesiveness of 0 N·s to −0.3 N·s, and/or
   preferably wherein the food product of paragraph 13 characterized in a non-soft or harder texture, preferably has a hardness of 30-100N, springiness of 20-70%, cohesiveness of 20-85%, gumminess of 6-85N, a chewiness of 1-60N, a cutting strength of 1-50N, and/or
   preferably wherein the food product of paragraph 14 characterized in a soft texture, preferably has a firmness of 1 to 20N, a spreadability of 30 to 100 N·s, a stickiness of-15 to-100N and a puncture force of 1 to 30 N·s, and/or
   preferably wherein the food product of paragraph 14 characterized in a non-soft or harder texture, preferably has a firmness of 20 to 100N, a spreadability of 1 to 20 N·s, a stickiness of −1 to-14N and a puncture force of 40 to 100 N·s.

It is to be understood that any of these embodiments can be combined with any other definition of mycelium ingredient A, B or C, as defined hereinabove, the methods of their production, or the respective food product.

1. An edible mycelium ingredient comprising non-differentiated mycelium biomass having an elemental composition of a C:N ratio of mycelium ranging between 2 and 12 and characterized by an equivalence umami concentration (EUC) of at least 500 monosodium glutamate g MSG/100 g,
wherein EUC is defined as:
$$E U C = \sum aibi + 1 \; 218 ( \sum aibi ) ( \sum ajbj ) ,$$
wherein
the EUC is expressed in g MSG/100 g,
ai is the concentration (g/100 g) of each umami amino acid Asp or Glu,
aj is the concentration (g/100 g) of each umami 5′-nucleotide 5′-IMP, 5′-GMP, or 5′-AMP, bi is the relative umami concentration (RUC) for each umami amino acid to MSG, defined as 1 for Glu and 0.077 for Asp and
bj is the RUC for each umami 5′-nucleotide defined as 1 for 5′-IMP, 2.3 for 5′-GMP and 0.18 for 5′-AMP.

2. (canceled)

3. The edible mycelium ingredient of claim 1, wherein the biomass has an elemental composition of a C:N ratio of mycelium ranging between 2 to 6 and/or an EUC of at least 2000 g MSG/100 g.

4-6. (canceled)

7. The edible mycelium ingredient of claim 1, wherein
(i) the inherent RNA level is 2 wt. % at most on a dry basis, without a further process step to reduce RNA;
(ii) the content of umami 5′ nucleotides (5′NMP) containing 5′-GMP and 5′-AMP is 20 g/kg at most;
(iii) the content of 5′-AMP and/or 5′-GMP is from 4.5 to 7 g/kg; and/or
(iv) the edible mycelium ingredient is substantially free of 5′ IMP.

8-12. (canceled)

13. The edible mycelium ingredient of claim 1, wherein
(i) the amount of branched-chain amino acids (BCAAs) is at least about 19 wt. % of total amount of amino acids present;
(ii) the amount of the BCAAs ranges between 50 and 150 mg/g and the amount of umami amino acids ranges between 70 and 100 mg/g;
(iii) the mycelia have an ergothioneine content ranging between 350 and 800 mg/kg;
(iv) the amount of the umami amino acids is at least about 19 wt. % of the total amount of amino acids present;
(v) at least 96 wt. % of polyunsaturated fatty acids comprises linoleic acid;
(vi) the mycelia have a DPPH radical-scavenging activity correlating with a Total Phenolic Content (TPC) ranging between 1 and 15 mg/ml;
(vii) the TPC, total flavanoid content (TFC) and polyphenol content range between 1 and 15 mg Gallic Acid Equivalents (GAE)/g, 1 and 15 mg Quercetin Equivalents (QE)/g and 100 and 1000 mg/kg, respectively, wherein the flavonoid content constitutes around 70 to 95% of the total phenolic content and wherein catechin and protocatechuic acid constitute each around 45 to 55% of the total polyphenols; and/or
(viii) the calorific value ranges between 300 and 600 Kcal/100 g with the ingredients having a thermal stability under N₂ atmosphere up to 190° C. at most.

14-21. (canceled)

22. The edible mycelium ingredient of claim 1, wherein the insoluble fiber content is between 20 and 60 wt. %.

23-24. (canceled)

25. The edible mycelium ingredient of claim 1, wherein
(i) the content of ergosterol is between 4 and 7 mg/g;
(ii) the carbohydrate content is 5 wt. % at most;
(iii) the content of uronic acids is between 0.1 and 5 wt. %
(iv) the chitin content ranges between 6 and 11 wt. %; and/or
(v) the beta-glucan content is at least 80% of the total glucans, wherein the total glucans content ranges between 10 and 20 wt. %.

26-29. (canceled)

30. The edible mycelium ingredient of claim 1, wherein, when freeze dried, for pore diameters below 1 mm, about 20% of pore-volume corresponds to a pore diameter between 1000 and 20 μm and about 80% of the pore-volume corresponds to a pore diameter between 20-2 μm having a most frequent pore diameter peak is equal to 5.5 μm.

31. The edible mycelium ingredient of claim 1, wherein the mycelium has a shear force of at least 10 N and water holding capacity ranging between 20-90% and a water release of 25 to 70%.

32-33. (canceled)

34. The edible mycelium ingredient of claim 33, wherein the mycelium is obtained from Pleurotus pulmonarius.

35. (canceled)

36. A method for producing the edible mycelium ingredient of claim 1 via submerged fermentation, comprising the step of culturing at least one fungal species in a fermentation medium, wherein the fermentation medium as provided at the beginning of the fermentation comprises of 5 to 60 g/L of carbon source, 0.1 to 60 g/L of nitrogen source, 0.01-15 g/L of minerals, and 0.01-50 mg/L of vitamins, wherein the medium comprises spent grain C5-sugar extract wherein the carbon to nitrogen ratio in this medium ranges between 10 and 25 and wherein at least 35 wt. % of extracted spent grains is characterized by particle size between 2 and 4 mm.

37. The method of claim 36, wherein the spent grain C5-sugar extract is sole carbon source and/or wherein the pH inside the fermenter is maintained between 4.0 and 5.0.

38. The method of claim 36 or 37, wherein:
(i) the carbon to nitrogen ratio in the fermentation medium ranges between 2 and 18;
(ii) the carbon to nitrogen ratio of the spent grain C5-sugar extract ranges between 5 and 18; and/or
(iii) the protein content of the spent grain C5-sugar extract ranges between 7 and 30 g/l protein.

39-46. (canceled)

47. The method of claim 36, further comprising the step of recovering the supernatant from the culture medium or part of it.

48-49. (canceled)

50. The method of claim 36, wherein upon separation of the biomass, the separated biomass is washed with water with a pH between 3 and 6.

51. (canceled)

52. A method for producing a soft or hard meat analogue composition comprising the edible mycelium ingredient of claim 1, comprising the step of preparing such meat analogue composition by adding said mycelium ingredient to a composition comprising of at least one protein rich ingredient, and at least one plant-based lipid rich ingredient.

53-54. (canceled)

55. A method for producing a dairy analogue composition comprising the edible mycelium ingredient of claim 1 comprising the step of preparing such a dairy analogue composition by (1) forming a slurry comprising said mycelium ingredient with a composition comprising of at least one protein rich ingredient, at least one plant-based lipid rich ingredient, and at least one compositional ingredient, and (2) mixing the slurry with at least one compositional ingredient.

56-58. (canceled)

59. An edible mycelium ingredient comprising non-differentiated mycelium biomass having an elemental composition of a C:N ratio of mycelium ranging between 8 and 12 and characterized by an EUC of less than 200 g MSG/100 g, wherein EUC is defined as:
$$E U C = \sum aibi + 1 \; 218 ( \sum aibi ) ( \sum ajbj ) ,$$
wherein the EUC is expressed in g MSG/100 g, ai is the concentration (g/100 g) of each umami amino acid Asp or Glu, aj is the concentration (g/100 g) of each umami 5′-nucleotide 5′-IMP, 5′-GMP, or 5′-AMP, bi is the relative umami concentration (RUC) for each umami amino acid to MSG, defined as 1 for Glu and 0.077 for Asp and bj is the RUC for each umami 5′-nucleotide defined as 1 for 5′-IMP, 2.3 for 5′-GMP and 0.18 for 5′-AMP.

60. (canceled)

61. The edible ingredient of claim 59, wherein the ingredient comprises Pleurotus pulmonarius.

62. The edible ingredient of claim 59, wherein
(i) the mycelia have an ergothioneine content of between 110 and 150 mg/kg;
(ii) the content umami 5′-nucleotides (5′NMP) containing 5′-guanosine monophosphate (GMP) and 5′-adenosine monophosphate (AMP) is up to 3 g/kg; and/or
(iii) the amount of BCAAs and the amount of umami amino acids range between 20 and 80 mg/g each respectively.

63-64. (canceled)

65. The edible ingredient of claim 59, having
(i) an insoluble fiber content between 40 and 50 wt. %;
(ii) a protein content between 30 and 50 wt. %, preferably between 30 and 40 wt. %; and/or
(iii) a total glucans content ranges between 25 and 50 wt. %.

66-67. (canceled)

68. A method for producing the edible mycelium ingredient of claim 59 characterized by EUC of less than 200 g MSG/100 g via submerged fermentation, comprising the step of culturing at least one fungal species in a fermentation medium, wherein the fermentation medium as provided at the beginning of the fermentation is characterized by a C:N ratio which ranges between 1 and 50, wherein the fermentation medium as provided at the beginning of the fermentation comprises 5 to 60 g/L of carbon source, 0.1 to 60 g/L of nitrogen source, 0.01-15 g/L of minerals, and 0.01-50 mg/L of vitamins and includes not more than 5 amino acids, including arginine and at most 4 further amino acids selected from Alanine, Cysteine, Glycine, Proline, Serine, Tyrosine, and Selenocysteine or selected from Phenylalanine, Valine, Tryptophan, Threonine, Isoleucine, Methionine, Histidine, Leucine, and Lysine.

69. The method of claim 68, wherein the medium includes arginine as the only amino acid.

70-72. (canceled)

73. An edible mycelium ingredient comprising mycelium biomass having an elemental composition of a C:N ratio of mycelium ranging between 6 and 8 and characterized by EUC of between 200 and 500 g MSG/100 g, wherein EUC is defined as:
$$E U C = \sum aibi + 1 \; 218 ( \sum aibi ) ( \sum ajbj ) ,$$
wherein the EUC is expressed in g MSG/100 g, ai is the concentration (g/100 g) of each umami amino acid Asp or Glu, aj is the concentration (g/100 g) of each umami 5′-nucleotide 5′-IMP, 5′-GMP, or 5′-AMP, bi is the relative umami concentration (RUC) for each umami amino acid to MSG, defined as 1 for Glu and 0.077 for Asp and bj is the RUC for each umami 5′-nucleotide defined as 1 for 5′-IMP, 2.3 for 5′-GMP and 0.18 for 5′-AMP.

74. The edible mycelium ingredient of claim 73, wherein:
(i) the inherent RNA level is below 1.88 wt. % on a dry basis, without a further process step to reduce RNA;
(ii) the mycelia have an ergothioneine content ranges between 70 and 100 mg/kg;
(iii) the content of umami 5′ nucleotides (5′NMP) containing 5′-GMP and 5′-AMP is up to 6 g/kg;
(iii) the amount of BCAAs and the amount of umami amino acids range between 40 and 100 mg/g each, respectively;
(iv) the protein content is between 30 and 40 wt. %; and/or
(v) the total glucan content ranges between 20 and 35 wt. %.

75-77. (canceled)

78. The edible mycelium ingredient of claim 73, wherein the insoluble fiber content is between 30 and 60 wt. %.

79-80. (canceled)

81. A method for producing the edible mycelium ingredient of claim 73 via submerged fermentation, comprising the step of culturing at least one fungal species in a fermentation medium, wherein the fermentation medium as provided at the beginning of the fermentation comprises of 5 to 60 g/L of carbon source, 0.1 to 60 g/L of nitrogen source, 0.01-15 g/L of minerals, and 0.01-50 mg/L vitamins wherein the carbon to nitrogen ratio in this medium ranges between 10 and 25, wherein the pH inside the fermenter is maintained between 4.0 and 5.0, and wherein upon separation of the biomass, the separated biomass is washed with water with a pH between 3 and 7.

82. The method of claim 81, wherein the carbon to nitrogen ratio in the this-medium ranges between 16 and 18.

83. An edible product, comprising from 1 to 99 wt. % of the editable mycelium ingredient of claim 73.

84. The edible product of claim 83, wherein said mycelium ingredient is characterized by an EUC of about 300 g MSG/100 g and/or an insoluble fiber content of between 30 and 40 wt. %.

85. (canceled)

86. An edible product, comprising from 1 to 99 wt. % of the editable mycelium ingredient of claim 59.

87. The edible product of claim 86, wherein said mycelium ingredient is characterized by an EUC of less than 50 g MSG/100 g and/or an insoluble fiber content of between 40 and 60 wt.

88-92. (canceled)

93. The edible mycelium ingredient of claim 30, comprising Pleurotus pulmonarius mycelium biomass having an elemental composition of a C:N ratio of mycelium ranging between 2 and 12 and characterized by an equivalence umami concentration (EUC) of at least 30 monosodium glutamate g MSG/100 g.

94. The edible mycelium ingredient of claim 93, wherein:
(i) the insoluble fiber content is between 20 and 60 wt. %;
(ii) the protein content is between 10 and 65 wt. %; and/or
(iii) the inherent RNA level is 2 wt. % at most on a dry basis, without a further process step to reduce RNA.

95-96. (canceled)

97. A method for producing the edible mycelium ingredient of claim 93 via submerged fermentation, comprising the step of culturing at least one fungal strain in a fermentation medium, wherein the fermentation medium as provided at the beginning of the fermentation is characterized by C:N ratio which ranges between 5 and 50, wherein the fermentation medium as provided at the beginning of the fermentation comprises of 5 to 60 g/L of carbon source, 0.1 to 60 g/L of nitrogen source, 0.01-15 g/L of minerals, and 0.01-50 mg/L of vitamins, characterized in that the medium includes arginine and glutamate as the only amino acids.

98. (canceled)

99. An edible product comprising the edible mycelium ingredient of claim 93 ranging from 1 to 99 wt. %.

100-101. (canceled)
